(19) United States
(12) Patent Application Publication
(10) Pub. No.: US 2010/0069360 A1
(43) Pub. Date: Mar. 18, 2010

(54) ORGANIC COMPOUNDS

(76) Inventors: Laszlo Revesz, Therwil (CH); Achim Schlapbach, Lorrach (DE); Rudolf Walchli, Basel (CH)

Correspondence Address: Fanelli Strain & Haag PLLC, 1455 Pennsylvania Ave., N.W., suite 400, Washington, DC, 20004, US

(21) Appl. No.: 12/439,603

(22) PCT Filed: Aug. 28, 2007
(86) PCT No.: PCT/EP07/07510

§ 371 Date: Mar. 2, 2009

(30) Foreign Application Priority Data
Aug. 30, 2006 (EP) 06119817.2

(51) Int. Cl. A61K 31/437; C07D 471/04; C07D 471/14; C07D 498/22; A61K 31/5365; A61K 31/55; A61K 31/5025; A61K 31/5377; A61K 31/496; A61P 37/00; A61K 31/52

(52) U.S. Cl. 514/212.06; 546/84; 546/64; 544/63; 546/70; 540/521; 544/233; 544/125; 544/361; 514/292; 514/229.5; 514/285; 514/248; 514/232.8; 514/253.02; 514/263.2

(57) ABSTRACT

A compound of formula (I) or a pharmaceutically acceptable salt or prodrug ester thereof:

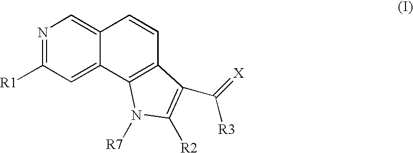

wherein the groups R1, R2, R3, R7 and X are as defined in the specification.

The present invention relates to novel aromatic compounds as inhibitors of mitogen-activated protein kinase-activated protein kinase-2 (MK2 or MAPKAP kinase-2).

Accordingly the present invention provides a compound of formula (I) or a pharmaceutically acceptable salt or a pharmaceutically-acceptable and -cleavable ester, or acid addition salt thereof:

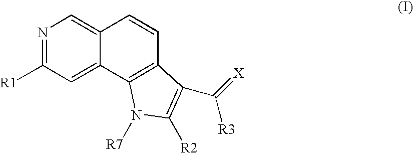

wherein

R1 is selected from: halo, cyano, hydroxyl, mercapto, optionally substituted (aryl, aryl-C₁-C₆ alkyl, aryl-C₂-C₆ alkenyl, monocyclic heteroaryl, heteroaryl-C₁-C₆ alkyl, heteroaryl-C₂-C₆ alkenyl, arylamino, heteroarylamino, aryloxy, heteroaryloxy, C₁-C₆ alkyl, C₃-C₇ cycloalkyl, C₁-C₆ alkoxy, C₁-C₆ alkylamino, C₂-C₆ alkenyl, C₂-C₆ alkynyl, heterocycloalkyl, heterocycloalkyl-C₁-C₆ alkyl, heterocycloalkyl-C₂-C₆ alkenyl, heterocycloalkylamino, heterocycloalkyloxy, amino),

wherein the optional substituents on R1 are selected from halo, cyano, hydroxyl, mercapto, sulfonyl, amino, C₁-C₆ alkylamino, di-C₁-C₆ alkylamino, aryl, monocyclic heteroaryl, C₁-C₆ alkyl, C₁-C₆ alkoxy, C₃-C₇ cycloalkyl, C₃-C₇ heterocycloalkyl, carboxyl, carbonyl C₁-C₇ alkyl, each of which, where applicable, may be optionally substituted by C₁-C₆ alkyl, C₃-C₇ cycloalkyl, C₃-C₇ cycloalkyl, C₃-C₇ heterocycloalkyl, C₁-C₆ alkoxy, C₁-C₆ alkenyl, C₁-C₆ alkynyl, halo, hydroxyl, mercapto, cyano, amino, C3-C7 heterocycloalkyl carbonyl; each of which, where applicable, may be optionally substituted by C₁-C₆ alkyl, C₃-C₇ cycloalkyl, C₃-C₇ cycloalkyl, C₃-C₇ heterocycloalkyl, C₁-C₆ alkoxy, C₁-C₆ alkenyl, C₁-C₆ alkynyl, halo, hydroxyl, mercapto, cyano, amino, C3-C7 heterocycloalkyl carbonyl;

X is O, S or NOH;

R2 represents the group —C(A)(Q)-Y

wherein Q is H or C₁-C₆ alkyl;

A is H or C₁-C₆ alkyl;

Y is amino, aminooxy, hydroxyl, C₁-C₆ alkoxy, C₁-C₆ alkylamino or hydrazine which in each case may be optionally substituted,

the optional substituents on Y being selected from C₁-C₆ alkyl, halo, hydroxyl;

R3 is —OH, —OR4 or —NHR4,

wherein R4 is H or C₁-C₆ alkyl;

or R2 and R3 are joined to denote collectively the group —R2-R3- to form a 5-, 6 or 7-membered ring, the collective group —R2-R3- being selected from:

—(CH₂)ₙNR5-, —CH₂ONH—, —(CH₂)—, —CH═N—NH—, —(CH₂)ₙ—NR6-NH—

wherein R5 is selected from H or optionally substituted (C₁-C₆ alkyl, aryl-C₁-C₆ alkyl, heteroaryl-C₁-C₆ alkyl, C₃-C₇ cylcloakyl-C₁-C₆ alkyl, C₃-C₇ heterocyldoalkyl-C₁-C₆ alkyl); the optional substituents on R5 being one or more groups independently selected from halo, C₁-C₆ alkyl, C₁-C₆ alkoxy, amino, trifluoromethyl, sulfonyl, hydroxyl;

and R6 is selected from H or optionally substituted C₁-C₆ alkyl, carbonyl, sulfonyl; the optional substituents on R6 being one or more groups independently selected from C₁-C₆ alkyl, lower alkoxy, amino, alkylamino, hydroxyl;

wherein n is 1, 2 or 3;

and R7 is selected from H and optionally substituted C₁-C₆ alkyl, the optional substituents being selected from amino, hydroxyl, halo and carboxy.

According to the invention in a second aspect there is provided a compound of formula (II) or a pharmaceutically acceptable salt or a pharmaceutically-acceptable and -cleavable ester, or acid addition salt thereof:

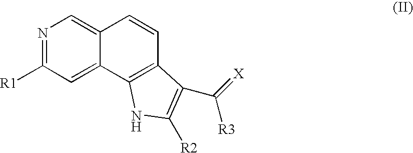

wherein

R1 is selected from: H, halo, cyano, hydroxyl, mercapto, optionally substituted (aryl, aryl-C₁-C₆ alkyl, aryl-C₂-C₆ alkenyl, monocyclic heteroaryl, heteroaryl-C₁-C₆ alkyl, heteroaryl-C₂-C₆ alkenyl, arylamino, heteroarylamino, aryloxy, heteroaryloxy, C₁-C₆ alkyl, C₃-C₇ cycloalkyl, C₁-C₆ alkoxy, C₁-C₆ alkylamino, C₂-C₆ alkenyl, C₂-C₆ alkynyl, heterocycloalkyl, heterocycloalkyl-C₁-C₆ alkyl, heterocycloalkyl-C₂-C₆ alkenyl, heterocycloalkylamino, heterocycloalkyloxy, amino),

wherein the optional substituents on R1 are selected from halo, cyano, hydroxyl, mercapto, sulfonyl, amino, aryl, monocyclic heteroaryl, C₁-C₆ alkyl, C₁-C₆ alkoxy, C₃-C₇ cycloalkyl, C₃-C₇ heterocycloalkyl, carboxyl, each of which, where applicable, may be optionally substituted by C₁-C₆ alkyl, C₃-C₇ cycloalkyl, C₃-C₇ cycloalkyl, C₃-C₇ heterocycloalkyl, C₁-C₆ alkoxy, C₁-C₆ alkenyl, C₁-C₆ alkynyl, halo, hydroxyl, mercapto, cyano, amino;

X is O, S or NOH;

R2 represents the group —C(A)(Q)-Y

wherein Q is H or C₁-C₆ alkyl;

A is H or C₁-C₆ alkyl;

Y is amino, aminooxy, hydroxyl, C₁-C₆ alkoxy, C₁-C₆ alkylamino or hydrazine which in each case may be optionally substituted,

the optional substituents on Y being selected from C₁-C₆ alkyl, halo, hydroxyl;

R3 is —OH, —OR4 or —NHR4,

wherein R4 is H or C₁-C₆ alkyl;

or R2 and R3 are joined to denote collectively the group —R2-R3- to form a 5-, 6 or 7-membered ring, the collective group —R2-R3- being selected from:

—(CH₂)ₙNR5-, —CH₂ONH—, —(CH₂)—, —CH═N—NH—, —(CH₂)ₙ—NH—NH—

wherein R5 is selected from H or optionally substituted (C₁-C₆ alkyl, aryl-C₁-C₆ alkyl, heteroaryl-C₁-C₆ alkyl, C₃-C₇ cylcloakyl-C₁-C₆ alkyl, C₃-C₇ heterocylcloalkyl-C₁-C₆ alkyl); the optional substituents on R5 being one or more groups independently selected from halo, C₁-C₆ alkyl, C₁-C₆ alkoxy, amino, trifluoromethyl, sulfonyl, hydroxyl;

and wherein n is 1, 2 or 3.

In a preferred embodiment of the invention with respect to formula (I) and (II), R1 is halo or optionally substituted (aryl, monocyclic heteroaryl, aryl-C₂-C₆ alkenyl, aryloxy, C1-C₆ alkylamino), the optional substituents on R1 being as previously defined.

When R1 contains a heteroaryl group, preferably it is a monocyclic heteroaryl group.

Preferably, the optional substituents on R1 are one or more groups independently selected from halo, C₁-C₆ alkyl, C₁-C₆ alkoxy, sulfonyl, heteroaryl, each of which, where possible, may be optionally substituted by C₁-C₆ alkyl, C₃-C₇ cycloalkyl, C₁-C₆ alkoxy, C₁-C₆ alkenyl, C₁-C₆ alkynyl, halo, hydroxyl, sulfonyl, amino, aryl, heteroaryl, C₃-C₆ heterocycloalkyl.

In a more preferred embodiment, the optional substituents on R1 are one or more groups independently selected from halo, C₁-C₆ alkoxy, sulfonyl, trifluoromethyl, C₃-C₆ heterocycloalkyl.

In a preferred embodiment, R1 is aryl-C₂-C₆ alkenyl, more preferably aryl-ethylenyl, yet more preferably styryl. An alternative preferred group for R1 is phenyl-C₂-C₆ alkenyl, more preferably styryl, the optional substituents on R1 being as defined previously.

Preferably, R1 is halo, optionally substituted (phenyl, pyridyl or styryl), the optional substituents where applicable on R1 being as defined previously.

Alternatively preferably, R1 is optionally substituted (aryl or aryl-C₁-C₆ alkenyl), the optional substituents where applicable on R1 being as defined previously.

Yet more preferably, R1 is a phenyl, pyridyl or styryl group each of which may be optionally substituted as indicated previously.

X is preferably O or NOH; more preferably X is O.

In a preferred embodiment of the invention, R2 represents the group —CH(Q)-Y wherein Q is H; and Y is selected from amino, aminooxy, C₁-C₆ alkylamino, hydrazine which in each case may be optionally substituted, the optional substituents on Y being selected from C₁-C₆ alkyl, halo, hydroxyl.

More preferably, R2 represents the group —CH(Q)-Y wherein Q is H; and Y is selected from amino, methylamino, aminooxy, methoxy and hydrazino.

In a preferred embodiment of the invention, R3 is OH. Alternatively preferably, R3 is NH₂.

In an alternative preferred embodiment, R2 and R3 are joined to denote collectively the group —R2-R3- to form a 5-, 6 or 7-membered ring, the collective group —R2-R3- being selected from:

—(CH₂)ₙNR5-, —CH₂ONH—, —CH═N—NH—, —(CH₂)ₙ—NR6-NH—

wherein R5 is selected from H or optionally substituted (C₁-C₆ alkyl, aryl-C₁-C₆ alkyl, heteroaryl-C₁-C₆ alkyl, C₃-C₇ cylcloakyl-C₁-C₆ alkyl, C₃-C₇ heterocylcloalkyl-C₁-C₆ alkyl); the optional substituents on R5 being one or more groups independently selected from halo, C₁-C₆ alkyl, C₁-C₆ alkoxy, amino, trifluoromethyl, sulfonyl, hydroxyl.

and R6 is selected from H or optionally substituted C₁-C₆ alkyl, carbonyl, sulfonyl; the optional substituents on R6 being one or more groups independently selected from C₁-C₆ alkyl, lower alkoxy, amino, alkylamino, hydroxyl.

and wherein n is 1, 2 or 3.

More preferably, R5 is H or optionally substituted (aryl-C₁-C₆ alkyl or heteroaryl-C₁-C₆ alkyl), the substituents being as listed above.

More preferably, R5 is H or optionally substituted (aryl-methyl or heteroaryl-methyl), the substituents being as listed above.

More preferably, R5 is H or optionally substituted (benzyl or pyridylmethyl), the substituents being as listed above.

R7 is preferably H or methyl, yet more preferably H.

For the avoidance of doubt, the terms listed below are to be understood to have the following meaning throughout the present description and claims:

The term “lower”, when referring to organic radicals or compounds means a compound or radical with may be branched or unbranched with up to and including 7 carbon atoms.

An alkyl group may be branched, unbranched or cyclic. C₁-C₆ alkyl represents, for example: methyl, ethyl, propyl, butyl, isopropyl, isobutyl, tertiary butyl or 2,2-dimethylpropyl.

An alkoxy group may be branched or unbranched. C₁-C₆ alkoxy represents, for example: methoxy, ethoxy, propoxy, butoxy, isopropoxy, isobutoxy or tertiary butoxy. Alkoxy includes cycloalkyloxy and cycloalkyl-alkyloxy.

An alkene, alkenyl or alkenoxy group is branched or unbranched and contains 2 to 7 carbon atoms, preferably 2 to 4 carbon atoms and contains at least one carbon-carbon double bond. Alkene, alkenyl or alkenoxy represents for example vinyl, prop-1-enyl, allyl, butenyl, isopropenyl or isobutenyl and the oxy equivalents thereof.

An akyne or alkynyl group is branched or unbranched and contains 2 to 7 carbon atoms, preferably 1 to 4 carbon atoms and contains at least one carbon-carbon triple bond. Lower alkyne or lower alkynyl or lower alkenyloxy represents for example ethynyl or propynyl.

In the present application, oxygen containing substituents, e.g. alkoxy, alkenyloxy, alkynyloxy, carbonyl, etc. encompass their sulphur containing homologues, e.g. thioalkyl, alkyl-thioalkyl, thioalkenyl, alkenyl-thioalkyl, thioalkynyl, thiocarbonyl, sulphone, sulphoxide etc.

Halo or halogen represents chloro, fluoro, bromo or iodo.

Aryl represents carbocyclic aryl or biaryl.

Carbocyclic aryl is an aromatic cyclic hydrocarbon containing from 6 to 18 ring atoms. It can be monocyclic, bicyclic or tricyclic, for example naphthyl, phenyl, or phenyl mono-, di- or trisubstituted by one, two or three substituents.

Heterocyclic aryl or heteroaryl is an aromatic monocyclic or bicyclic hydrocarbon containing from 5 to 18 ring atoms one or more of which are heteroatoms selected from O, N or S. Preferably there are one to three heteroatoms. Heterocyclic aryl represents, for example: pyridyl, indolyl, quinoxalinyl, quinolinyl, isoquinolinyl, benzothienyl, benzofuranyl, benzthiophenyl, benzopyranyl, benzothiopyranyl, furanyl, pyrrolyl, thiazolyl, oxazolyl, isoxazolyl, triazolyl, tetrazolyl, pyrazolyl, imidazolyl, thienyl, oxadiazolyl, benzimidazolyl, benzthiazolyl, benzoxazolyl, Heterocyclic aryl also includes such substituted radicals.

Cycloalkyl represents a cyclic hydrocarbon containing from 3 to 12 ring atoms preferably from 3 to 6 ring atoms. Cydoalkyl represents, for example: cyclopropyl, cyclobutyl, cyclopentyl, or cyclohexyl. The cycloalkyl may optionally be substituted.

Heterocycloalkyl represents a mono-, di- or tricyclic hydrocarbon which may be saturated or unsaturated and which contains one or more, preferably one to three heteroatoms selected from O, N or S. Preferably it contains between three and 18 ring atoms, more preferably between 3 and 8 ring atoms. Heterocycloalkyl represents for example morpholinyl, piperazinyl, piperidinyl, imidazolidinyl, pyrrolidinyl, pyrazolidinyl. The term heterocycloalkyl is intended also to include bridged heterocycloalkyl groups such as 3-hydroxy-8-aza-bicyclo[3.2.1]oct-8-yl or 2,6-diaza-tricyclo[3.3.1.1*3,7]dec-1-yl.

Pharmaceutically acceptable salts include acid addition salts with conventional acids, for example mineral acids, e.g. hydrochloric acid, sulfuric or phosphoric acid, or organic acids, for example aliphatic or aromatic carboxylic or sulfonic acids, e.g. acetic, trifluoroacetic, propionic, succinic, glycolic, lactic, malic, tartaric, citric, ascorbic, maleic, fumaric, hydroxylmaleic, pyruvic, pamoic, methanesulfonic, toluenesulfonic, naphthalenesulfonic, sulfanilic or cyclohexylsulfamic acid; also amino acids, such as arginine and lysine. For compounds of the invention having acidic groups, for example a free carboxy group, pharmaceutically acceptable salts also represent metal or ammonium salts, such as alkali metal or alkaline earth metal salts, e.g. sodium, potassium, magnesium or calcium salts, as well as ammonium salts, which are formed with ammonia or suitable organic amines.

The agents of the invention which comprise free hydroxyl groups may also exist in the form of pharmaceutically acceptable, physiologically cleavable esters, and as such are included within the scope of the invention. Such pharmaceutically acceptable esters are preferably prodrug ester derivatives, such being convertible by solvolysis or cleavage under physiological conditions to the corresponding agents of the invention which comprise free hydroxyl groups. Suitable pharmaceutically acceptable prodrug esters are those derived from a carboxylic acid, a carbonic acid monoester or a carbamic acid, advantageously esters derived from an optionally substituted lower alkanoic acid or an arylcarboxylic acid.

Preferred compounds of formula (I) are:

2-Aminomethyl-8-((E)-styryl)-1H-pyrrolo[2,3-f]isoquinoline-3-carboxylic acid hydrochloride

2-((E)-Styryl)-9,10-dihydro-8H-3,8,10-triaza-pentaleno[2,1-a]naphthalen-7-one

2-Aminomethyl-8-chloro-1H-pyrrolo[2,3-f]isoquinoline-3-carboxylic acid hydrochloride

2-Chloro-9,10-dihydro-8H-3,8,10-triaza-pentaleno[2,1-a]naphthalen-7-one

2-Aminooxymethyl-8-((E)-styryl)-1H-pyrrolo[2,3-f]isoquinoline-3-carboxylic acid hydrochloride

2-((E)-Styryl)-10,11-dihydro-9-oxa-3,8,11-triaza-benzo[a]fluoren-7-one

2-Chloro-8,9,10,11-tetrahydro-pyrido[4,3-a]carbazol-7-one

2-Chloro-8,9,10,11-tetrahydro-pyrido[4,3-a]carbazol-7-one oxime

2-Chloro-9,10,11,12-tetrahydro-8H-3,8,12-triaza-naphtho[2,1-a]azulen-7-one

2-((E)-Styryl)-9,10,11,12-tetrahydro-8H-3,8,12-triaza-naphtho[2,1-a]azulen-7-one

2-(4-Fluoro-phenyl)-9,10,11,12-tetrahydro-8H-3,8,12-triaza-naphtho[2,1-a]azulen-7-one

2-Aminooxymethyl-8-chloro-1H-pyrrolo[2,3-f]isoquinoline-3-carboxylic acid hydrochloride

2-Chloro-10,11-dihydro-9-oxa-3,8,11-triaza-benzo[a]fluoren-7-one

8-Chloro-2-hydrazinomethyl-1H-pyrrolo[2,3-f]isoquinoline-3-carboxylic acid hydrochloride

2-Chloro-8,11-dihydro-3,8,9,11-tetraaza-benzo[a]fluoren-7-one

2-Chloro-8,9,10,11-tetrahydro-3,8,9,11-tetraaza-benzo[a]fluoren-7-one

2-(4-Methoxy-phenyl)-9-methyl-8,9,10,11-tetrahydro-3,8,9,11-tetraaza-benzo[a]fluoren-7-one

2-(4-Methoxyphenyl)-9,10,11,12-tetrahydro-8H-3,8,12-triaza-naphtho[2,1-a]azulen-7-one

2-Methoxymethyl-8-((E)-styryl)-1H-pyrrolo[2,3-f]isoquinoline-3-carboxylic acid amide

2-Methylaminomethyl-8-((E)-styryl)-1H-pyrrolo[2,3-f]isoquinoline-3-carboxylic acid hydrochloride

8-Methyl-2-((E)-styryl)-9,10-dihydro-8H-3,8,10-triaza-pentaleno[2,1-a]naphthalen-7-one

2-(4-Hydroxy-phenyl)-9,10-dihydro-8H-3,8,10-triaza-pentaleno[2,1-a]naphthalen-7-one

2-[(E)-2-(4-Methoxy-phenyl)-vinyl]-9,10-dihydro-8H-3,8,10-triaza-pentaleno[2,1-a]naphthalen-7-one

2-[(E)-2-(4-Morpholin-4-ylmethyl-phenyl)-vinyl]-9,10-dihydro-8H-3,8,10-triaza-pentaleno[2,1-a]naphthalen-7-one

2-{(E)-2-[3-(2-Morpholin-4-yl-ethyl)-phenyl]-vinyl}-9,10-dihydro-8H-3,8,10-triaza-pentaleno[2,1-a]naphthalen-7-one

8-Benzyl-2-((E)-styryl)-9,10-dihydro-8H-3,8,10-triaza-pentaleno[2,1-a]naphthalen-7-one

2-(3-Methoxy-phenyl)-9,10-dihydro-8H-3,8,10-triaza-pentaleno[2,1-a]naphthalen-7-one

2-[3-(3-Methoxy-propoxy)-phenyl]-9,10-dihydro-8H-3,8,10-triaza-pentale

no[2,1-a]naphthalen-7-one

2-Pyridin-3-yl-9,10-dihydro-8H-3,8,10-triaza-pentaleno[2,1-a]naphthalen-7-one

2-(4-Methoxy-phenyl)-9,10-dihydro-8H-3,8,10-triaza-pentaleno[2,1-a]naphthalen-7-one

2-(2-Fluoro-phenyl)-9,10-dihydro-8H-3,8,10-triaza-pentaleno[2,1-a]naphthalen-7-one

2-(3-Methanesulfonyl-phenyl)-9,10-dihydro-8H-3,8,10-triaza-pentaleno[2,1-a]naphthalen-7-one

2-(2-Trifluoromethyl-phenyl)-9,10-dihydro-8H-3,8,10-triaza-pentaleno[2,1-a]naphthalen-7-one

2-(3-Fluoro-phenylamino)-9,10-dihydro-8H-3,8,10-triaza-pentaleno[2,1-a]naphthalen-7-one

2-[(E)-2-(4-Morpholin-4-ylmethyl-phenyl)-vinyl]-9,10,11,12-tetrahydro-8H-3,8,12-triaza-naphtho[2,1-a]azulen-7-one

2-Pyridin-3-yl-9,10,11,12-tetrahydro-8H-3,8,12-triaza-naphtho[2,1-a]azulen-7-one

2-Chloro-8,9,10,11-tetrahydro-3,8,11-triaza-benzo[a]fluoren-7-one

2-{(E)-2-[4-(2-Hydroxy-2-methyl-propoxy)-phenyl]-vinyl}-8,9,10,11-tetrahydro-3,8,11-triaza-benzo[a]fluoren-7-one

2-[(E)-2-(3-Morpholin-4-yl-phenyl)-vinyl]-8,9,10,11-tetrahydro-3,8,11-triaza-benzo[a]fluoren-7-one

2-[(E)-2-(3-Morpholin-4-yl-phenyl)-vinyl]-9,10-dihydro-8H-3,8,10-triaza-pentaleno[2,1-a]naphthalen-7-one

2-[(E)-2-(3-Morpholin-4-yl-phenyl)-vinyl]-9,10,11,12-tetrahydro-8H-3,8,12-triaza-naphtho[2,1-a]azulen-7-one

2-[(E)-2-(3-Morpholin-4-ylmethyl-phenyl)-vinyl]-9,10-dihydro-8H-3,8,10-triaza-pentaleno[2,1-a]naphthalen-7-one

2-[(E)-2-(3-Morpholin-4-ylmethyl-phenyl)-vinyl]-8,9,10,11-tetrahydro-3,8,11-triaza-benzo[a]fluoren-7-one

2-[(E)-2-(3-Morpholin-4-ylmethyl-phenyl)-vinyl]-9,10,11,12-tetrahydro-8H-3,8,12-triaza-naphtho[2,1-a]azulen-7-one

2-{(E)-2-[3-(2-Morpholin-4-yl-ethyl)-phenyl]vinyl}-8,9,10,11-tetrahydro-3,8,11-triaza-benzo[a]fluoren-7-one

2-{(E)-2-[3-(2-Morpholin-4-yl-ethyl)-phenyl]-vinyl}-9,10,11,12-tetrahydro-8H-3,8,12-triaza-naphtho[2,1-a]azulen-7-one

2-{(E)-2-[3-(2-Morpholin-4-yl-2-oxo-ethyl]-phenyl}-vinyl)-9,10-dihydro-8H-3,8,10-triaza-pentaleno[2,1-a]naphthalen-7-one

2-{(E)-2-[3-(2-Morpholin-4-yl-2-oxo-ethyl)-phenyl]vinyl}-8,9,10,11-tetrahydro-3,8,11-triaza-benzo[a]fluoren-7-one

2-{(E)-2-[3-(2-Morpholin-4-yl-2-oxo-ethyl)-phenyl]vinyl}-9,10,11,12-tetrahydro-8H-3,8,12-triaza-naphtho[2,1-a]azulen-7-one

2-((E)-2-{3-[2-(4-Methyl-piperazin-1-yl)-2-oxo-ethyl]-phenyl}-vinyl)-9,10-dihydro-8H-3,8,10-triaza-pentaleno[2,1-a]naphthalen-7-one

2-((E)-2-{3-[2-(4-Methyl-piperazin-1-yl)-2-oxo-ethyl]-phenyl}-vinyl)-8,9,10,11-tetrahydro-3,8,11-triaza-benzo[a]fluoren-7-one

2-((E)-2-{3-[2-(4-Methyl-piperazin-1-yl)-2-oxo-ethyl]-phenyl}-vinyl)-9,10,11,12-tetrahydro-8H-3,8,12-triaza-naphtho[2,1-a]azulen-7-one

11-Methyl-2-[(E)-2-(3-morpholin-4-yl-phenyl)-vinyl]-8,9,10,11-tetrahydro-3,8,11-triaza-benzo[a]fluoren-7-one

11-Methyl-2-{(E)-2-[3-(2-morpholin-4-yl-ethyl)-phenyl]vinyl}-8,9,10,11-tetrahydro-3,8,11-triaza-benzo[a]fluoren-7-one

1,1-Dimethyl-4-(2-{3-[(E)-2-(10-methyl-7-oxo-7,8,9,10-tetrahydro-3,8,10-triaza-pentaleno[2,1-a]naphthalen-2-yl)-vinyl]phenyl}-acetyl)piperazin-1-ium iodide

2-[(E)-2-(4-Morpholin-4-ylmethyl-phenyl)-vinyl]-8,9,10,11-tetrahydro-3,8,11-triaza-benzo[a]fluoren-7-one

2-[3-(2-Morpholin-4-yl-ethoxy)-phenyl]-9,10-dihydro-8H-3,8,10-triaza-pentaleno[2,1-a]naphthalen-7-one

2-[3-(2-Morpholin-4-yl-ethoxy)-phenyl]-8,9,10,11-tetrahydro-3,8,11-triaza-benzo[a]fluoren-7-one

2-[3-(2-Morpholin-4-yl-ethoxy)-phenyl]-9,10,11,12-tetrahydro-8H-3,8,12-triaza-naphtho[2,1-a]azulen-7-one

2-{4-[2-(4-Methyl-piperazin-1-yl)-2-oxo-ethyl]-phenyl}-8,9,10,11-tetrahydro-3,8,11-triaza-benzo[a]fluoren-7-one

2-{3-[2-(4-Methyl-piperazin-1-yl)-2-oxo-ethyl]-phenyl}-8,9,10,11-tetrahydro-3,8,11-triaza-benzo[a]fluoren-7-one

2-{3-[2-(4-Methyl-piperazin-1-yl)-2-oxo-ethyl]-phenyl}-9,10,11,12-tetrahydro-8H-3,8,12-triaza-naphtho[2,1-a]azulen-7-one

2-[5-(2-Morpholin-4-yl-ethoxy)-pyridin-3-yl]-9,10-dihydro-8H-3,8,10-triaza-pentaleno[2,1-a]naphthalen-7-one

2-[5-(2-Morpholin-4-yl-ethoxy)-pyridin-3-yl]-8,9,10,11-tetrahydro-3,8,11-triaza-benzo[a]fluoren-7-one

2-[5-(2-Morpholin-4-yl-ethoxy)-pyridin-3-yl]-9,10,11,12-tetrahydro-8H-3,8,12-triaza-naphtho[2,1-a]azulen-7-one

2-[5-(2-Methoxy-ethoxy)-pyridin-3-yl]-9,10,11,12-tetrahydro-8H-3,8,12-triaza-naphtho[2,1-a]azulen-7-one

2-[5-(2-Methoxy-ethoxy)-pyridin-3-yl]-9,10-dihydro-8H-3,8,10-triaza-pentaleno[2,1-a]naphthalen-7-one

2-[5-(2-Methoxy-ethoxy)-pyridin-3-yl]-8,9,10,11-tetrahydro-3,8,11-triaza-benzo[a]fluoren-7-one

2-Pyridin-3-yl-9,10,11,12-tetrahydro-8H-3,8,12-triaza-naphtho[2,1-a]azulen-7-one

2-(5-Methoxy-pyridin-3-yl)-9,10-dihydro-8H-3,8,10-triaza-pentaleno[2,1-a]naphthalen-7-one

2-(6-Methoxy-pyridin-3-yl)-9,10-dihydro-8H-3,8,10-triaza-pentaleno[2,1-a]naphthalen-7-one

2-(6-Dimethylamino-pyridin-3-yl)-9,10-dihydro-8H-3,8,10-triaza-pentaleno[2,1-a]naphthalen-7-one

2-{(E)-2-[4-(2-Hydroxy-2-methyl-propoxy)-phenyl]-vinyl}-9,10,11,12-tetrahydro-8H-3,8,12-triaza-naphtho[2,1-a]azulen-7-one

2-(3-Fluoro-4-methoxy-phenyl)-8,9,10,11-tetrahydro-3,8,11-triaza-benzo[a]fluoren-7-one

2-(3-Chloro-4-propoxy-phenyl)-8,9,10,11-tetrahydro-3,8,11-triaza-benzo[a]fluoren-7-one

2-(3-Fluoro-4-methoxy-phenyl)-9,10-dihydro-8H-cyclopenta[4,5]pyrrolo[2,3-f]isoquinolin-7-one

2-(3-Chloro-4-propoxy-phenyl)-9,10-dihydro-8H-cyclopenta[4,5]pyrrolo[2,3-f]isoquinolin-7-one.

The invention in a second aspect provides a compound of formula (I) or (II) or a pharmaceutically-acceptable and—cleavable ester, or acid addition salt thereof for use as a pharmaceutical.

The invention in a third aspect provides the use of a compound of formula (I) or (II) or a pharmaceutically-acceptable and -cleavable ester, or acid addition salt thereof in the manufacture of a medicament for the treatment of an autoimmune disease or condition.

The invention in a fourth aspect provides the use of a compound of formula (I) or (II) or a pharmaceutically-acceptable and -cleavable ester, or acid addition salt thereof for the treatment of cytokine mediated, e.g. TNF alpha mediated and/or MK2 related conditions.

The invention in a fifth aspect provides a method of treatment of cytokine mediated, e.g. TNF alpha mediated and/or MK2 related conditions comprising administering an effective amount of a compound of formula (I) or a pharmaceutically-acceptable and -cleavable ester, or acid addition salt thereof to a patient in need of such treatment.

The invention in a sixth aspect provides a pharmaceutical composition comprising a compound of formula (I) or (II) or a pharmaceutically-acceptable and -cleavable ester, or acid addition salt thereof in association with a pharmaceutically acceptable excipient, diluent or carrier.

In a seventh aspect the invention provides a process for preparing a compound of formula (I) or (II) in free or salt form, comprising the step of:

(a) For compounds of formula (I) or (II) wherein R1 is directly bonded via a C atom, by a Suzuki or Stille coupling of a compound of formula (V)

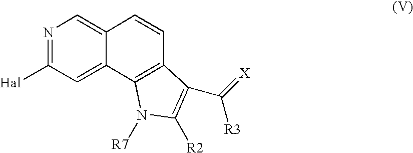

wherein Hal is a halogen, e.g. Cl, and R2, R3 and X are as defined with respect to the corresponding formula (I),

with a compound of formula R1-B wherein B represents the appropriate group for a Suzuki or Stille coupling reagent, e.g. boronic acid or ester, or 3-(1,1,1-tributylstannyl)- respectively, under suitable reaction conditions;

(b) For compounds of formula (I) or (II) wherein R1 is directly bonded via a N atom, by a Buchwald coupling of a compound of formula (V)

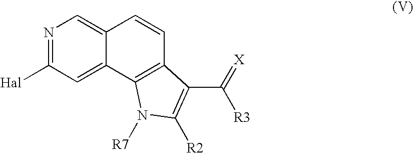

wherein Hal is a halogen, e.g. Cl, and R2, R3 and X are as defined with respect to the corresponding formula (I) or (II),

with a Buchwald coupling reagent compound of formula R1-H wherein the H is part of a —NH₂ group contained within R1, in the presence of suitable reaction conditions to effect coupling.

In both steps (a) and (b), protecting groups may be introduced if necessary prior to the coupling reaction and subsequently removed following the coupling.

The compounds of formula (I) in free form may be converted into salt forms in conventional manner and vice-versa.

The compounds of the invention can be recovered from the reaction mixture and purified in conventional manner. Isomers, such as enantiomers, may be obtained in conventional manner, e.g. by fractional crystallization or asymmetric synthesis from corresponding asymmetrically substituted, e.g. optically active starting materials.

In an eighth aspect the invention provides a combination comprising a compound according to any one of claims 1-7 in combination with one or more active agents selected from the following: Anti IL-1 agents, anti cytokine and anti-cytokine receptor agents, B-cell and T-cell modulating drugs, disease-modifying anti-rheumatic agents (DMARDs), gold salts, penicillamine, hydroxychloroquine and chloroquine, azathioprine, glucocorticoids, non-steroidal anti-inflammatories (NSAIDs), selective COX-2 inhibitors, agents which modulate migration of immune cells, chemokine receptor antagonists, modulators of adhesion molecules, for simultaneous, separate or sequential administration.

Agents of the invention may be prepared by processes described below, which are intended to be non-limiting examples:

Experimental Procedures

Abbreviations:

R(+)-BINAP R-(+)-2,2′-bis(diphenylphosphino)-1,1′-binaphthalene

Boc Pert-butoxycarbonyl

CCl₄ carbon tetrachloride

CH₂Cl₂ methylene chloride

Cu copper

Cs₂CO₃ cesium carbonate

DIPEA N,N-diisopropylethylamine

DMAP 4-dimethylaminopyridine

DMF N,N-dimethylformamide

DMSO dimethyl sulfoxide

dppf (diphenylphosphino)ferrocene

EDC N-(3-dimethylaminopropyl)-N′-ethyl-carbodiimid

EtOAc ethyl acetate

EtOH ethanol

H₂O water

HCl_{conc} concentrated hydrochloric acid (37% in water)

HOBT 1-hydroxybenzotriazol

K₂CO₃ potassium carbonate

MeOH methanol

Na sodium

NaOH sodium hydroxide

Na₂CO₃ sodium carbonate

NaHCO₃ sodium bicarbonate

Na₂SO₄ sodium sulfate

NBS N-bromosuccinimide

NEt₃ triethylamine

NH₃ ammonia

NH_{3conc} concentrated ammonia (25% in water)

NH₂OH.HCl hydroxylamine hydrochloride

OAc acetate

Pd palladium

PPh₃ triphenylphosphine

Rh rhodium

SiO₂ silica

SOCl₂ thionyl chloride

TBME tent-butyl methyl ether

TBTU O-(benzotriazol-1-y1)-N,N,N′,N′-tetramethyluronium tetrafluoroborate

TFA trifluoroacetic acid

THF tetrahydrofuran

General Synthesis Outline

9,10-Dihydro-8H-3,8,10-triaza-pentaleno[2,1-a]naphthalen-7-ones of type 6 with a substituent R in position 2 are obtained from di-BOC-protected analogue 2 by Suzuki, Stille or Buchwald coupling reactions. The coupling reactions deliver—depending upon the reaction conditions—either a mono-BOC-protected analogue 5 or the di-BOC-protected analogue 3. 3 and 5 are deprotected with HClconc to deliver the desired products 6. The desired products 6 are also obtained by performing the Suzuki coupling reaction with unprotected analogue 4. (Scheme 1). 2 is obtained from 1 by treatment with di-tert-butyl dicarbonate. 4 is obtained from 1 by treatment with EDC/HOBt in DMF (Example 4).

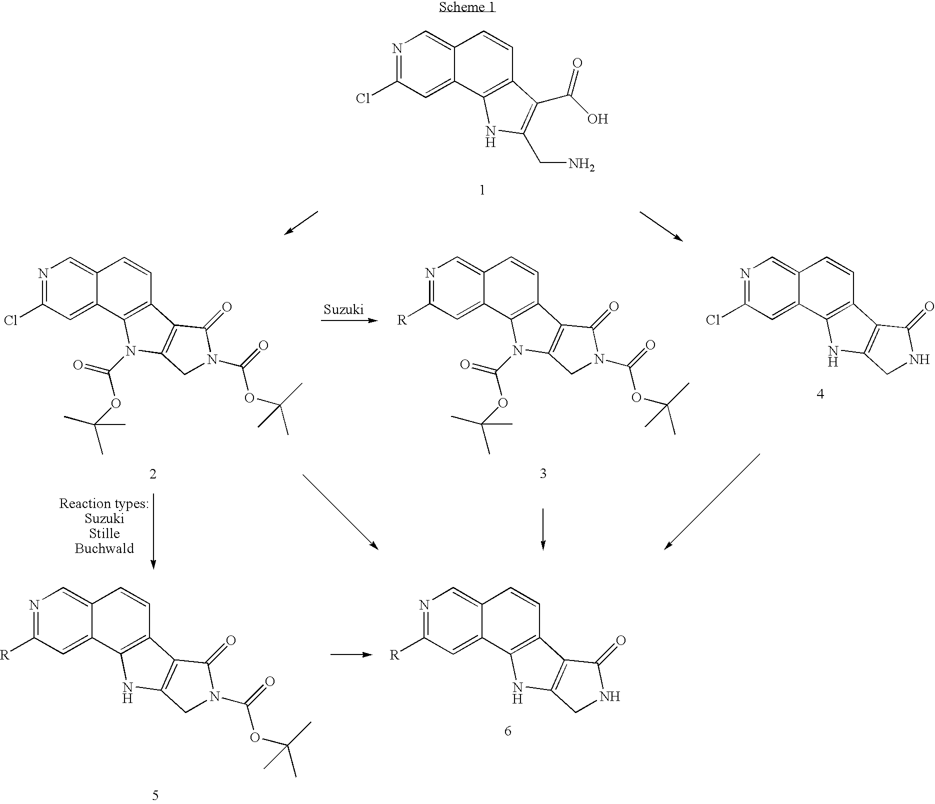

Compound 1 is obtained from 3-chloro-isoquinolin-5-ylamine (US 2004/157849; Scheme 2) in six steps via oxidative cyclisation of enaminone 7 to pyrrolo[2,3-f]isoquinoline 8 in analogy to similar cyclisations described before in the literature (J. Org. Chem. 1980, 45, 2938). The BOC-protected 9 is brominated with NBS to yield 10, which upon treatment with NH₃ and deprotection of 11 delivered 1.

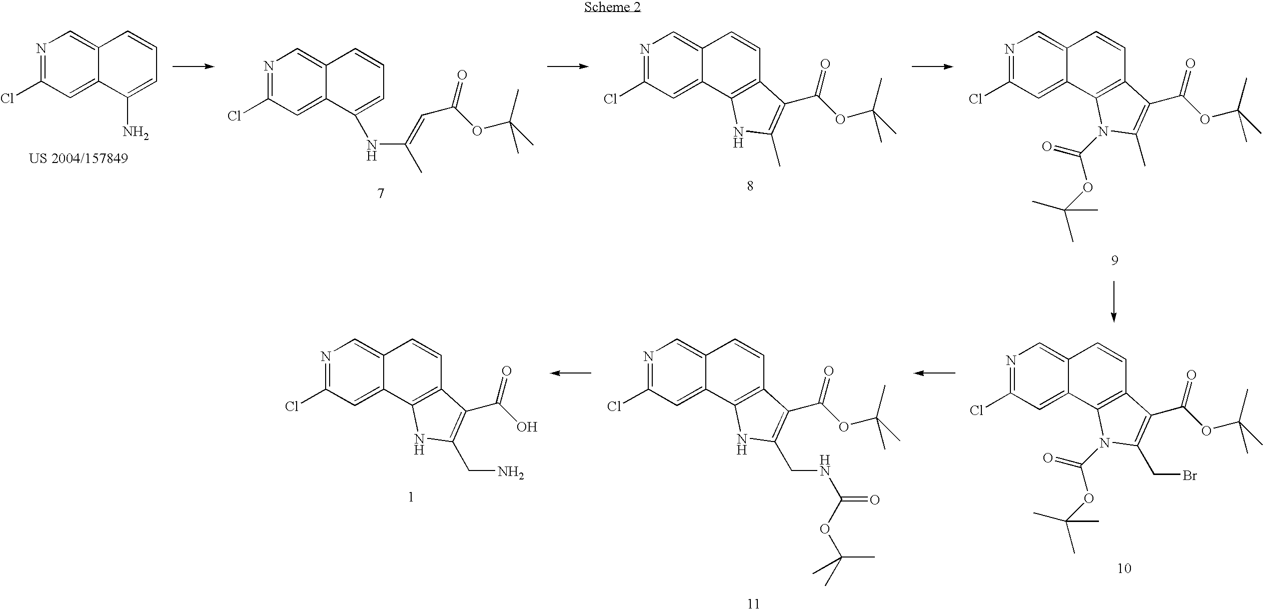

The Br-atom in 10 is converted into an ether (Scheme 3) by reacting with an alcohol ROH. Subsequent Suzuki reaction, deprotection and conversion of the acid to an amide delivered compound 12. 10 is treated with tert-butyl N-hydroxycarbamate, tert-butyl carbazate or hydrazines followed by a Suzuki reaction and provided 13 (X═O, NR). Deprotection and cyclisation delivered 14. Alternatively, the order of cyclisation and Suzuki coupling may be exchanged. Treatment of 10 with amines R′NH₂followed by a Suzuki reaction delivered 15, which upon cyclisation delivered 16. Deprotection of 15 leads to amino acid 22 (Example 1), which can further be modified to deliver esters or amides.

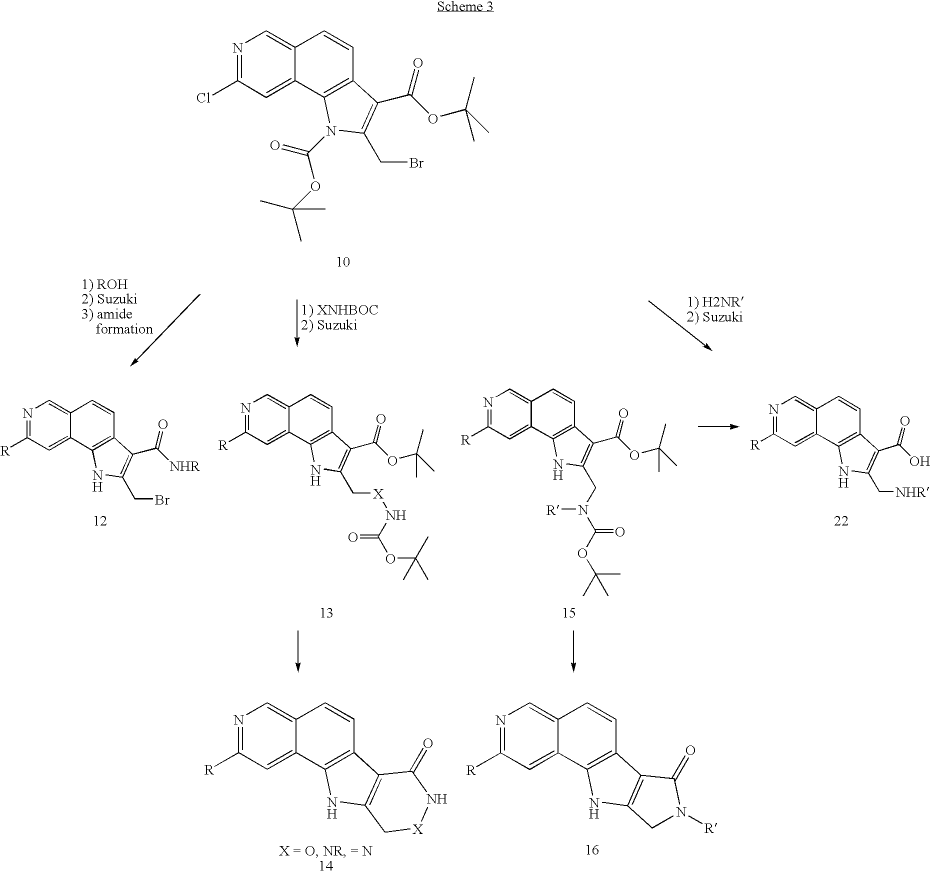

9,10,11,12-Tetrahydro-8H-3,8,12-triaza-naphtho[2,1-a]azulen-7-one analogues 21 (Scheme 4) with substituents R″ in position 2 are prepared from 2-chloro-9,10,11,12-tetrahydro-8H-3,8,12-triaza-naphtho[2,1-a]azulen-7-one 20 via Suzuki or Stifle reactions. 20 is obtained via Beckmann rearrangement from oxime 19. Oxime 19 is obtained from ketone 18, which is prepared via an oxidative cyclisation of enaminone 17 in analogy to similar cyclisations described before in the literature (J. Org. Chem. 1980, 45, 2938). 17 can be prepared from commercially available 5-aminoisoquinoline or from 3-chloro-isoquinolin-5-ylamine (US 2004/157849).

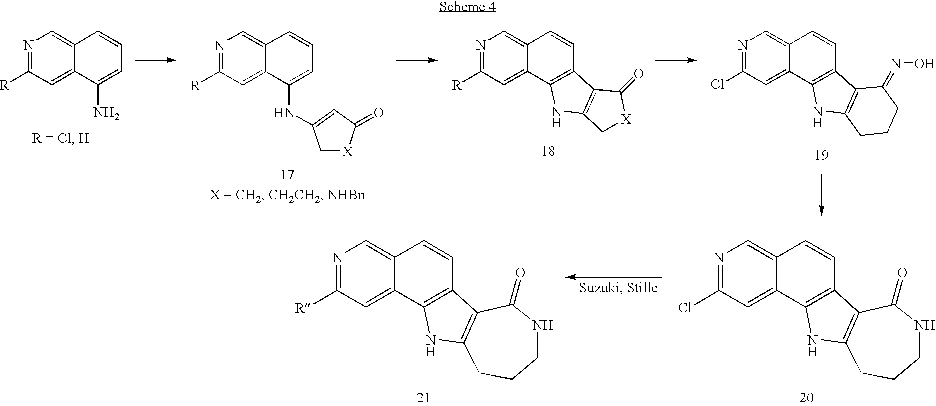

Synthesis of Starting Materials and Intermediates:

### (E)-3-(3-Chloroisoquinolin-5-ylamino)-but-2-enoic acid tert-butyl ester

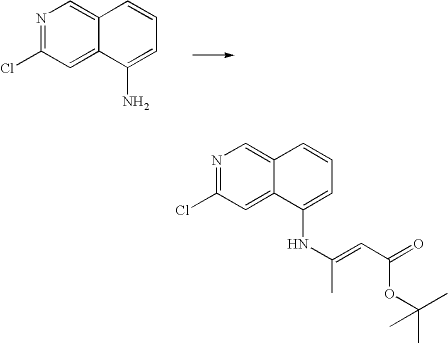

3-Chloroisoquinoline-5-ylamine (US 2004/157849) (2.1 g; 11.8 mmol) is dissolved in 3-oxobutyric acid tert-butyl ester (52 ml), acetic acid (4.2 ml) added and heated at 50° C. for 4 h. The reaction mixture is evaporated and the resulting crystals purified via chromatography (SiO₂, hexanes/acetone 85/15) to yield the title product as yellowish crystals. Triturating the latter with cold hexanes delivers the title product as nearly colorless crystals.

1H-NMR (400 MHz; DMSO-d6): 1.49 (s, 9H); 1.88 (s, 3H); 4.78 (s, 1H); 7.68 (m, 2H); 7.76 (s, 1H); 8.03 (bd, 1H); 9.26 (s, 1H); 10.52 (s, 1H).

MS (m/z) ES+: (319 (MH+).

### 8-Chloro-2-methyl-1H-pyrrolo[2,3-f]isoquinoline-3-carboxylic acid tert-butyl ester

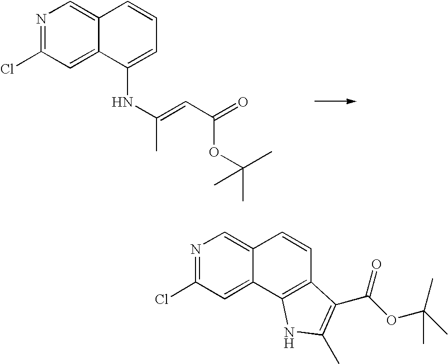

Pd(OAc)₂ (97 mg; 0.43 mmol) and Cu(OAc)₂ (780 mg; 3.9 mmol) are dissolved in DMF (13 ml) at 60° C. and added to a solution of (E)-3-(3-chloroisoquinolin-5-ylamino)-but-2-enoic acid tert-butyl ester (691 mg; 2.17 mmol) in DMF (3 ml). The reaction mixture is heated to 120° C. for 10 min. and evaporated to dryness. The residue is taken up in acetone (20 ml), hexanes (80 ml) added, filtered and the filtrate purified via chromatography (SiO₂; acetone/hexanes 2/8) to yield the title compound as yellowish crystals.

1H-NMR (400 MHz; DMSO-d6): 1.61 (s, 9H); 2.74 (s, 3H); 7.78 (d, 1H); 8.20 (d, 1H); 8.32 (s, 1H); 9.12 (s, 1H); 12.76 (bs, 1H).

MS (m/z) ES+: 317 (MH+); 261 (100).

### 8-Chloro-2-methyl-pyrrolo[2,3-f]isoquinoline-1,3-dicarboxylic acid di-tert-butyl ester

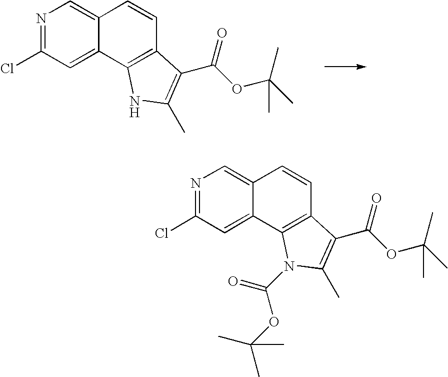

8-Chloro-2-methyl-1H-pyrrolo[2,3-f]isoquinoline-3-carboxylic acid tert-butyl ester (3 g; 9.44 mmol), di-tert-butyl-dicarbonat (7.5 g; 34 mmol) and DMAP (22 mg; 0.094 mmol) are dissolved in diglyme (30 ml) and heated to 120° C. for 10 min. A second portion of di-tert-butyl-dicarbonat (7.5 g; 34 mmol) is added and heating continued for 15 min. A third portion of di-tert-butyl-dicarbonat (7.5 g; 34 mmol) is added and heating continued for another 15 min. The reaction mixture is evaporated and the residue purified by chromatography (SiO₂, hexanes/acetone 1/0 to 9:1) to yield the title compound as yellowish soft crystals.

1H-NMR (400 MHz; DMSO-d6): 1.63 (s, 9H); 1.70 (s, 9H); 2.86 (s, 3H); 7.91 (s, 1H); 8.02 (d, 1H); 8.32 (d, 1H); 9.25 (s, 1H).

MS (m/z) ES+: 417 (MH+, 20); 361 (100); 305 (20).

### 2-Bromomethyl-8-chloro-pyrrolo[2,3-f]isoquinoline-1,3-dicarboxylic acid di-tert-butyl ester

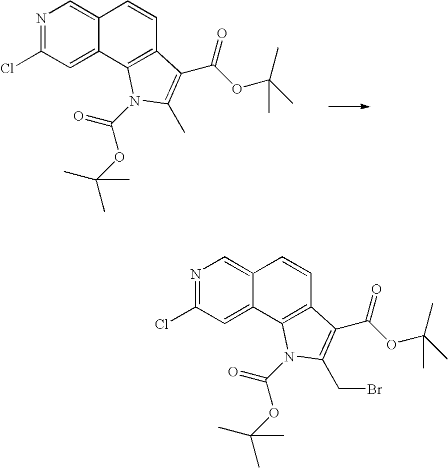

8-Chloro-2-methyl-pyrrolo[2,3-f]isoquinoline-1,3-dicarboxylic acid di-tert-butyl ester (3.78 g; 0.1 mmol) in CCl₄ (25 ml) is combined with NBS (1.78 g; 9.99 mmol) and dibenzoylperoxide (110 mg; 0.455 mmol) and refluxed for 2.5 h. The reaction mixture is evaporated and purified via chromatography (SiO₂; hexanes/acetone 4:1) to render the title compound as yellowish crystals.

1H-NMR (400 MHz; DMSO-d6): 1.67 (s, 9H); 1.75 (s, 9H); 5.36 (s, 2H); 8.00 (s, 1H); 8.10 (d, 1H); 8.37 (d, 1H); 9.31 (s, 1H).

MS (m/z) ES+: 497 (MH+; 10); 495 (8); 441 (100); 439 (70); 385 (10); 383 (8).

### 2-(tert-Butoxycarbonylamino-methyl)-8-chloro-1H-pyrrolo[2,3-f]isoquinoline-3-carboxylic acid tert-butyl ester

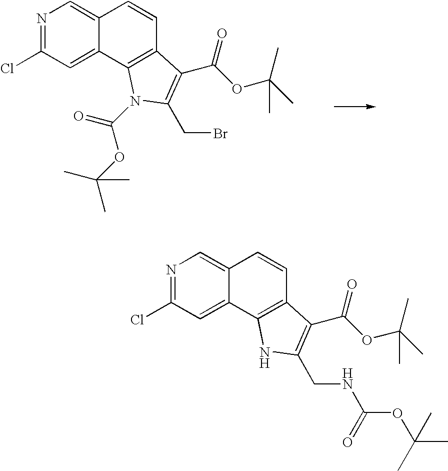

2-Bromomethyl-8-chloro-pyrrolo[2,3-f]isoquinoline-1,3-dicarboxylic acid di-tert-butyl ester (200 mg; 0.4 mmol) is dissolved in dioxane (3 ml) and combined with NH₃conc (2 ml). The reaction mixture is heated in a microwave oven at 100° C. for 10 min. and purified via chromatography (SiO₂; TBME/hexanes 3/7) to yield the title compound as yellow foam.

1H-NMR (400 MHz; DMSO-d6): 1.44 (s, 9H); 1.62 (s, 9H); 4.72 (d, 2H); 7.15 (bs, 1H); 7.81 (d, 1H); 8.23 (d, 1H); 8.66 (s, 1H); 9.13 (s, 1H); 12.69 (bs, 1H).

MS (m/z) ES+: 432 (MH+); 376 (20).

### 2-(tert-Butoxycarbonylamino-methyl)-8-((E)-styryl)-1H-pyrrolo[2,3-f]isoquinoline-3-carboxylic acid tert-butyl ester

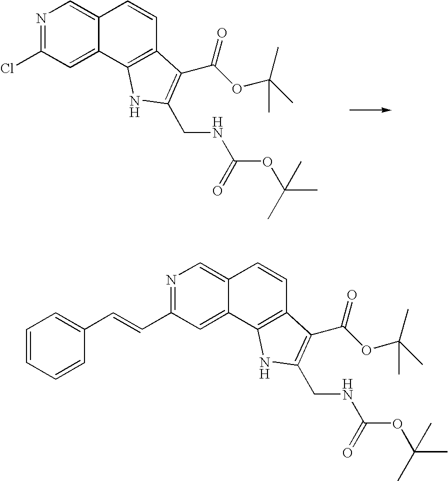

2-(tert-Butoxycarbonylamino-methyl)-8-chloro-1H-pyrrolo[2,3-f]isoquinoline-3-carboxylic acid tert-butyl ester (139 mg; 0.322 mmol), trans-phenylvinyl boronic acid (143 mg; 0.96 mmol), K₂CO₃ (58 mg; 0.42 mmol) and Pd(dppf)₂Cl₂ (66 mg; 0.08 mmol) are combined in DMF/water (5 ml/2 ml) and heated at 90° C. for 1.5 h. The reaction mixture is poured on brine and extracted with TBME three times. The combined organic phases are dried over Na₂SO₄, filtered, evaporated to dryness and purified via chromatography (SiO₂, EtOAc/hexanes 2/8) to yield the title compound as yellowish foam.

1H-NMR (400 MHz; DMSO-d6): 1.44 (s, 9H); 1.63 (s, 9H); 4.74 (d, 2H); 7.12 (bs, 1H); 7.30-7.45 (m, 4H); 7.65-7.85 (m, 4H); 8.17 (d, 1H); 8.48 (s, 1H); 9.25 (s, 1H); 12.69 (bs, 1H).

MS (m/z) ES+: 500 (MH+).

### EXAMPLE 1

### b 2-Aminomethyl-8-((E)-styryl)-1H-pyrolo[2,3-f]isoquinoline-3-carboxylic acid hydrochloride

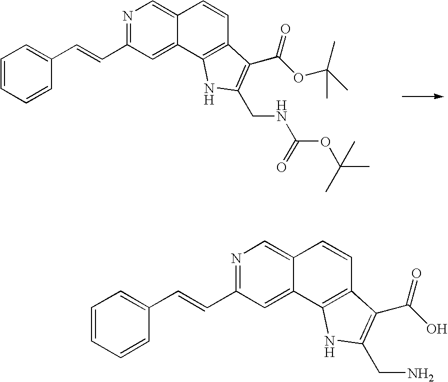

2-(tert-Butoxycarbonylamino-methyl)-8-((E)-styryl)-1H-pyrrolo[2,3-f]isoquinoline-3-carboxylic acid tert-butyl ester (72 mg; 0.14 mmol) is dissolved in HClconc (2 ml). After 3 min at room temperature the reaction mixture is evaporated to dryness to yield the title compound as yellow crystals.

1H-NMR (400 MHz; DMSO-d6): 4.58 (bd, 2H); 7.40 (m, 1H); 7.48 (m, 3H); 7.71 (m, 2H); 7.87 (d, 1H); 8.00 (d, 1H); 8.35 (d, 1H); 8.50 (bs, 3H); 8.71 (bs, 1H); 9.55 (s, 1H); 14.12 (bs, 1H).

MS (m/z) ES+: 344 (MH+).

### EXAMPLE 2

### 2-((E)-Styryl)-9,10-dihydro-8H-3,8,10-triaza-pentaleno[2,1-a]naphthalen-7-one

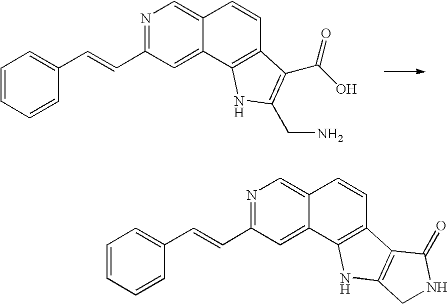

2-Aminomethyl-8-((E)-styryl)-1H-pyrrolo[2,3-f]isoquinoline-3-carboxylic acid hydrochloride (50 mg; 0.13 mmol) and HOBt (20 mg; 0.13 mmol) are suspended in DMF (10 ml). N-(3-dimethylaminopropyl)-N′-ethyl-carbodiimid (EDC; 41 mg; 0.26 mmol) is added and the resulting solution left over night at room temperature. The reaction mixture is taken up in CH₂Cl₂ and purified via chromatography (SiO₂; TBME/MeOH/NH₃conc 93/7/0.4 to 90/10/0.4) to yield the title compound as yellowish solid.

1H-NMR (400 MHz; DMSO-d6): 4.54 (s, 2H); 7.32 (m, 1H); 7.42 (m, 3H); 7.71 (bd, 2H); 7.81 (m, 3H); 7.86 (d, 1H); 8.28 (s, 1H); 9.29 (s, 1H); 13.02 (bs, 1H).

MS (m/z) ES+: 326 (MH+).

### EXAMPLE 3

### 2-Aminomethyl-8-chloro-1H-pyrrolo[2,3-f]isoquinoline-3-carboxylic acid hydrochloride

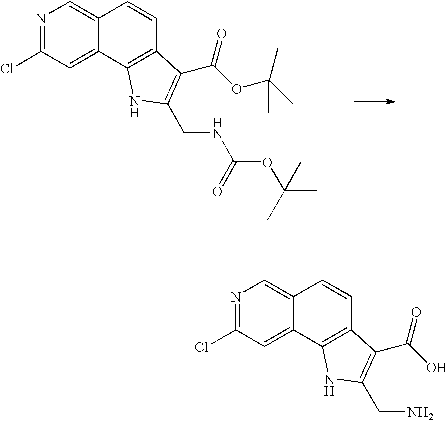

2-(tert-Butoxycarbonylamino-methyl)-8-chloro-1H-pyrrolo[2,3-f]isoquinoline-3-carboxylic acid tert-butyl ester (90 mg; 0.21 mmol) is dissolved in HClconc (1 ml) and kept at room temperature for 2 min. The reaction mixture is evaporated and the title compound isolated as a yellowish solid which is used for the next step without purification.

1H-NMR (400 MHz; DMSO-d6): 4.55 (bs, 2H); 4.75 (bs 1H); 7.86 (d, 1H); 8.27 (d, 1H); 8.48 (s, 1H); 8.52 (bs, 2H); 9.19 (s, 1H); 13.90 (s, 1H).

MS (m/z) ES−: 274 (MH−).

### EXAMPLE 4

### 2-Chloro-9,10-dihydro-8H-3,8,10-triaza-pentaleno[2,1-a]naphthalen-7-one

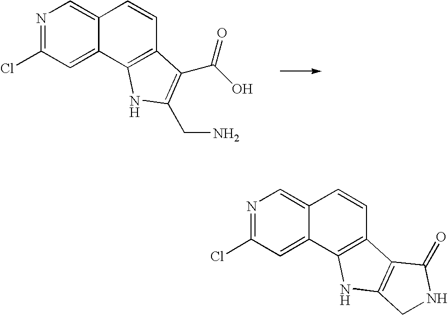

2-Aminomethyl-8-chloro-1H-pyrrolo[2,3-f]isoquinoline-3-carboxylic acid hydrochloride (102 mg; 0.32 mmol) and HOBt (50 mg; 0.32 mmol) are suspended in DMF (15 ml) and combined with N-(3-dimethylaminopropyl)-N′-ethyl-carbodiimid (EDC; 101 mg; 0.655 mmol). The resulting solution is heated to 55° C. for 15 min. and left at room temp. over night. The reaction mixture is evoporated to dryness and purified via chromatography (SiO₂; TBME/MeOH/NH₃conc 90/10/0.4 to 85/15/0.4) to deliver the title compound as yellowish crystals.

1H-NMR (400 MHz; DMSO-d6): 4.55 (s, 2H); 7.82 (d, 1H); 7.92 (d, 1H); 8.34 (s, 1H); 9.17 (s, 1H); 13.03 (bs, 1H).

MS (m/z) ES−: 256 (MH−).

### EXAMPLE 5

### 2-Aminooxymethyl-8-((E)-styryl)-1H-pyrolo[2,3-f]isoquinoline-3-carboxylic acid hydrochloride

### 2-tert-Butyloxycarbonyl-aminooxymethyl-8-chloro-1H-pyrrolo[2,3-f]isoquinoline-3-carboxylic acid tert-butyl ester

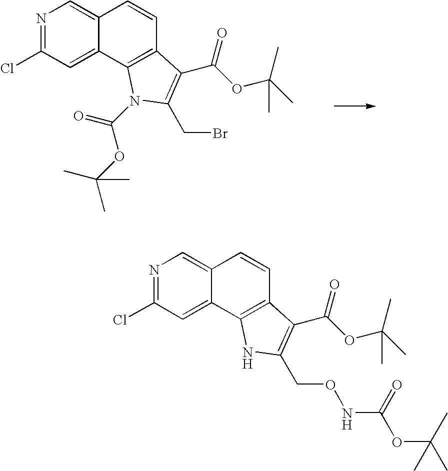

2-Bromomethyl-8-chloro-pyrrolo[2,3-f]isoquinoline-1,3-dicarboxylic acid di-tert-butyl ester (200 mg; 0.4 mmol), tert-butyl-N-hydroxycarbamat (538 mg; 4 mmol) and K₂CO₃ (47 mg; 3.2 mmol) are dissolved in 1,4-dioxane (20 ml) and refluxed for 15 min. The reaction mixture is poured on brine and extracted with TBME three times. The combined organic phases are washed with 2N NaOH, dried over Na₂SO₄, filtered and evaporated to dryness. The resulting brown foam is crystallised form TBME to deliver the title compound as off-white crystals.

1H-NMR (400 MHz; DMSO-d6): 1.35 (s, 9H); 1.61 (s, 9H); 5.32 (s, 2H); 7.82 (d, 1H); 8.25 (d, 1H); 8.59 (s, 1H); 9.15 (s, 1H); 10.15 (bs, 1H); 13.00 (bs, 1H).

MS (m/z) ES+: 448 (MH+).

### 2-tert-Butyloxycarbonyl-aminooxymethyl-8-((E)-styryl)-1H-pyrrolo[2,3-f]isoquinoline-3-carboxylic acid tert-butyl ester

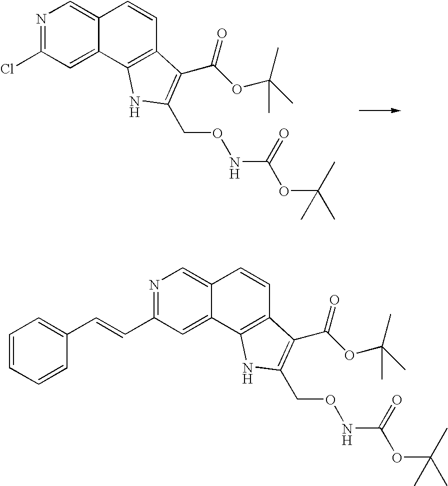

2-tert-Butyloxycarbonyl-aminooxymethyl-8-chloro-1H-pyrrolo[2,3-f]isoquinoline-3-carboxylic acid tert-butyl ester (60 mg; 0.13 mmol)), trans-phenylvinyl boronic acid (40 mg; 0.27 mmol), K₂CO₃ (28 mg; 0.21 mmol) and Pd(dppf)₂Cl₂ (27 mg; 0.03 mmol) are combined in DMF/water (2.5 ml/1 ml) and heated at 90° C. for 4 h. The reaction mixture is poured on water and extracted with TBME three times. The combined organic phases are dried over Na₂SO₄, filtered, evaporated to dryness and purified via chromatography (SiO₂, acetone/hexanes 8/92) to yield the title compound as yellow crystals.

1H-NMR (400 MHz; DMSO-d6): 1.37 (s, 9H); 1.62 (s, 9H); 5.33 (s, 2H); 7.30-7.38 (m, 1H); 7.41-7.47 (m, 3H); 7.70 (bd, 2H); 7.76 (d, 1H); 7.79 (d, 1H); 8.21 (d, 1H); 8.52 (s, 1H); 9.26 (s, 1H); 10.17 (bs, 1H); 13.00 (bs, 1H).

MS (m/z) ES+: 516 (MH+).

### 2-Aminooxymethyl-8-((E)-styryl)-1H-pyrrolo[2,3-f]isoquinoline-3-carboxylic acid hydrochloride

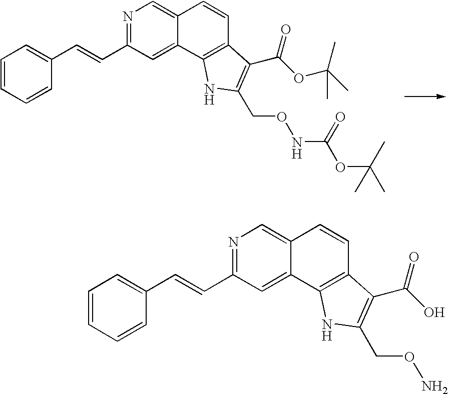

2-tert-Butyloxycarbonyl-aminooxymethyl-8-((E)-styryl)-1H-pyrrolo[2,3-f]isoquinoline-3-carboxylic acid tert-butyl ester (25 mg; 0.048 mmol) is dissolved in HClconc (1 ml) and evaporated below 30° C. to dryness to deliver the title compound as yellowish solid.

MS (m/z) ES+: 360 (MH+).

### EXAMPLE 6

### 2-((E)-Styryl)-10,11-dihydro-9-oxa-3,8,11-triaza-benzo[a]fluoren-7-one

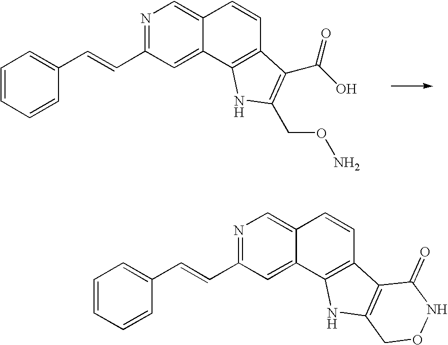

2-Aminooxymethyl-8-((E)-styryl)-1H-pyrrolo[2,3-f]isoquinoline-3-carboxylic acid hydrochloride (20 mg; 0.04 mmol) and HOBt (7.7 mg; 0.05 mmol) are suspended in DMF (2 ml). N-(3-dimethylaminopropyl)-N′-ethyl-carbodiimid (EDC; 16 mg; 0.1 mmol) is added and the resulting solution left at room temperature for 3.5h. The reaction mixture is taken up in CH₂Cl₂ and purified via chromatography (SiO₂; acetone/hexanes 3/7) to yield the title compound as yellow solid.

1H-NMR (400 MHz; DMSO-d6): 5.30 (s, 2H); 7.33 (m, 1H); 7.43 (m, 3H); 7.72 (bd, 2H); 7.81 (m, 2H); 8.08 (d, 1H); 8.25 (s, 1H); 9.31 (s, 1H); 10.35 (s, 1H); 13.13 (s, 1H).

MS (m/z) ES+: 342 (MH+).

### EXAMPLE 7

### 2-Chloro-8,9,10,11-tetrahydro-pyrido[4,3-a]carbazol-7-one

### 3-(3-Chloro-isoquinolin-5-ylamino)-cyclohex-2-enone

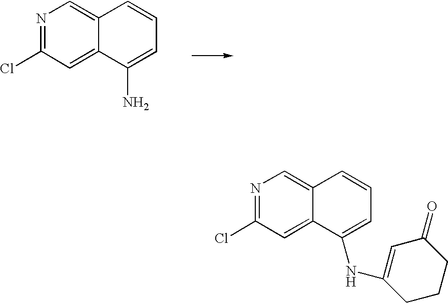

3-Chloroisoquinoline-5-ylamine (U.S. Pat. No. 3,930,837) (200 mg; 1.1 mmol) and 1,3-cyclohexanedione (151 mg; 1.3 mmol) are dissolved in CH₂Cl₂/MeOH (6 ml/0.5 ml), evaporated to dryness and the resulting mixture heated at 120° C. for 20 min. 3-(3-Chloro-isoquinolin-5-ylamino)-cyclohex-2-enone is not purified further and used in the next step.

### 2-Chloro-8,9,10,11-tetrahydro-pyrido[4,3-a]carbazol-7-one

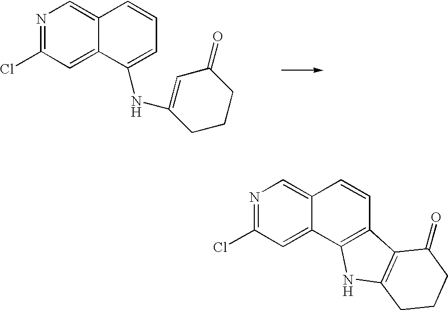

Pd(OAc)₂ (50 mg; 0.22 mmol) and Cu(OAc)₂ . H₂O (400 mg; 2.2 mmol) are dissolved in DMF (6 ml) at 60° C. and added to a solution of 3-(3-chloro-isoquinolin-5-ylamino)-cyclohex-2-enone (350 mg; 2 mmol) in DMF (2 ml). The reaction mixture is heated to 120° C. for 25 min. under argon, cooled to room temp., diluted with CH₂Cl₂, filtered and the filtrate purified via chromatography (SiO₂; acetone/hexanes 3/7) to yield the title compound as brownish crystals.

1H-NMR (400 MHz; DMSO-d6): 2.19 (m, 2H); 2.52 (m, 2H); 3.10 (m, 2H); 7.82 (d, 1H); 8.24 (d, 1H); 8.33 (s, 1H); 9.15 (s, 1H); 12.92 (bs, 1H).

MS (m/z) ES+: 271 (MH+).

### EXAMPLE 8

### 2-Chloro-8,9,10,11-tetrahydro-pyrido[4,3-a]carbazol-7-one oxime

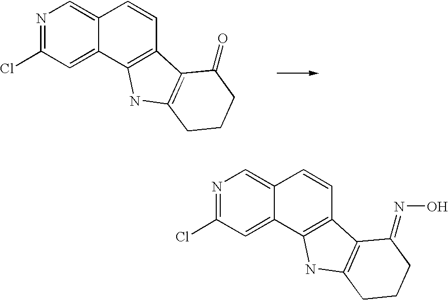

2-Chloro-8,9,10,11-tetrahydro-pyrido[4,3-a]carbazol-7-one (188 mg; 0.7 mmol), NH2OH.HCl (188 mg; 2.7 mmol) and pyridine (188 mg; 2.4 mmol) are dissolved in EtOH (15 ml) and refluxed for 1.5 h. The reaction mixture is evaporated to a volume of ˜6 ml and water (8 ml) added dropwise. The resulting precipitate is filtered and dried to yield the title compound.

1H-NMR (400 MHz; DMSO-d6): 1.97 (m, 2H); 2.74 (bt, 2H); 2.94 (bt, 2H); 7.69 (d, 1H); 8.19 (d, 1H); 8.29 (s, 1H); 9.09 (s, 1H); 10.47 (s, 1H); 12.42 (bs, 1H).

MS (m/z) ES+: 286 (MH+).

### EXAMPLE 9

### 2-Chloro-9,10,11,12-tetrahydro-8H-3,8,12-triaza-naphtho[2,1-a]azulen-7-one

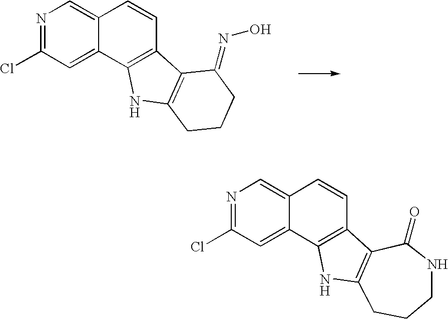

2-Chloro-8,9,10,11-tetrahydro-pyrido[4,3-a]carbazol-7-one oxime (200 mg; 0.7 mmol) is suspended in 1,4-dioxane (5 ml) and added to polyphosphoric acid (6 g). The reaction mixture is heated to 110° C. for 20 min. The reaction mixture is poured on water, pH adjusted to ˜11 by the addition of solid Na₂CO₃. The product precipitates from the aq. phase, is filtered, washed with acetone, dried and the title compound is obtained as yellowish crystals.

MS (m/z) ES+: 286 (MH+).

### EXAMPLE 10

### 2-((E)-Styryl)-9,10,11,12-tetrahydro-8H-3,8,12-triaza-naphtho[2,1-a]azulen-7-one

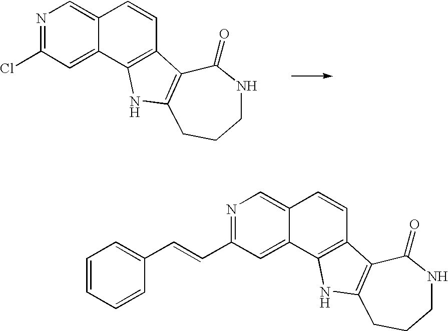

2-Chloro-9,10,11,12-tetrahydro-8H-3,8,12-triaza-naphtho[2,1-a]azulen-7-one (20 mg; 0.07 mmol), trans-phenylvinyl boronic acid (40 mg; 0.27 mmol), NaOH 2N (0.14 ml; 0.28 mmol), Pd(PPh₃)₂Cl₂(15 mg; 0.02 mmol), PPh₃(33 mg; 0.126 mmol) are dissolved in DMF (3 ml) and heated to 140° C. for 2 h. The reaction mixture is evaporated, purified via chromatography (SiO2; acetone/hexanes 7/3-8/2) to yield a yellow foam, which crystallized with TBME to deliver the title compound as pale yellow crystals.

1H-NMR (400 MHz; DMSO-d6): 2.09 (m, 2H); 3.29 (m, 4H); 7.26-7.45 (m, 4H); 7.59 (m, 1H); 7.70-7.80 (m, 4H); 8.26 (s, 1H); 8.46 (d, 1H); 9.24 (s, 1H); 12.54 (s, 1H).

MS (m/z) ES+: 354 (MH+).

### EXAMPLE 11

### 2-(4-Fluoro-phenyl)-9,10,11,12-tetrahydro-8H-3,8,12-triaza-naphtho[2,1-a]azulen-7-one

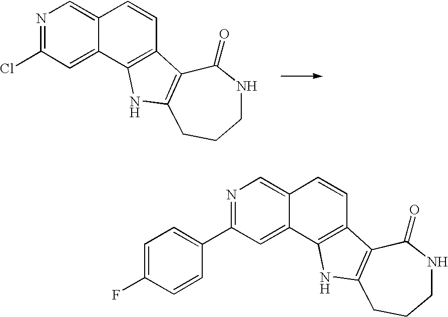

2-Chloro-9,10,11,12-tetrahydro-8H-3,8,12-triaza-naphtho[2,1-a]azulen-7-one (25 mg; 0.087 mmol), 4-fluorophenylboronic acid (49 mg; 0.36 mmol), NaOH 2N (0.18 ml; 0.35 mmol), PPh₃ (41 mg; 0.16 mmol) are dissolved in DMF (3 ml) and combined with Pd(PPh₃)₂Cl₂(18 mg; 0.026 mmol) dissolved in DMF (2 ml). The reaction mixture is heated to 140° C. for 9 h. The reaction mixture is evaporated, purified via chromatography (SiO2; acetone/hexanes 7/3-8/2) to yield a yellow foam, which is washed with cold acetone and delivers the title compound as yellow solid.

1H-NMR (400 MHz; DMSO-d6): 2.09 (s, 4H); 3.27 (m, 2H); 7.39 (m, 2H); 7.59 (m, 1H); 7.68 (d, 1H); 8.24 (m, 2H); 8.46 (d, 1H); 8.81 (s, 1H); 9.29 (s, 1H); 12.55 (bs, 1H).

MS (m/z) ES+: 346 (MH+).

### EXAMPLE 12

### 2-Aminooxymethyl-8-chloro-1H-pyrolo[2,3-f]isoquinoline-3-carboxylic acid hydrochloride

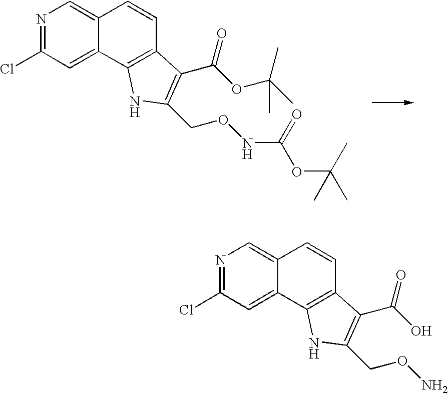

2-tert-Butyloxycarbonyl-aminooxymethy1-8-chloro-1H-pyrrolo[2,3-f]isoquinoline-3-carboxylic acid tert-butyl ester (43 mg; 0.096 mmol) is dissolved in HClconc at room temperature. After two minutes the reaction mixture is evaporated to dryness and yields the desired compound as yellow solid.

1H-NMR (400 MHz; DMSO-d6): 5.13 (bs, 2H); 5.64 (s, 2H); 7.84 (d, 1H); 8.29 (d, 1H); 8.81 (s, 1H); 9.81 (s, 1H); 11.14 (bs, 1H); 13.46 (bs, 1H).

MS (m/z) ES+: 292 (MH+; 50); 259 (100).

### EXAMPLE 13

### 2-Chloro-10,11-dihydro-9-oxa-3,8,11-triaza-benzo[a]fluoren-7-one

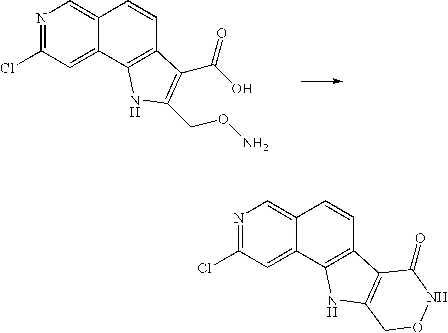

2-Aminooxymethyl-8-chloro-1H-pyrrolo[2,3-f]isoquinoline-3-carboxylic acid hydrochloride (31 mg; 0.094 mmol)) and HOBt.H₂O (15 mg; 0.094 mmol) are suspended in DMF (2 ml). N-(3-dimethylaminopropyl)-N′-ethyl-carbodiimid (EDC; 29 mg; 0.19 mmol) is added and the resulting solution kept at room temperature for 1 h, evaporated and purified via chromatography (SiO2; acetone/hexanes 3/7) to deliver the title compound as colorless crystals.

1H-NMR (400 MHz; DMSO-d6): 5.31 (s, 2H); 7.87 (d, 1H); 8.11 (d, 1H); 8.34 (s, 1H); 9.18 (s, 1H); 10.40 (s, 1H); 13.11 (bs, 1H).

MS (m/z) ES+: 274 (MH+).

### EXAMPLE 14

### 8-Chloro-2-hydrazinomethyl-1H-pyrrolo[2,3-f]isoquinoline-3-carboxylic acid hydrochloride

### 2-(N′Cert-Butoxycarbonyl-hydrazinomethyl)-8-chloro-1H-pyrrolo[2,3-f]isoquinoline-3-carboxylic acid tert-butyl ester

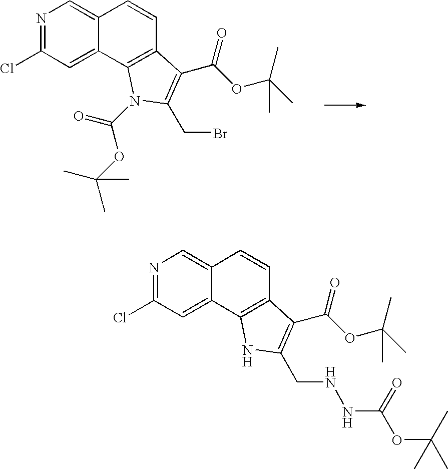

2-Bromomethyl-8-chloro-pyrrolo[2,3-f]isoquinoline-1,3-dicarboxylic acid di-tert-butyl ester (200 mg; 0.4 mmol) in EtOH (8 ml) is added dropwise under stirring to a solution of H₂NNH₂.H₂O (1 ml; 20 mmol) in EtOH (20 ml). Stirring is continued for 15 min., the reaction mixture poured on water and extracted with TBME three times. The combined organic phases are dried over Na₂SO₄, filtered, evaporated to dryness to deliver the title compound as yellow foam.

1H-NMR (400 MHz; DMSO-d6): 1.40 (s, 9H); 1.61 (s, 9H); 4.77 (bs, 2H); 5.07 (s, 2H); 7.82 (d, 1H); 8.23 (d, 1H); 8.65 (s, 1H); 9.14 (s, 1H; 12.26 (bs, 1H).

MS (m/z) ES+: 447 (MH+).

### 8-Chloro-2-hydrazinomethyl-1H-pyrrolo[2,3-f]isoquinoline-3-carboxylic acid hydrochloride

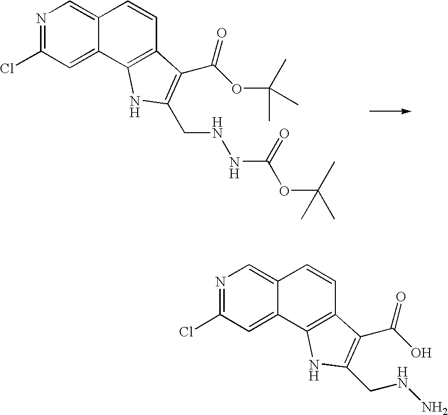

2-(N′-tert-Butoxycarbonyl-hydrazinomethyl)-8-chloro-1H-pyrrolo[2,3-f]isoquinoline-3-carboxylic acid tert-butyl ester (180 mg; 0.4 mmol) is dissolved in HClconc (1 ml), kept 1-2 min at room temperature and evaporated to dryness. The resulting solid is washed with MeOH to yield the title compound as off-white solid.

1H-NMR (400 MHz; DMSO-d6): 3.73 (bs, 3H); 4.65 (s, 2H); 7.82 (d, 1H); 8.27 (d, 1H); 8.64 (s, 1H); 9.15 (s, 1H); 13.40 (s, 1H).

MS (m/z) ES−: 289(MH−).

### EXAMPLE 15 AND 16

### 2-Chloro-8,11-dihydro-3,8,9,11-tetraaza-benzo[a]fluoren-7-one and 2-chloro-8,9,10,11-tetrahydro-3,8,9,11-tetraaza-benzo[a]fluoren-7-one

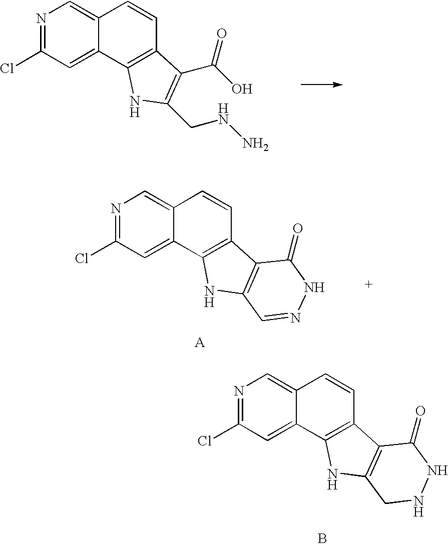

8-Chloro-2-hydrazinomethyl-1H-pyrrolo[2,3-f]isoquinoline-3-carboxylic acid hydrochloride (94 mg; 0.28 mmol), HOBt (44 mg; 0.28 mmol) and N-(3-dimethylaminopropyl)-N′-ethyl-carbodiimid (EDC; 89 mg; 0.57 mmol) are dissolved in DMF (10 ml) and stirred over night. The reaction mixture is evaporated and purified via chromatography (SiO2; TBME/MeOH/NH₃conc 90/10/0.6) to deliver the title compound B as colorless crystals and compound A.

Compound A. 1H-NMR (400 MHz; DMSO-d6): 7.97 (d, 1H); 8.37 (d, 1H); 8.55 (s, 2H); 9.30 (s, 1H); 12.78 (s, 1H); 13.45 (bs, 1H).

MS (m/z) ES−: 269 (MH−).

Compound B. 1H-NMR (400 MHz; DMSO-d6): 4.19 (d, 2H); 5.68 (bt, 1H); 7.78 (d, 1H); 8.13 (d, 1H); 8.31 (s, 1H); 8.55 (s, 1H); 9.15 (s, 1H); 12.80 (bs, 1H).

MS (m/z) ES−: 271 (MH−).

### EXAMPLE 17

### 2-(4-Methoxy-phenyl)-9-methyl-8,9,10,11-tetrahydro-3,8,9,11-tetraaza-benzo[a]fluoren-7-one

### 8-Chloro-2-(N-methyl-hydrazinomethyl)-benzo[g]indole-1,3-dicarboxylic acid di-tert-butyl ester

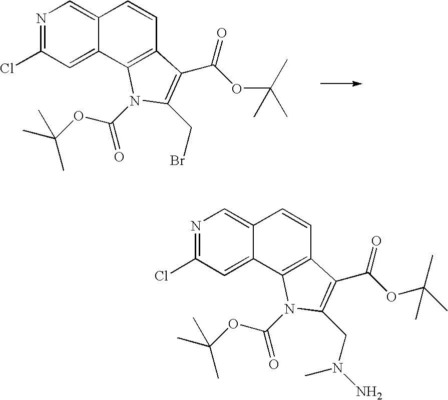

2-Bromomethyl-8-chloro-pyrrolo[2,3-f]isoquinoline-1,3-dicarboxylic acid di-tert-butyl ester (200 mg; 0.4 mmol), methyl hydrazine (46.5 mg; 1 mmol) and NaHCO₃ (51 mg; 0.6 mmol) are dissolved in 1,4-dioxane (5 ml) and refluxed for 3 hours. The reaction mixture is poured on aq. NaHCO₃ and extracted with ethylacetate. After drying and evaporation of the solvent the title compound is purified by reversed phase HPLC.

1H-NMR (400 MHz; DMSO-d6): 1.32 (s, 9H), 1.64 (s, 9H), 2.77 (s, 3H), 4.45 (s, 2H), 7.81 (d, 1H), 8.26 (d, 1H), 8.41 (s, 1H), 9.18 (s, 1H).

MS (m/z) ES+: 461 (MH⁺)

### 2-Chloro-9-methyl-8,9,10,11-tetrahydro-3,8,9,11-tetraaza-benzo[a]fluoren-7-one

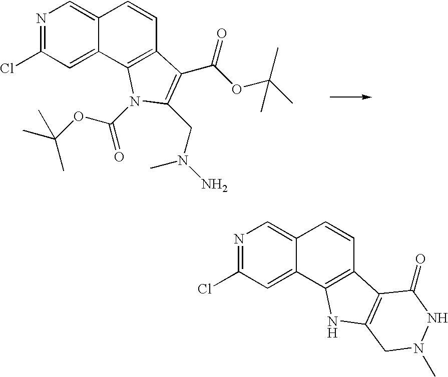

8-Chloro-2-(N-methyl-hydrazinomethyl)-benzo[g]indole-1,3-dicarboxylic acid di-tert-butyl ester (248 mg, 0.5 mmol) is stirred in 4N HCl in 1,4-dioxane (3 ml) at room temperature for 5 hours. The reaction mixture is evaporated and re-dissolved in DMF (2.5 ml). Triethylamine (0.15 ml, 1.6 mmol), HOBt (80 mg, 0.6 mmol) and EDCl (113 mg, 0.6 mmol) are added and the mixture is stirred at room temperature for 16 hours. The mixture is poured onto NaHCO₃ solution and extracted with ethyl acetate. The crude product obtained after evaporation of the solvent is used in the following reaction without further purification.

MS (m/z) ES+: 287 (MH⁺)

### 2-(4-Methoxy-phenyl)-9-methyl-8,9,10,11-tetrahydro-3,8,9,11-tetraaza-benzo[a]fluoren-7-one

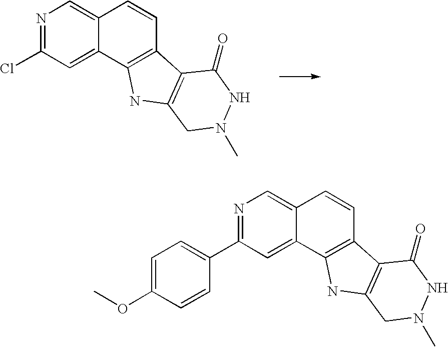

Following the procedure described in example 74 the title compound is obtained by reacting 2-chloro-9-methyl-8,9,10,11-tetrahydro-3,8,9,11-tetraaza-benzo[a]fluoren-7-one and 4-methoxyphenylboronic acid.

1H-NMR (400 MHz; DMSO-d6): 2.62 (s, 3H), 3.85 (s, 3H), 4.40 (s, 2H), 7.13 (d, 2H), 7.77 (d, 1H), 8.08 (d, 1H), 8.17 (d, 2H), 8.75 (s, 1H), 8.79 (s, 1H), 9.33 (s, 1H), 12.9 (bs, 1H).

MS (m/z) ES+: 359 (MH⁺)

### EXAMPLE 18

### 2-(4-Methoxyphenyl)-9,10,11,12-tetrahydro-8H-3,8,12-triaza-naphtho[2,1-a]azulen-7-one

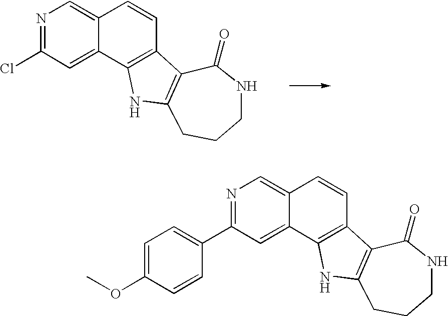

2-Chloro-9,10,11,12-tetrahydro-8H-3,8,12-triaza-naphtho[2,1-a]azulen-7-one (25 mg; 0.087 mmol), 4-methoxyphenylboronic acid (53 mg; 0.35 mmol), NaOH 2N (0.18 ml; 0.35 mmol), PPh₃(41 mg; 0.16 mmol) are dissolved in DMF (3 ml) and combined with Pd(PPh₃)₂Cl₂(18 mg; 0.026 mmol) dissolved in DMF (2 ml). The reaction mixture is heated to 140° C. for 2.5 h. The reaction mixture is evaporated, purified via chromatography (SiO2; acetone/hexanes 8/2) to yield a yellow foam, which is triturated with TBME and recrystallised form acetone to yield the title compound as yellow solid.

1H-NMR (400 MHz; DMSO-d6): 2.09 (s, 4H); 3.27 (m, 2H); 3.85 (s, 3H); 7.12 (m, 2H); 7.58 (bs, 1H); 7.65 (d, 1H); 8.15 (m, 2H); 8.42 (d, 1H); 8.73 (s, 1H); 9.26 (s, 1H); 12.51 (bs, 1H).

MS (m/z) ES+: 358 (MH+).

### EXAMPLE 19

### 2-Methoxymethyl-8-((E)-styryl-1H-pyrrolo[2,3-f]isoquinoline-3-carboxylic acid amide

### 8-Chloro-2-methoxymethyl-1H-pyrrolo[2,3-f]isoquinoline-3-carboxylic acid tert-butyl ester

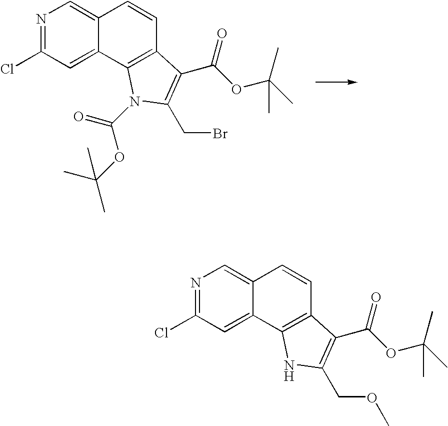

2-Bromomethyl-8-chloro-pyrrolo[2,3-f]isoquinoline-1,3-dicarboxylic acid di-tert-butyl ester (300 mg; 0.607 mmol) is added to a solution of Na (42 mg; 1.8 mmol) in MeOH (3 ml) and refluxed for 70 min. The reaction mixture is poured on water and extracted with TBME three times. The combined organic phases are dried over Na₂SO₄, filtered, evaporated to dryness and purified via chromatography (SiO2; hexanes/acetone 85/15) to yield the title compound as yellowish foam.

1H-NMR (400 MHz; DMSO-d6): 1.61 (s, 9H); 3.46 (s, 3H); 4.99 (s, 2H); 7.80 (d, 1H); 8.23 (d, 1H); 8.69 (s, 1H); 9.13 (s, 1H); 12.92 (bs, 1H).

MS (m/z) ES+: 347 (MH+).

### 2-Methoxymethyl-8-((E)-styryl)-1H-pyrrolo[2,3-f]isoquinoline-3-carboxylic acid tert-butyl ester

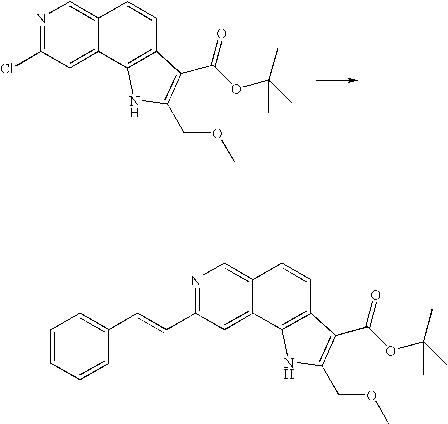

8-Chloro-2-methoxymethyl-1H-pyrrolo[2,3-f]isoquinoline-3-carboxylic acid tert-butyl ester (61 mg; 0.176 mmol), trans-phenylvinyl boronic acid (40 mg; 0.26 mmol), K₂CO₃ (30 mg; 0.22 mmol), Pd(dppf)₂Cl₂ (36 mg; 0.044 mmol) are dissolved in DMF (2 ml)/water (0.75 ml) and heated to 90° C. for 1.5 h. The reaction mixture is poured on water and extracted with EtOAc three times. The combined organic phases are dried over Na₂SO₄, filtered, evaporated to dryness and purified via chromatography (SiO2; hexanes/EtOAc 3/1) to yield the title compound as yellowish foam.

1H-NMR (400 MHz; DMSO-d6): 1.62 (s, 9H); 3.47 (s, 3H); 5.01 (s, 2H); 7.32 (m, 3H); 7.43 (m, 2H); 7.69 (bd, 2H); 7.77 (m, 1H); 8.20 (d, 1H); 8.63 (s, 1H); 9.25 (s, 1H); 12.92 (bs, 1H).

MS (m/z) ES+: 415 (MH+).

### 2-Methoxymethyl-8-((styryl)-1H-pyrrolo[2,3-f]isoquinoline-3-carboxylic acid amide

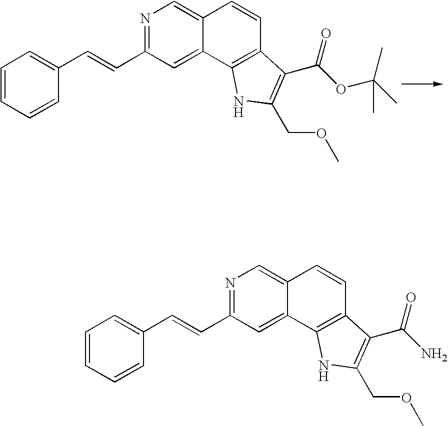

2-Methoxymethyl-8-((E)-styryl)-1H-pyrrolo[2,3-f]isoquinoline-3-carboxylic acid tert-butyl ester (41 mg; 0.099 mmol) is treated with HClconc (1 ml) at room temperature for 2 min., diluted with EtOH and evaporated to dryness. The crystalline residue is suspended in toluene (2 ml), SOCl₂ (2 ml) added and refluxed for 15 min. The reaction mixture is evaporated to dryness, taken up in CH₂Cl₂, cooled in an ice-bath and treated with NH3-gas for 2 min. The reaction mixture is taken up in 2N Na₂CO₃and extracted with EtOAc three times. The combined organic phases are dried over Na₂SO₄, filtered, evaporated to dryness and purified via chromatography (SiO2; TBME/MeOH/NH3conc 98/2/0.5) to yield the title compound as yellowish crystals.

1H-NMR (400 MHz; DMSO-d6): 3.41 (s, 3H); 4.92 (s, 2H); 7.25 (bs, 2H); 7.30-7.47 (m, 4H); 7.71 (bd, 3H); 7.78 (d, 1H); 8.13 (d, 1H); 8.50 (s, 1H); 9.26 (s, 1H); 12.80 (bs, 1H).

MS (m/z) ES+: 358 (MH+).

### EXAMPLE 20

### 2-Methylaminomethyl-8-((E)-styryl)-1H-pyrrolo[2,3-f]isoquinoline-3-carboxylic acid hydrochloride

### 2-[(tert-Butoxycarbonyl-methyl-amino)-methyl]-8-chloro-1H-pyrrolo[2,3-f]isoquinoline-3-carboxylic acid tert-butyl ester

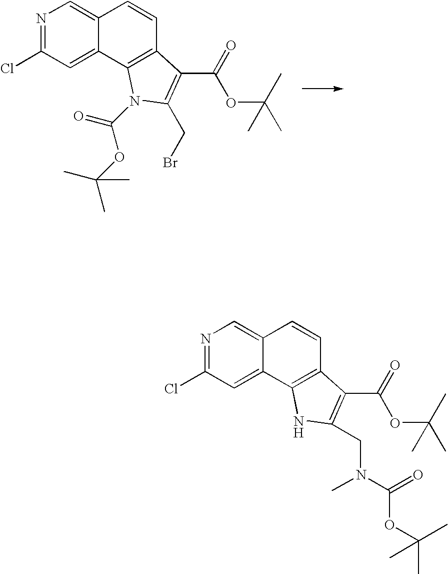

MeNH₂-gas is introduced at room temperature for 2 min. into a solution of 2-bromomethyl-8-chloro-pyrrolo[2,3-f]isoquinoline-1,3-dicarboxylic acid di-tert-butyl ester (350 mg; 0.709 mmol) in dioxane (2 ml). After stirring for 5 min. the reaction mixture is evaporated and purified via chromatography (SiO2; hexanes/acetone 4:1) to yield the title compound as yellowish foam.

1H-NMR (400 MHz; DMSO-d6): 1.37 (s, 9H); 1.62 (s, 9H); 2.93 (bs, 3H); 4.96 (s, 2H); 7.81 (d, 1H); 8.24 (d, 1H); 8.71 (s, 1H); 9.13 (s, 1H); 12.48 (bs, 1H).

MS (m/z) ES+: 446 (MH+).

### 2-[(tert-Butoxycarbonyl-methyl-amino)-methyl]-8-((E)-styryl)-1H-pyrrolo[2,3-f]isoquinoline-3-carboxylic acid tert-butyl ester

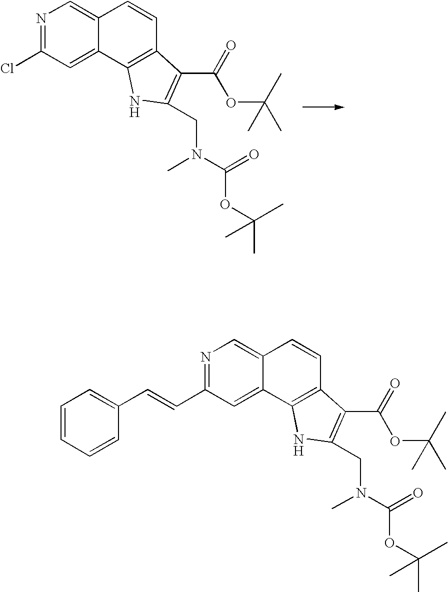

2-[(tert-Butoxycarbonyl-methyl-amino)-methyl]-8-chloro-1H-pyrrolo[2,3-f]isoquinoline-3-carboxylic acid tert-butyl ester (220 mg; 0.493 mmol), trans-phenylvinyl boronic acid (109 mg; 0.74 mmol), K₂CO₃ (85 mg; 0.616 mmol), Pd(dppf)₂Cl₂ (100 mg; 0.123 mmol) are dissolved in DMF (4 ml)/water (1.6 ml) and heated to 90° C. for 1.5 h. The reaction mixture is poured on water and extracted with EtOAc three times. The combined organic phases are dried over Na₂SO₄, filtered, evaporated to dryness and purified via chromatography (SiO2; hexanes/acetone 4/1) to yield the title compound as yellowish crystals.

1H-NMR (400 MHz; DMSO-d6): 1.41 (s, 9H); 1.63 (s, 9H); 2.88 (bs, 3H); 4.98 (s, 2H); 7.09 (d, 1H); 7.30-7.55 (m, 4H); 7.70 (m, 2H); 7.79 (d, 1H); 8.18 (d, 1H); 8.54 (s, 1H); 9.25 (s, 1H); 12.55 (bs, 1H).

MS (m/z) ES+: 514 (MH+).

### 2-Methylaminomethyl-8-((E)-styryl)-1H-pyrrolo[2,3-f]isoquinoline-3-carboxylic acid hydrochloride

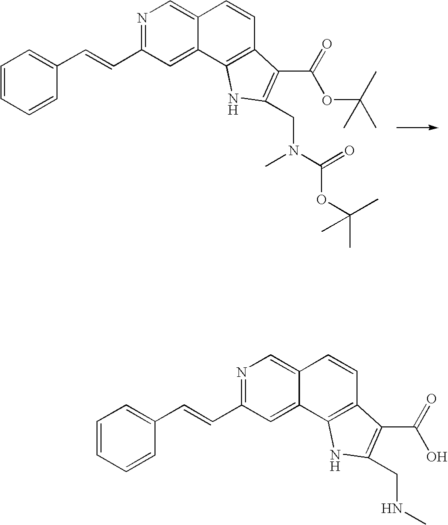

2-[(tert-Butoxycarbonyl-methyl-amino)-methyl]-8-((E)-styryl)-1H-pyrrolo[2,3-f]isoquinoline-3-carboxylic acid tert-butyl ester (215 mg; 0.419 mmol) is treated with HClconc (3 ml) at room temperature for 5 min. The reaction mixture is diluted with toluene several times and repeatedly evaporated to dryness to yield the title compound as yellow crystals.

1H-NMR (400 MHz; DMSO-d6): 2.69 (s, 3H); 4.69 (s, 2H); 7.26-7.51 (m, 5H); 7.70 (d, 2H); 7.89 (d, 1H); 8.00(d, 1H); 8.35 (d, 1H); 8.80 (bs, 1H); 9.55 (s, 1H); 13.19 (bs, 1H); 14.32 (bs, 1H).

MS (m/z) ES+: 358 (MH+).

### EXAMPLE 21

### 8-Methyl-2-((E)-styryl)-9,10-dihydro-8H-3,8,10-triaza-pentaleno[2,1-a]naphthalen-7-one

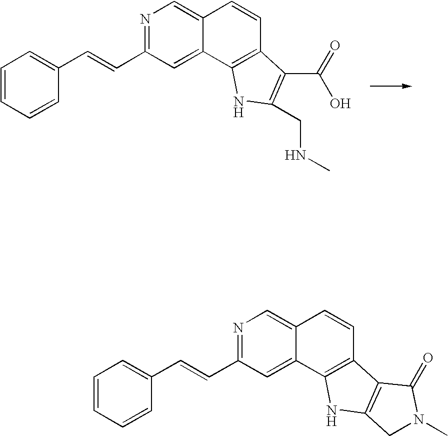

2-Methylaminomethyl-8-((E)-styryl)-1H-pyrrolo[2,3-f]isoquinoline-3-carboxylic acid hydrochloride (40 mg; 0.102 mmol) is suspended in toluene (2 ml) and refluxed with SOCl₂ (1.5 ml) for 20 min. The reaction mixture is evaporated, the solid residue suspended in CH₂Cl₂ (3 ml), cooled in an ice bath and NH₃-gas introduced into the mixture for 2 min. After stirring at room temperature for 5 min. the reaction mixture is poured on 2N Na₂CO₃ and extracted with EtOAc three times. The combined organic phases are dried over Na₂SO₄, filtered, evaporated to dryness and purified via chromatography (SiO2; TBME/MeOH/NH₃conc 95/5/1) to deliver the title compound as brownish crystals.

1H-NMR (400 MHz; DMSO-d6): 3.07 (s, 3H); 4.63 (s, 2H); 7.32 (bt, 1H); 7.30-7.45 (m, 3H); 7.70-7.85 (m, 4H); 7.88 (d, 1H); 8.26 (s, 1H); 9.29 (s, 1H).

MS (m/z) ES+: 340 (MH+).

### EXAMPLE 22

### 2-(4-Hydroxy-phenyl)-9,10-dihydro-8H-3,8,10-triaza-pentaleno[2,1-a]naphthalen-7-one

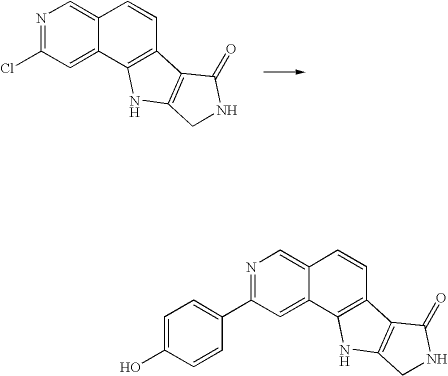

2-Chloro-9,10-dihydro-8H-3,8,10-triaza-pentaleno[2,1-a]naphthalen-7-one (Example 4; 20 mg; 0.078 mmol), 4-hydroxyphenylboronic acid (43 mg; 0.3 mmol), Pd(dppf)₂Cl₂ (32 mg; 0.04 mmol), K₂CO₃ (43 mg; 0.3 mmol) in DMF/water (3 ml/1.2 ml) are heated to 100° C. for 2 h. The reaction mixture is purified via chromatography (SiO₂; TBME/MeOH/NH₃conc 80/20/1.4 to 85/15/1.4) to deliver the title compound as yellow crystals.

1H-NMR (400 MHz; DMSO-d6: 4.55 (s, (2H); 6.94 (d, 2H); 7.75-7.83 (m, 3H); 8.04 (d, 2H); 8.73 (s, 1H); 9.30 (s, 1H); 9.70 (s, 1H); 12.94 (s, 1H).

MS (m/z) ES+: 316 (MH+).

### 2-Chloro-7-oxo-7,9-dihydro-3,8,10-triaza-pentaleno[2,1-a]naphthalene-8,10-dicarboxylic acid di-tert-butyl ester

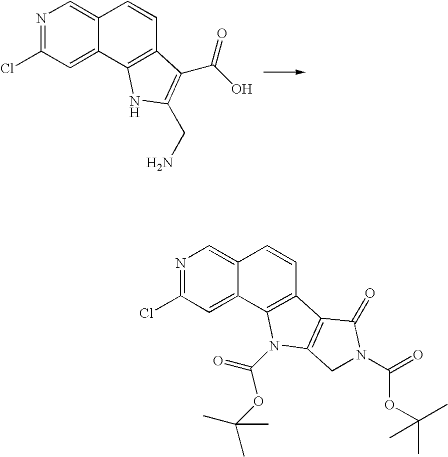

2-Aminomethyl-8-chloro-1H-pyrrolo[2,3-f]isoquinoline-3-carboxylic acid dihydrochloride (Example 3) (170 mg; 0.488 mmol) is suspended in CH₂Cl₂/NEt₃ (12 ml/2 ml) and treated with di-tert-butyl dicarbonate (1.7 g; 7.8 mmol) over night at room temp. The reaction mixture is evaporated and purified via chromatography (SiO2; acetone/hexanes 2/8) to yield the title compound as colorless crystals.

1H-NMR (400 MHz; DMSO-d6): 1.54 (s, 9H); 1.71 (s, 9H); 5.03 (s, 2H); 8.04 (d, 1H); 8.15 (d, 1H); 8.86 (s, 1H); 9.30 (s, 1H).

MS (m/z) ES+: 458 (MH+; 100); 402 (80); 346 (40).

### EXAMPLE 23

### 2-[(E)-2-(4-Methoxy-phenyl)-vinyl]-9,10-dihydro-8H-3,8,10-triaza-pentaleno[2,1-a]naphthalen-7-one

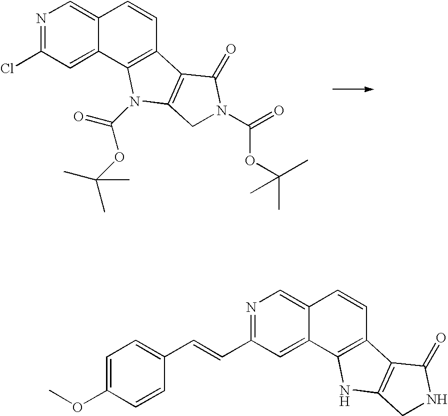

2-[(E)-2-(3-Methoxy-phenyl)-vinyl]-4,4,5,5-tetramethyl-[1,3,2]dioxaborolane (Oronix Nr. 15-7003; 59 mg; 0.27 mmol), 2-chloro-7-oxo-7,9-dihydro-3,8,10-triaza-pentaleno[2,1-a]naphthalene-8,10-dicarboxylic acid di-tert-butyl ester (80 mg; 0.175 mmol), Pd(PPh₃)₂Cl₂ (20 mg), 2N Na₂CO₃ (0.4 ml) are dissolved in 1-propanol (1.3 ml) and treated at 150° C. for 15 min. in the microwave oven. The reaction mixture is poured on water and extracted three times with ethyl acetate. The combined organic phases are dried over Na₂SO₄, filtered, evaporated to dryness and delivered the product as a dark oil, which after purification via chromatography (SiO₂; CH₂Cl₂/MeOH/NH₃conc 95/5/0.5) renders the target compound as yellowish crystals.

1H-NMR (400 MHz; DMSO-d6): 3.82 (s, 3H); 4.55 (s, 2H); 7.02 (d, 2H); 7.30 (d, 1H); 7.67 (d, 2H); 7.75-7.80 (m, 3H); 7.88 (d, 1H); 8.24 (s, 1H); 9.28 (s, 1H); 13.00 (bs, 1H).

MS (m/z) ES+: 356 (MH+).

### EXAMPLE 24

### 2-[(E)-2-(4-Morpholin-4-ylmethyl-phenyl)-vinyl]-9,10-dihydro-8H-3,8,10-triaza-pentaleno[2.1-a]naphthalen-7-one

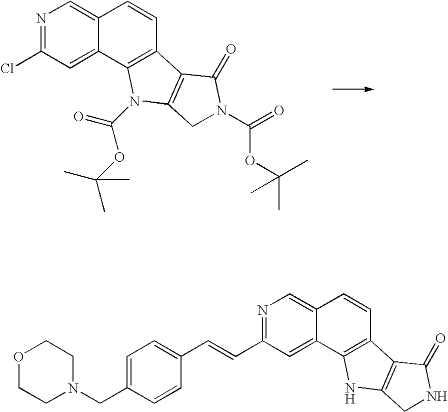

4-{4-[(E)-2-(4,4,5,5-Tetramethyl-[1,3,2]dioxaborolan-2-yl)-vinyl]-benzyl}-morpholine (see example 35) (45 mg; 0.137 mmol), 2-chloro-7-oxo-7,9-dihydro-3,8,10-triaza-pentaleno[2,1-a]naphthalene-8,10-dicarboxylic acid di-tert-butyl ester (52 mg; 0.114 mmol), Pd(PPh3)₂Cl₂ (13 mg), 2N Na₂CO₃ (0.3 ml) are dissolved in 1-propanol (0.9 ml) and treated at 150° C. for 15 min. in the microwave oven. The reaction mixture is poured on water and extracted three times with ethyl acetate. The combined organic phases are dried over Na₂SO₄, filtered, evaporated to dryness and delivered the product as a dark oil, which after purification via chromatography (SiO₂; CH₂Cl₂/MeOH/NH₃conc 90/10/1) renders the target compound as yellow solid.

1H-NMR (400 MHz; DMSO-d6): 2.40 (m, 4H); 3.51 (s, 2H); 3.61 (m, 4H); 4.56 (s, 2H); 7.38-7.42 (m, 3H); 7.68 (d, 2H); 7.75-7.82 (m, 3H); 7.88 (d, 1H); 8.29 (s, 1H); 9.30 (s, 1H); 13.01 (bs, 1H).

MS (m/z) ES+: 425 (MH+).

### EXAMPLE 25

### 2-{(E)-2-[3-(2-Morpholin-4-yl-ethyl)-phenyl]-vinyl}-9,10-dihydro-8H-3,8,10-triaza-pentaleno[2,1-a]naphthalen-7-one

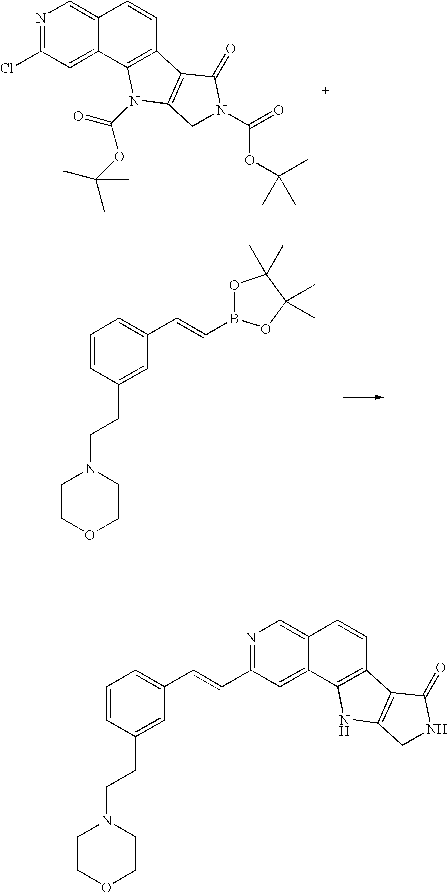

4-(2-{3-[(E)-2-(4,4,5,5-Tetramethyl-[1,3,2]dioxaborolan-2-yl)-vinyl]phenyl}ethyl)-morpholine (WO 2004058762) (52 mg; 0.151 mmol),), 2-chloro-7-oxo-7,9-dihydro-3,8,10-triaza-pentaleno[2,1-a]naphthalene-8,10-dicarboxylic acid di-tert-butyl ester (53 mg; 0.116 mmol), Pd(PPh3)₂Cl₂ (13 mg), 2N Na₂CO₃ (0.25 ml) are dissolved in 1-propanol (0.8ml) and treated at 150° C. for 15 min. in the microwave oven. The reaction mixture is poured on water and extracted three times with ethyl acetate. The combined organic phases are dried over Na₂SO₄, filtered, evaporated to dryness and delivered the product as a dark oil, which after purification via chromatography (SiO₂; CH₂Cl₂/MeOH/NH₃conc 95/5/0.5) renders the target compound as yellow solid.

1H-NMR (400 MHz; DMSO-d6): 2.50 (m, 4H); 2.67 (m, 2H); 2.80 (m, 2H); 3.60 (m, 4H); 4.50 (s, 2H); 7.20 (bd, 1H); 7.30 (d, 1H); 7.40 (d, 1H); 7.50 (d, 1H); 7.60 (s, 1H); 7.75-7.84 (m, 3H); 7.90 (d, 1H); 8.30 (s, 1H); 9.30 (s, 1H); 13.0 (s, 1H).

MS (m/z) ES+: 439 (MH+).

### EXAMPLE 26

### 8-Benzyl-2-((E)-styryl)-9,10-dihydro-8H-3,8,10-triaza-pentaleno[2,1-a]naphthalen-7-one

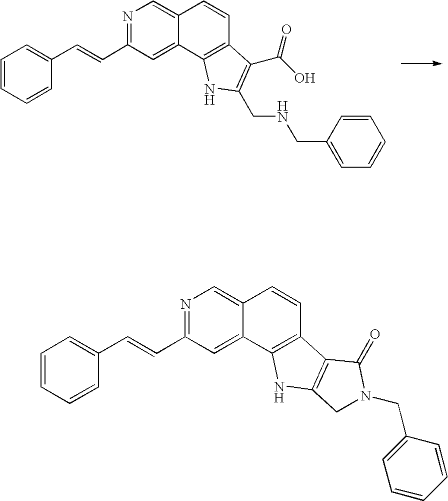

2-(Benzylamino-methyl)-8-((E)-styryl)-1H-pyrrolo[2,3-f]isoquinoline-3-carboxylic acid (prepared in analogy to Example 25) (80 mg; 1.58 mmol) is refluxed in toluene/SOCl₂ (2 ml/0.5 ml) for 5 minutes. The reaction mixture is evaporated, dissolved in CH₂Cl₂/NEt₃ (6 ml/0.4 ml), left at room temp. for 10 minutes, evaporated and purified via chromatography (SiO2; acetone/hexanes 3/7 to 4/6) to yield the title compound as yellow crystals.

1H-NMR (400 MHz; DMSO-d6): 4.54 (s, 2H); 4.71 (s, 2H); 7.25-7.45 (m, 9H); 7.70 (m, 3H); 7.79 (d, 1H); 7.90 (d, 1H); 8.29 (s, 1H); 9.29 (s, 1H); 13.04 (bs, 1H).

MS (m/z) ES+: 416 (MH+).

### EXAMPLE 27

### 2-(3-Methoxy-phenyl)-9,10-dihydro-8H-3,8,10-triaza-pentaleno[2,1-a]naphthalen-7-one

### 2-(3-Methoxy-phenyl)-7-oxo-7,9-dihydro-3,8,10-triaza-pentaleno[2,1-a]naphthalene-8,10-dicarboxylic acid di-tert-butyl ester

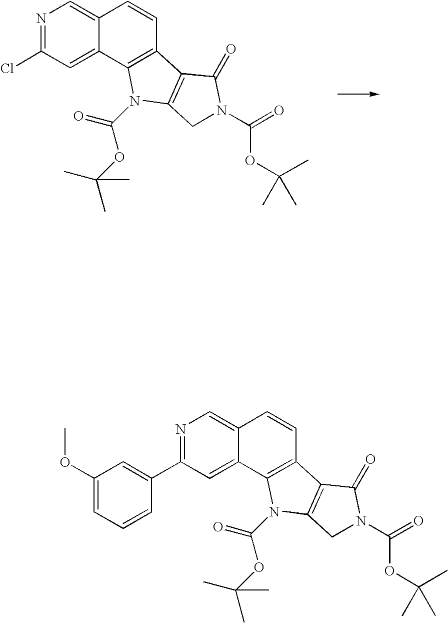

2-Chloro-7-oxo-7,9-dihydro-3,8,10-triaza-pentaleno[2,1-a]naphthalene-8,10-dicarboxylic acid di-tert-butyl ester (30 mg; 0.0.06 mmol), 3-methoxyphenylboronic acid (40 mg; 0.26 mmol), Pd(dppf)₂Cl₂ (27 mg; 0.03 mmol), K₂CO₃ (36 mg; 0.26 mmol) in DMF/water (3 ml/1.2 ml) are heated at 100° C. for 4 h. Di-tert-butyl dicarbonate (200 mg; 1 mmol) and DMAP (4 mg) dissolved in 1,4-dioxane (4 ml) is added and the reaction mixture refluxed for 5 minutes. The reaction mixture is evaporated and purified via chromatography (SiO₂; EtOAc/hexanes 3/7) to yield the title compound as yellow crystals.

1H-NMR (400 MHz; DMSO-d6): 1.55 (s, 9H); 1.71 (s, 9H); 3.87 (s, 3H); 5.05 (s, 2H); 7.03 (bd, 1H); 7.47 (t, 1H); 7.76 (bs, 2H); 8.01 (d, 1H); 8.13 (d, 1H); 9.25 (s, 1H); 9.48 (s, 1H).

MS (m/z) ES+: +). 530 (MH+).

### 2-(3-Methoxy-phenyl)-9,10-dihydro-8H-3,8,10-triaza-pentaleno[2,1-a]naphthalen-7-one

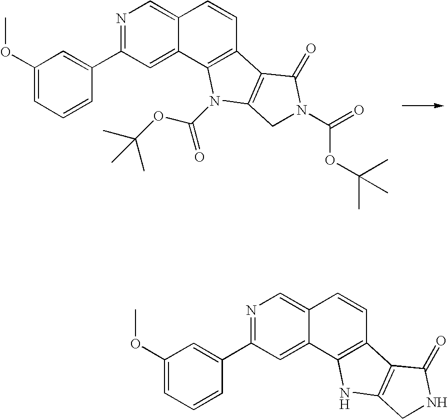

2-(3-Methoxy-phenyl)-7-oxo-7,9-dihydro-3,8,10-triaza-pentaleno[2,1-a]naphthalene-8,10-dicarboxylic acid di-tert-butyl ester (10 mg; 0.019 mmol) is treated with TFA (1 ml) for 45 minutes at room temp. TFA is evaporated and the residue taken up in water, alkalized with NH₃conc. The resulting solid is filtered and washed with TBME to yield the title compound as yellowish crystals.

1H-NMR (400 MHz; DMSO-d6): 3.89 (s, 3H); 4.57 (s, 2H); 7.05 (dd, 1H); 7.48 (dd, 1H); 7.79-7.83 (m, 4H); 7.91 (d, 1H); 8.91 (s, 1H); 9.39 (s, 1H); 13.02 (bs, 1H).

MS (m/z) ES+: 330 (MH+).

### EXAMPLE 28

### 2-[3-(3-Methoxy-propoxy)-phenyl]-9,10-dihydro-8H-3,8,10-triaza-pentaleno[2,1-a]naphthalen-7-one

### 2-[3-(3-Methoxy-propoxy)-phenyl]-7-oxo-9,10-dihydro-8H-3,8,10-triaza-pentaleno[2,1-a]naphthalen-8,10-dicarboxylic acid di-tert-butyl ester

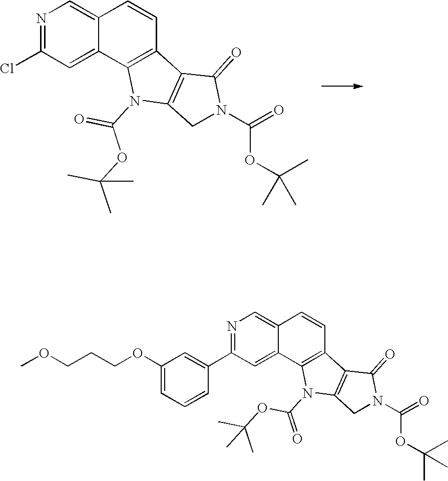

2-Chloro-7-oxo-7,9-dihydro-3,8,10-triaza-pentaleno[2,1-a]naphthalene-8,10-dicarboxylic acid di-tert-butyl ester (30 mg; 0.06 mmol) and 3-(methoxypropoxy)-phenylboronic acid (WO 2005077932; 55 mg; 0.26 mmol) are treated in analogy to example 27 to yield the title compound as colorless crystals.

1H-NMR (400 MHz; DMSO-d6): 1.55 (s, 9H); 1.71 (s, 9H); 2.02 (m, 2H); 3.27 (s, 3H); 3.52 (t, 2H); 4.14 (t, 2H); 5.04 (s, 2H); 7.02 (bd, 1H); 7.45 (t, 1H); 7.75 (bs, 2H); 8.00 (d, 1H); 8.12 (d, 1H); 9.24 (s, 1H); 9.47 (s, 1H).

MS (m/z) ES+: 588 (MH+).

### 2-[3-(3-Methoxy-propoxy)-phenyl]-9,10-dihydro-8H-3,8,10-triaza-pentaleno[2,1-a]naphthalen-7-one

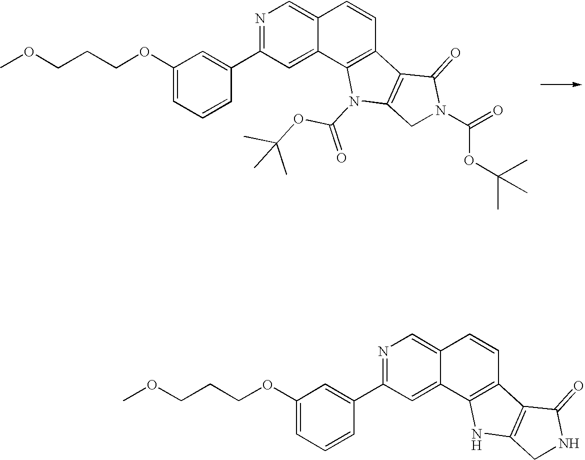

2-[3-(3-Methoxy-propoxy)-phenyl]-7-oxo-9,10-dihydro-8H-3,8,10-triaza-pentaleno[2,1-a]naphthalen-8,10-dicarboxylic acid di-tert-butyl ester (19 mg; 0.03 mmol) is treated with HClconc. (1 ml) for 5 min. The reaction mixture is evaporated and the residue taken up in MeOH/NH₃conc. The mixture is evaporated again delivering a solid, from which the title compound is extracted with MeOH. The desired compound is obtained as yellow crystals.

1H-NMR (400 MHz; DMSO-d6): 2.04 (m, 2H); 3.28 (s, 3H); 3.54 (t, 2H); 4.17 (t, 2H); 4.61 (s, 2H); 7.13 (bd, 1H); 7.53 (t, 1H); 7.75 (bs, 2H); 7.93 (bd, 2H); 8.04 (bd, 1H); 9.08 (s, 1H); 9.56 (s, 1H); 13.34 (bs, 1H).

MS (m/z) ES+: 388 (MH+).

### EXAMPLE 29

### 2-Pyridin-3-yl-9,10-dihydro-8H-3,8,10-triaza-pentaleno[2,1-a]naphthalen-7-one

### 2-Pyridin-3-yl-7-oxo-9,10-dihydro-8H-3,8,10-triaza-pentaleno[2,1-a]naphthalene-8-carboxylic acid tert-butyl ester

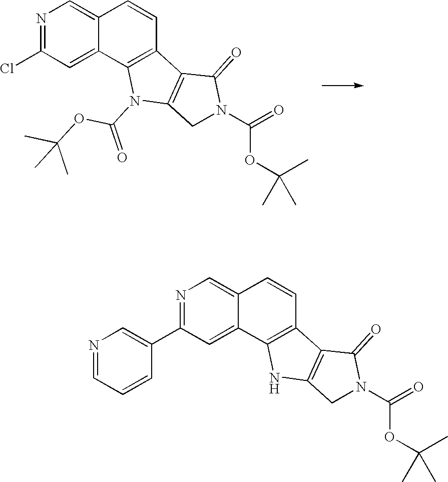

2-Chloro-7-oxo-7,9-dihydro-3,8,10-triaza-pentaleno[2,1-a]naphthalene-8,10-dicarboxylic acid di-tert-butyl ester (30 mg; 0.06 mmol), Pd(PPh₃)₂Cl₂ (23 mg; 0.032 mmol), 3-(1,1,1-tributylstannyl)pyridine (145 mg; 0.39 mmol) in DMF/xylene (0.5 ml/0.5 ml) are heated to 140° C. for 1 h. The reaction mixture is evaporated and purified via chromatography (SiO₂; acetone/hexanes 3/7 to 6/4) to yield the title compound as yellow solid.

1H-NMR (400 MHz; DMSO-d6): 1.55 (s, 9H); 5.02 (s, 2H); 7.61 (m, 1H); 7.91 (d, 1H); 7.96 (d, 1H); 8.51 (bd, 1H); 8.64 (bd, 1H); 8.99 (s, 1H); 9.38 (s, 1H); 9.46 (s, 1H); 13.23 (s, 1H).

MS (m/z) ES+: 400 (MH+).

### 2-Pyridin-3-yl-9,10-dihydro-8H-3,8,10-triaza-pentaleno[2,1-a]naphthalen-7-one

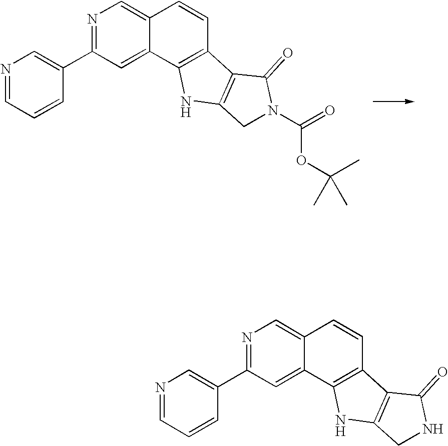

2-Pyridin-3-yl-7-oxo-9,10-dihydro-8H-3,8,10-triaza-pentaleno[2,1-a]naphthalene-8-carboxylic acid tert-butyl ester (13 mg; 0.03 mmol) is treated with HClconc (1 ml) at room temp. for 5 minutes and then evaporated. The solid is taken up in water (0.5 ml) alkalized with NH₃conc and evaporated again. The resulting solid is washed with water (2×1 ml) followed by CH₂Cl₂ (1 ml) which delivers the target compound as yellow solid.

1H-NMR (400 MHz; DMSO-d6): 4.55 (s, 2H); 7.30 (bs, 1H); 7.52-7.58 (m, 2H); 7.81 (d, 1H); 7.97 (d, 1H); 8.55 (bd, 1H); 8.64 (bd, 1H); 8.90 (s, 1H); 9.41 (s, 1H); 12.66 (bs, 1H).

MS (m/z) ES+: 301 (MH+).

### EXAMPLE 30

### 2-(4-Methoxy-phenyl)-9,10-dihydro-8H-3,8,10-triaza-pentaleno[2,1-a]naphthalen-7-one

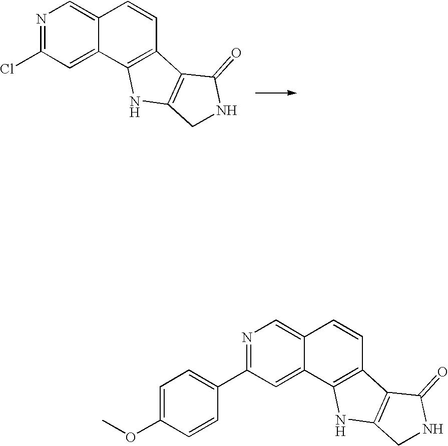

2-Chloro-9,10-dihydro-8H-3,8,10-triaza-pentaleno[2,1-a]naphthalen-7-one (Example 4; 20 mg; 0.078 mmol), 4-methoxyphenylboronic acid (24 mg; 0.15 mmol), Pd(dppf)₂Cl₂ (32 mg; 0.04 mmol), K₂CO₃ (32 mg; 0.22 mmol) in DMF/water (2 ml/0.8 ml) are heated to 100° C. for 3.5 h. The reaction mixture is purified via chromatography (SiO₂; TBME/MeOH/NH₃conc 90/10/1.4 to 85/15/1.4) to deliver the title compound as yellow crystals.

1H-NMR (400 MHz; DMSO-d6): 3.86 (s, 3H); 4.58 (s, 2H); 7.17 (d, 2H); 7.85 (m, 2H); 7.91 (d, 1H); 8.13 (d, 2H); 8.87 (s, 1H); 9.41 (s, 1H); 13.12 (bs, 1H).

MS (m/z) ES+: 330 (MH+)

### EXAMPLE 31

### 2-(2-Fluoro-phenyl)-9,10-dihydro-8H-3,8,10-triaza-pentaleno[2,1-a]naphthalen-7-one

### 2-(2-Fluoro-phenyl)-7-oxo-9,10-dihydro-8H-3,8,10-triaza-pentaleno[2,1-a]naphthalen-8-carboxylic acid tert-butyl ester

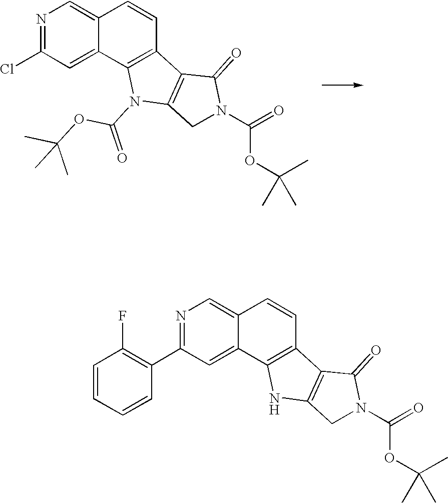

2-Chloro-7-oxo-7,9-dihydro-3,8,10-triaza-pentaleno[2,1-a]naphthalene-8,10-dicarboxylic acid di-tert-butyl ester (30 mg; 0.06 mmol), Pd(PPh₃)₂Cl₂ (27 mg; 0.033 mmol), PPh₃ (17 mg; 0.06 mmol), Cs₂CO₃ (86 mg; 0.26 mmol), 2-fluorophenylboronic acid (37 mg; 0.26 mmol) are dissolved in DMF/water (3 ml/1.2 ml) and heated at 120° C. in the microwave oven. The reaction mixture is evaporated and purified via chromatography (SiO₂; EtOAc/hexanes 4/6 to 1/1) to yield the title compound as colorless crystals.

1H-NMR (400 MHz; DMSO-d6): 1.55 (s, 9H); 4.98 (s, 2H); 7.35-7.45 (m, 2H); 7.50 (m, 1H); 7.90 (d, 1H); 7.97 (d, 1H); 8.13 (dt, 1H); 8.76 (s, 1H); 9.45 (s, 1H); 13.20 (bs, 1H).

MS (m/z) ES+: 418 (MH+).

### 2-(2-Fluoro-phenyl)-9,10-dihydro-8H-3,8,10-triaza-pentaleno[2,1-a]naphthalen-7-one

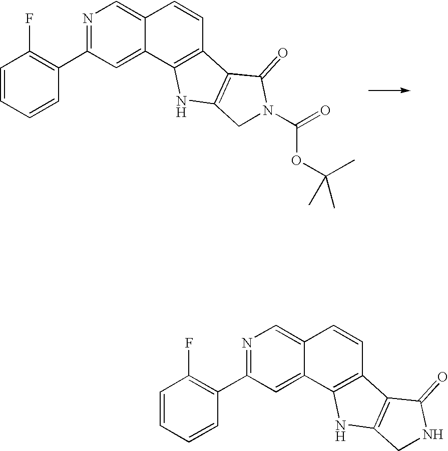

2-(2-Fluoro-phenyl)-7-oxo-9,10-dihydro-8H-3,8,10-triaza-pentaleno[2,1-a]naphthalen-8-carboxylic acid tert-butyl ester (7 mg; 0.076 mmol) is dissolved in HClconc (1 ml) and stirred for 10 min at room temp. The resulting suspension is evaporated, taken up in MeOH, alkalized with NH₃conc and evaporated. The resulting solid is triturated with water and dried to deliver the title compound as yellow crystals.

1H-NMR (400 MHz; DMSO-d6): 4.57 (s, 2H); 7.35-7.45 (m, 2H); 7.48-7.53 (m, 1H); 7.81 (m, 2H); 7.94 (d, 1H); 8.11 (t, 1H); 8.73 (s, 1H); 9.42 (s, 1H); 13.12 (bs, 1H).

MS (m/z) ES+: 318 (MH+).

### EXAMPLE 32

### 2-(3-Methanesulfonyl-phenyl)-9,10-dihydro-8H-3,8,10-triaza-pentaleno[2,1-a]naphthalen-7-one

### 2-(3-Methanesulfonyl-phenyl)-7-oxo-9,10-dihydro-8H-3,8,10-triaza-pentaleno[2,1-a]naphthalen-8,10-dicarboxylic acid di-tert-butyl ester

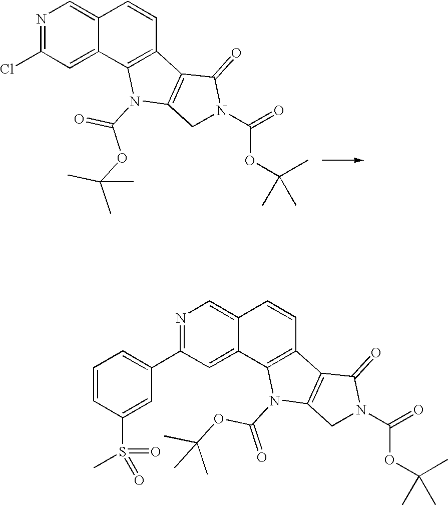

2-Chloro-7-oxo-7,9-dihydro-3,8,10-triaza-pentaleno[2,1-a]naphthalene-8,10-dicarboxylic acid di-tert-butyl ester (30 mg; 0.06 mmol), Pd(PPh₃)₂Cl₂ (27 mg; 0.033 mmol), PPh₃ (17 mg; 0.06 mmol), Cs₂CO₃ (86 mg; 0.26 mmol), 3-(methylsulfonyl)phenylboronic acid (52 mg; 0.26 mmol) and 2-dicyclohexylphosphino-2′-(N,N-dimethylamino)biphenyl (4 mg) are dissolved in DMF/water (3 ml/1.2 ml) and heated at 120° C. in the microwave oven for 45 minutes. The reaction mixture is combined with di-tert-butyl dicarbonate (200 mg; 0.4 mmol) and DMAP (4 mg) in 1,4-dioxane and refluxed for 5 minutes. The reaction mixture is evaporated and purified via chromatography (SiO₂; EtOAc/hexanes 4/6 to 1/1) to yield the title compound as colorless crystals.

1H-NMR (400 MHz; DMSO-d6): 1.55 (s, 9H); 1.71 (s, 9H); 3.26 (s, 3H); 5.06 (s, 2H); 7.85 (t, 1H); 7.99 (d, 1H); 8.05 (d, 1H); 8.17 (d, 1H); 8.48 (d, 1H); 8.74 (s, 1H); 9.34 (s, 1H); 9.54 (s, 1H).

MS (m/z) ES+: 578 (MH+).

### 2-(3-Methanesulfonyl-phenyl)-9,10-dihydro-8H-3,8,10-triaza-pentaleno[2,1-a]naphthalen-7-one

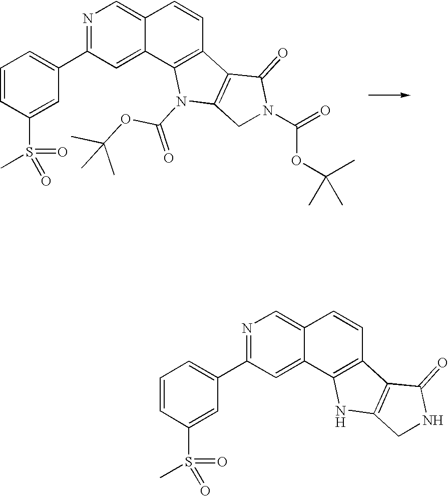

2-(3-Methanesulfonyl-phenyl-7-oxo-9,10-dihydro-8H-3,8,10-triaza-pentaleno[2,1-a]naphthalen-8,10-dicarboxylic acid di-tert-butyl ester (22 mg; 0.04 mmol) is treated with HClconc. for 10 min at room temp. The reaction mixture is evaporated, taken up in MeOH/NH₃conc. and evaporated again. The resulting solid is washed with water and subsequently with CH₂Cl₂ to deliver the title compound as colorless crystals.

1H-NMR (400 MHz; DMSO-d6): 3.31 (s, 3H); 4.58 (s, 2H); 7.83-7.87 (m, 3H); 9.95 (d, 1H); 8.03 (d, 1H); 8.56 (d, 1H); 8.75 (s, 1H); 9.04 (s, 1H); 9.43 (s, 1H); 13.13 (s, 1H).

MS (m/z) ES+: 378 (MH+).

### EXAMPLE 33

### 2-(2-Trifluoromethyl-phenyl)-9,10-dihydro-8H-3,8,10-triaza-pentaleno[2,1-a]naphthalen-7-one

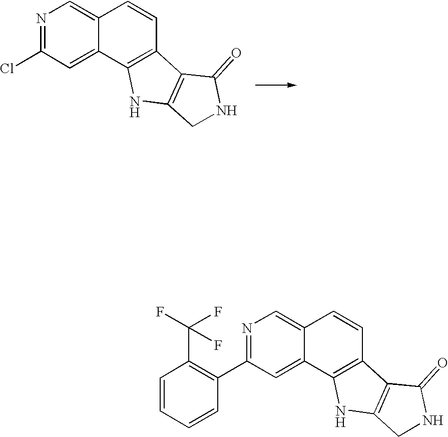

2-Chloro-9,10-dihydro-8H-3,8,10-triaza-pentaleno[2,1-a]naphthalen-7-one (Example 4; 20 mg; 0.078 mmol), 2-(trifluoromethyl)phenylboronic acid (59 mg; 0.3 mmol), PPh₃ (20 mg; 0.078 mmol), Pd(dppf)₂Cl₂ (32 mg; 0.04 mmol), K₂CO₃ (43 mg; 0.3 mmol) in DMF/water (3 ml/1.2 ml) are heated to 100° C. for 2.5 h. The reaction mixture is purified via chromatography (SiO₂; CH₂Cl₂/MeOH 94/6) to deliver the title compound as yellow crystals.

1H-NMR (400 MHz; DMSO-d6: 4.55 (s, 2H); 7.65-8.10 (m, 7H); 8.36 (s, 1H); 9.38 (s, 1H); 13.00 (s, 1H).

MS (m/z) ES+: 368 (MH+).

### EXAMPLE 34

### 2-(3-Fluoro-phenylamino)-9,10-dihydro-8H-3,8,10-triaza-pentaleno[2,1-a]naphthalen-7-one

### 2-(3-Fluoro-phenylamino)-7-oxo-7,9-dihydro-3,8,10-triaza-pentaleno[2,1-a]naphthalene-8-carboxylic acid tert-butyl ester

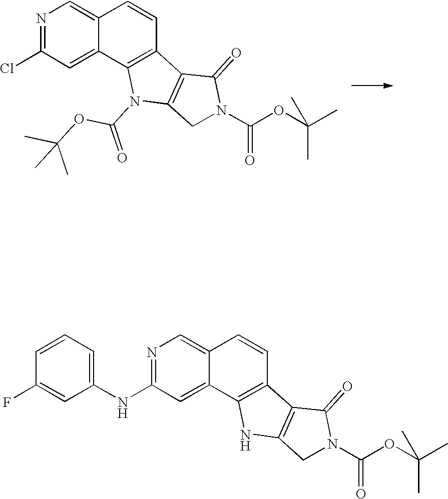

2-Chloro-9,10-dihydro-8H-3,8,10-triaza-pentaleno[2,1-a]naphthalen-7-one (Example 4; 30 mg; 0.066 mmol), 3-fluoroaniline (600 mg; 5.4 mmol), R(+)-BINAP (7.5 mg; 0.012 mmol), Cs₂CO₃ (85 mg; 0.26 mmol), Pd(dppf)₂Cl₂ (26 mg; 0.03 mmol), PPh₃ (18 mg; 0.07 mmol), 2-dicyclohexylphosphino-2′-(N,N-dimethylamino)biphenyl (4 mg) are dissolved in DMF (7 ml) and heated at 160° C. in the microwave oven for 1 h. The reaction mixture is purified via chromatography (SiO₂; EtOAc/hexanes (1/1) to yield the title compound as brownish solid.

1H-NMR (400 MHz; DMSO-d6: 1.57 (s, 9H); 4.96 (s, 2H); 6.72 (m, 1H); 7.32 (m, 2H); 7.51 (bd, 1H); 7.60-7.74 (m, 3H); 9.08 (s, 1H); 9.37 (s, 1H); 13.15 (bs, 1H).

MS (m/z) ES−: 431 (MH−).

### 2-(3-Fluoro-phenylamino)-9,10-dihydro-8H-3,8,10-triaza-pentaleno[2,1-a]naphthalen-7-one

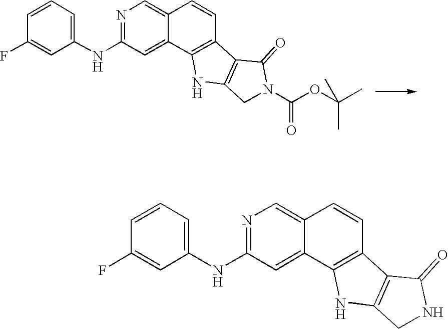

2-(3-Fluoro-phenylamino)-7-oxo-7,9-dihydro-3,8,10-triaza-pentaleno[2,1-a]naphthalene-8-carboxylic acid tert-butyl ester (88 mg; 0.2 mmol) are suspended in HClconc (2 ml) and stirred for 10 minutes. The reaction mixture is evaporated and taken up in MeOH/NH₃conc (2 ml/1 ml) and evaporated again. The resulting solid is washed with water and subsequently with CH₂Cl₂ and delivers the title compound as slightly colored solid.

1H-NMR (400 MHz; DMSO-d6: 4.52 (s, 2H); 6.70 (m, 1H); 7.33 (m, 2H); 7.51 (bd, 1H); 7.66 (s, 2H); 7.70 (s; 1H); 7.76 (s, 1H); 9.04 (s, 1H); 9.32 (s, 1H); 12.77 (bs, 1H).

MS (m/z) ES+: 333 (MH+).

### EXAMPLE 35

### 2-[(E)-2-(4-Morpholin-4-ylmethyl-phenyl)-vinyl]-9,10,11,12-tetrahydro-8H-3,8,12-triaza-naphtho[2,1-a]azulen-7-one

### 4-(4-Ethynyl-benzyl)-morpholine

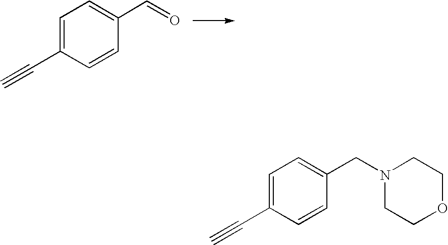

4-Ethynylbenzaldehyde (1.30 g, 10.0 mmol) are dissolved in 50 ml methanol/acetic acid (93/7), then 0.96 g (11.0 mmol) morpholine followed by 0.80 g (10.0 mmol) sodium cyanoborohydride are added. This mixture is stirred at room temperature for 20 hours. Afterwards 5 ml 2N hydrochloric acid is added and 20 minutes stirred at room temperature. The solution is alkalized with 40% NaOH and the title compound extracted with ethyl acetate. The crude product is purified by chromatography on silica gel (ethyl acetate/methanol/ammonia:9/1/0.1).

¹H-NMR (400 MHZ, DMSO-d6): 2.32 (t, 4H), 3.45 (s, 2H), 3.55 (t, 4H), 4.12 (s, 1H), 7.29 (d, 2H), 7.40 (d, 2H).

MS (ESI⁺) m/z: 202 [M]⁺

### 4-{4-[(E)-2-(4,4,5,5-Tetramethyl-[1,3,2]dioxaborolan-2-yl)-vinyl]-benzyl}-morpholine

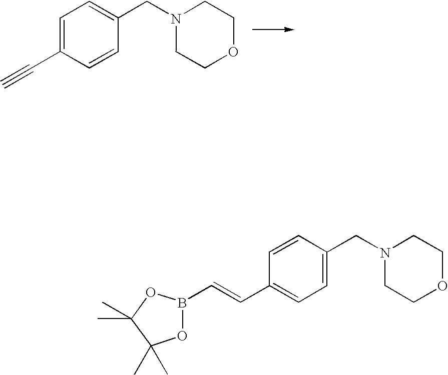

4-(4-Ethynyl-benzyl)-morpholine (3.0 g, 14.9 mmol) is dissolved in 100 ml dichloromethane and 5.85 g, (45.7 mmol) 4,4,5,5-tetramethyl-[1,3,2]dioxaborolane is added. The solution is degassed by introduction of a stream of argon, Rh(PPh₃)₃Cl (140 mg, 0.15 mmol) is added and the mixture stirred at room temperature for 24 hours. The reaction is quenched with saturated ammonium chloride solution, extracted into ethyl acetate, the organic phase is dried over Na₂SO₄ and the solvent evaporated. The crude product is purified by chromatography on silica gel (cyclohexan/ethyl acetate 3/1).

¹H-NMR (400 MHZ, DMSO-d6): 1.24 (s, 12H), 2.30-2.35 (m, 4H), 3.45 (s, 2H), 3.52-5.59 (m, 4H), 6.09 (d, 1H), 7.20-7.30 (m, 3H), 7.50 (d, 2H).

MS (ESI⁺) m/z: 330 [MH]⁺

### 2-[(E)-2-(4-Morpholin-4-ylmethyl-phenyl)-vinyl]-9,10,11,12-tetrahydro-8H-3,8,12-triaza-naphtho[2,1-a]azulen-7-one

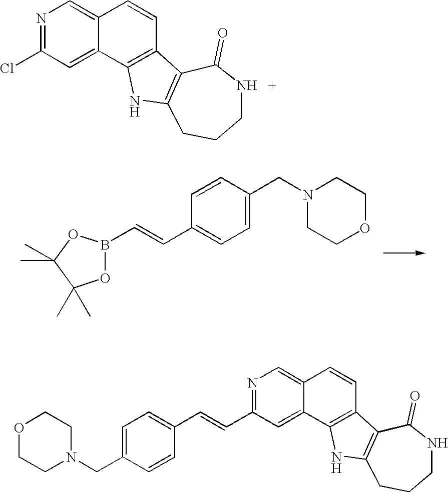

4-{4-[(E)-2-(4,4,5,5-Tetramethyl-[1,3,2]dioxaborolan-2-yl)-vinyl]benzyl}-morpholine (59.9 mg, 0.18 mmol) and 2-chloro-9,10,11,12-tetrahydro-8H-3,8,12-triaza-naphtho[2,1-a]azulen-7-one (40.0 mg, 0.14 mmol) are dissolved in 1 ml n-propanol and 0.3 ml 2N sodiumcarbonate solution. The solution is degassed by introduction of a stream of argon, Pd(PPh₃)₂Cl₂ (10 mg, 0.015 mmol) is added and the mixture is heated to 150° C. for 15 minutes in a microwave oven. After evaporation of the solvents the crude product is purified by chromatography on silica gel (ethyl acetate/methanol/ammonia:95/5/0.5 to 85/15/1.5).

¹H-NMR (400 MHZ, DMSO-d6): 2.00-2.10 (m, 2H), 2.30-2.41 (m, 4H), 3.20-3.31. (m, 4H), 3.49 (s, 2H), 3.52-3.62 (m, 4H), 7.34-7.40 (m, 3H), 7.52-7.68 (m, 3H), 7.76 (d, 1H), 8.23 (s, 1H), 8.44 (d, 1H), 9.21 (s, 1H), 12.51 (brs, 1NH).

MS (ESI⁺) m/z: 453 [MH]⁺

### EXAMPLE 36

### 2-Pyridin-3-yl-9,10,11,12-tetrahydro-8H-3,8,12-triaza-naphtho[2,1-a]azulen-7-one

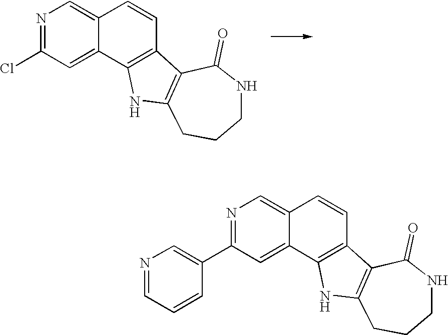

2-Chloro-9,10,11,12-tetrahydro-8H-3,8,12-triaza-naphtho[2,1-a]azulen-7-one (156 mg; 0.54 mmol), Pd(PPh₃)₂Cl₂ (192 mg; 0.27 mmol), 3-(1,1,1-tributylstannyl)pyridine (1.2 g; 3.2 mmol) in DMF/xylene (3 ml/3 ml) are heated to 130° C. for 5 h. The reaction mixture is evaporated and purified via chromatography (SiO₂; acetone/hexanes 8/2 to 1/0) to yield a mixture of title compound and starting material (˜6:1). The title compound is separated from the starting material by acidic extraction with 2N HCl/1-BuOH. After alkalization of the acidic aq. phase with Na₂CO₃, the aqueous phase is extracted with 1-BuOH three times. The combined organic phases are dried over Na₂SO₄, filtered and evaporated. The solid residue is washed with water, CH₂Cl₂ and TBME to yield the title compound as yellow crystals.

1H-NMR (400 MHz; DMSO-d6: 2.12 (m, 2H); 3.30 (m, 4H); 7.60 (m, 2H); 7.73 (d, 1H); 8.52 (d, 2H); 8.65 (d, 1H); 8.93 (s, 1H); 9.37 (s, 1H); 9.39 (s, 1H); 12.53 (bs, 1H).

MS (m/z) ES+: 329 (MH+).

### 3-(3-Chloro-isoquinolin-5-ylamino)-cyclopent-2-enone

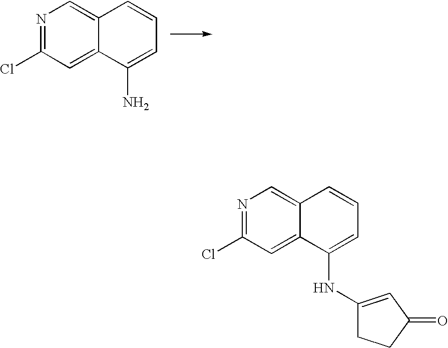

5-Aminoisoquinoline (4 g; 22.47 mmol) and 1,3-cyclopentanedione (3 g; 30.6 mmol) are dissolved in MeOH (100 ml) and evaporated to dryness. The residue is heated as a melt to 120° C. for 20 minutes. The crystalline residue is used for the next step without purification.

### 2-Chloro-9,10-dihydro-8H-cyclopenta[4,5]pyrrolo[2,3-f]isoquinolin-7-one

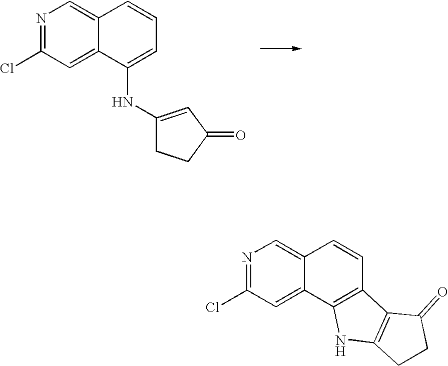

3-(3-Chloro-isoquinolin-5-ylamino)-cyclopent-2-enone (6.1 g; 23.6 mmol), Pd(OAc)₂ (1.4 g; 6.5 mmol), Cu(OAc)₂ (14.4 g; 72.3 mmol) in DMF (300 ml) are heated to 120° C. for 45 minutes. The reaction mixture is filtered, diluted with acetone (2.5 l) and passed through a short column of SiO2. Evaporation delivered a brown-green residue, which is suspended in CH₂Cl₂/MeOH (9:1) and washed with 2N HCl several times. The brown solid is washed with water several times and dried which yields the target compound.

1H-NMR (400 MHz; DMSO-d6): 2.50 (s, 2H); 3.22 (s, 2H); 7.85 (d, 1H); 7.93 (d, 1H); 8.33 (s, 1H); 9.18 (s, 1H); 13.07 (s, 1H).

MS (m/z) ES+: 258 (MH+)

### 2-Chloro-9,10-dihydro-8H-cyclopenta[4,5]pyrrolo[2,3-f]isoquinolin-7-one oxime

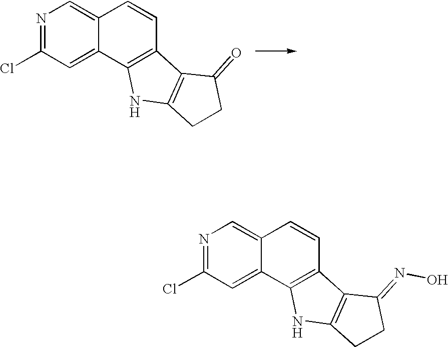

2-Chloro-9,10-dihydro-8H-cyclopenta[4,5]pyrrolo[2,3-f]isoquinolin-7-one (2.2 g; 8.6 mmol) H₂NOH. HCl 2.2 g; 31.4 mmol) and pyridine (2.2 g; 27.8 mmol) are dissolved in ethanol (300 ml) and refluxed for 3 hours. The reaction mixture is filtered and evaporated to a volume of 50 ml. The target compound crystallizes as off-white solid.

1H-NMR (400 MHz; DMSO-d6): 3.26 (bs, 4H); 7.79 (d, 1H); 8.24 (d, 1H); 8.37 (s, 1H); 9.15 (s, 1H); 11.12 (bs, 1H); 13.20 (bs, 1H).

MS (m/z) ES+: 273 (MH+)

### EXAMPLE 37

### 2-Chloro-8,9,10,11-tetrahydro-3,8,11-triaza-benzo[a]fluoren-7-one

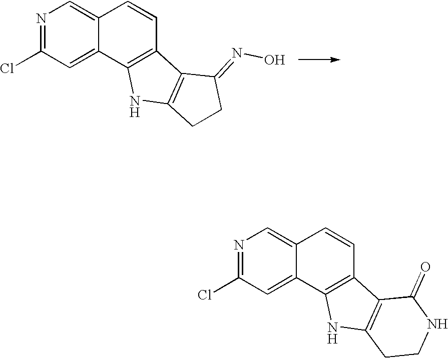

2-Chloro-9,10-dihydro-8H-cyclopenta[4,5]pyrrolo[2,3-f]isoquinolin-7-one oxime (1.56 g; 5.75 mmol) in polyphosphoric acid (60 g) is heated to 130° C. for 75 minutes. The reaction mixture is cooled, combined with ice (200 g) and poured on water (200 ml) containing Na₂CO₃ (78 g). The fine suspension is filtered and dissolved in methanol (˜300 ml) and filtered again. The organic phase is evaporated and deliver the title compound as brownish crystals

1H-NMR (400 MHz; DMSO-d6): 3.11 (t, 2H); 3.53 (t, 2H); 7.25 (s, 1H); 7.76 (d, 1H); 8.18 (d, 1H); 8.30 (s, 1H); 9.12 (s, 1H); 12.77 (s, 1H).

MS (m/z) ES+: 273 (MH+)

### EXAMPLE 38

### 2-{(E)-2-[4-(2-Hydroxy-2-methyl-propoxy)-phenyl]-vinyl}-8,9,10,11-tetrahydro-3,8,11-triaza-benzo[a]fluoren-7-one

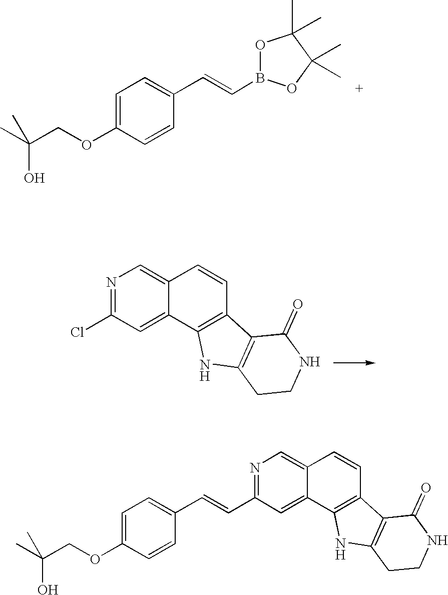

2-Methyl-1-[4-[(E)-2-(4,4,5,5-tetramethyl-[1,3,2]dioxaborolan-2-yl)ethenyl]phenoxy]propan-2-ol (WO 2007039285) is reacted in analogy to example 42. The reaction mixture is evaporated, washed with CH₂Cl₂ and recrystallized from CH₂Cl₂/MeOH to yield the title compound as yellow crystals

1H-NMR (400 MHz; DMSO-d6): 1.25 (s, 6H); 3.13 (t, 2H); 3.78 (s, 2H); 3.55 (m, 2H); 4.66 (s, 1H); 7.01 (d, 2H); 7.20 (bs, 1H); 7.28 (d, 1H); 7.64 (d, 2H); 7.73 (d, 1H); 7.77 (s, 1H); 8.10 (s, 1H); 8.17 (s, 1H); 9.23 (s, 1H); 12.76 (bs, 1H).

MS (m/z) ES+: 428 (MH+).

### Example 39

### 2-[(E)-2-(3-Morpholin-4-yl-phenyl)-vinyl]-8,9,10,11-tetrahydro-3,8,11-triaza-benzo[a]fluoren-7-one

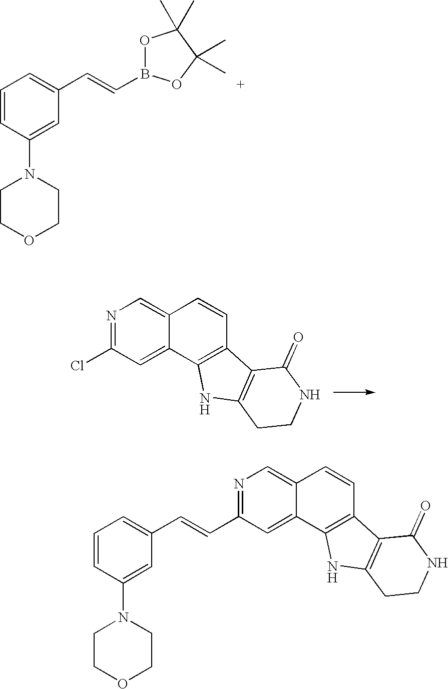

4-{3-[(E)-2-(4,4,5,5-Tetramethyl-[1,3,2]dioxaborolan-2-yl)-vinyl]-phenyl}-morpholine (250 mg; 0.8 mmol) (WO 2004058762) and 2-chloro-8,9,10,11-tetrahydro-3,8,11-triaza-benzo[a]fluoren-7-one (100 mg; 0.37 mmol) are reacted in analogy to example 35 and purified via preparative HPLC-chromatography (Gilson; X-Terra column; acetonitrile/water 4:6 to 1:0). The title compound (containing ˜20% of its cis-isomer) is obtained after recrystallisation from ethanol as yellow crystals.

1H-NMR (400 MHz; DMSO-d6): 3.11 (t, 2H); 3.19 (bt, 4H); 3.54 (bt, 2H); 3.77 (bt, 4H); 6.94 (d, 1H); 7.19 (d, 1H); 7.26-7.29 (m, 2H); 7.40 (d, 1H); 7.60 (bt, 1H); 7.65 (d, 1H); 7.72 (d, 1H); 8.26 (s, 1H); 8.45 (d, 1H); 9.23 (s, 1H); 12.51 (s, 1H).

MS (m/z) ES+: 425 (MH+).

### EXAMPLE 40

### 2-[(E)-2-(3-Morpholin-4-yl-phenyl)-vinyl]-9,10-dihydro-8H-3,8,10-triaza-pentaleno[2,1-a]naphthalen-7-one

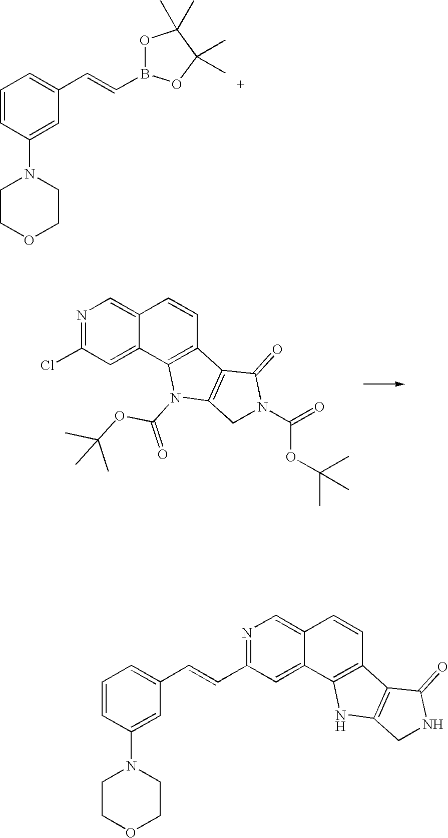

The reaction is performed in analogy to example 25. Purification via chromatography (SiO2; TBME/MeOH/NH₃conc 95:5:1>93:7:1.5) yields the title compound as light-yellow crystals.

1H-NMR (400 MHz; DMSO-d6): 3.21 (m, 4H); 3.79)m, 4H); 4.55 (s, 2H); 6.94 (bd, 1H); 7.20 (d, 1H); 7.27 (m, 2H); 7.42 (d, 1H); 7.79 (m, 3H); 7.87 (d, 1H); 8.29 (s, 1H); 9.29 (s, 1H); 12.98 (bs, 1H).

MS (m/z) ES+: 411 (MH+).

### EXAMPLE 41

### 2-[(E)-2-(3-Morpholin-4-yl-phenyl)-vinyl]-9,10,11,12-tetrahydro-8H-3,8,12-triaza-naphtho[2,1-a]azulen-7-one

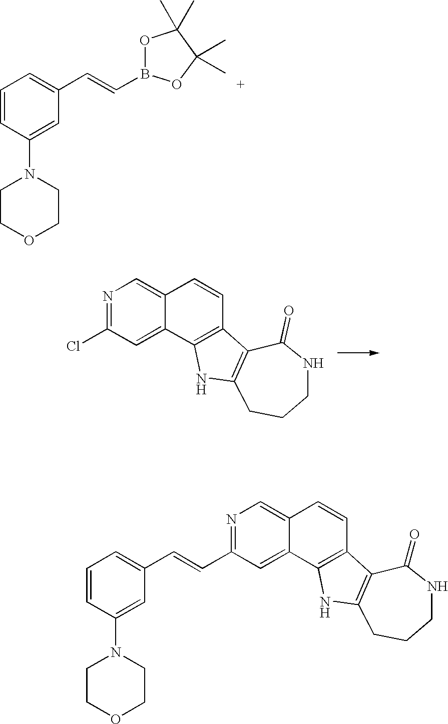

The reaction is performed in analogy to example 35. Purification via chromatography (SiO2; Ethyl acetate/MeOH/NH₃conc 95:5:1) and recrystallisation from methanol yields the title compound as light-yellow crystals (42 mg; 28%). In DMSO-solution the double bond isomerizes within 2 hours into a cis/trans mixture 45:55.

1H-NMR (400 MHz; DMSO-d6): 2.10 (bt, 2H); 3.21 (bt, 4H); 3.28 (bt, 4H); 3.79 (t, 4H); 6.94 (d, 1H); 7.19 (d, 1H); 7.26-7.29 (m, 2H); 7.40 (d, 1H); 7.60 (bt, 1H); 7.65 (d, 1H); 7.72 (d, 1H); 8.26 (s, 1H); 8.45 (d, 1H); 9.23 (s, 1H); 12.51 (s, 1H).

MS (m/z) ES+: 439 (MH+).

### EXAMPLE 42

### 2-[(E)-2-(3-Morpholin-4-ylmethyl-phenyl)-vinyl]-9,10-dihydro-8H-3,8,10-triaza-pentaleno[2,1-a]naphthalen-7-one

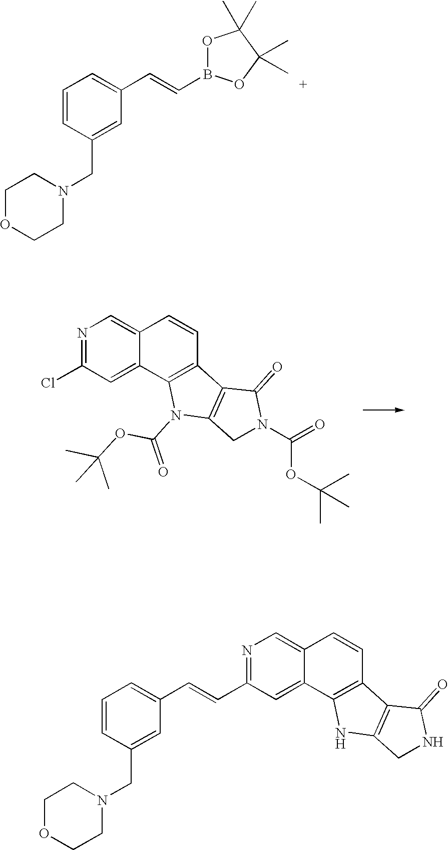

4-{3-[(E)-2-(4,4,5,5-Tetramethyl-[1,3,2]dioxaborolan-2-yl)-vinyl]-benzyl}-morpholine (WO 2004058762) and 2-chloro-7-oxo-7,9-dihydro-3,8,10-triaza-pentaleno[2,1-a]naphthalene-8,10-dicarboxylic acid di-tert-butyl ester are reacted in in analogy to example 25 and purified via chromatography (SiO2; TBME/MeOH/NH₃conc 95:5:1>90:10:2) to yield the title compound as orange crystals.

1H-NMR (400 MHz; DMSO-d6): 2.43 (bs, 4H); 3.55 (s, 2H); 3.62 (bs, 4H); 4.65 (s, 2H); 7.30 (s, 1H); 7.39 (d, 1H); 7.41 (d, 1H); 7.64 (bs, 2H); 7.80-7.88 (m, 4H); 8.31 (s, 1H); 9.31 (s, 1H); 13.05 (bs, 1H).

MS (m/z) ES+: 425 (MH+).

### EXAMPLE 43

### 2-[(E)-2-(3-Morpholin-4-ylmethyl-phenyl)-vinyl]-8,9,10,11-tetrahydro-3,8,11-triaza-benzo[a]fluoren-7-one

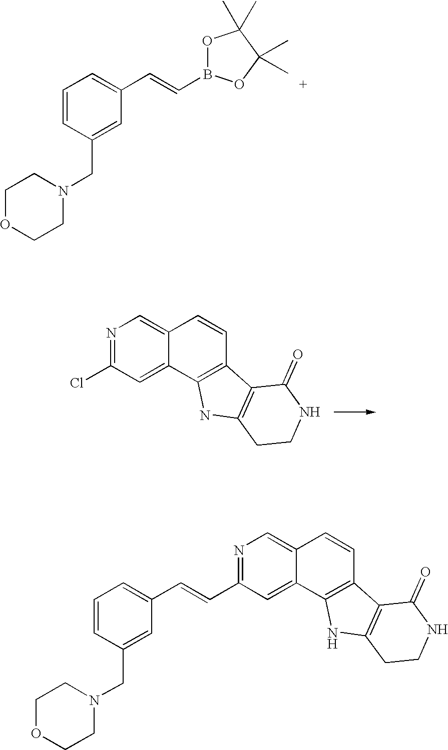

2-Chloro-5,6,8,9,10,11-hexahydro-3,8,11-triaza-benzo[a]fluoren-7-one (100 mg; 0.37 mmol), 4-{3-[(E)-2-(4,4,5,5-tetramethyl-[1,3,2]dioxaborolan-2-yl)-vinyl]benzyl}-morpholine (WO 2004058762) (250 mg; 0.76 mmol), Pd(PPh₃)₂Cl₂ (50 mg; 0.07 mmol), Pd(dppf)₂Cl (20 mg; 0.0002 mmol), PPh₃(80 mg; 0.3 mmol) and 2N Na₂CO₃ (1.1 ml; 2.2 mmol) in 1-propanol (4 ml) are microwaved at 150° C. for 20 minutes. The reaction mixture is filtered and purified via chromatography (SiO₂; TBME/MeOH/NH₃conc 90:10:1) to yield a yellow solid, which yields the title compound after recrystallisation from acetone.

1H-NMR (400 MHz; DMSO-d6): 2.42 (bs, 4H); 3.14 (bt, 2H); 3.54 (bs, 4H); 3.62 (bs, 4H); 7.22 (s, 1H); 7.30 (d, 1H); 7.38-7.42 (m, 2H); 7.63 (bs, 2H); 7.75 (d, 1H); 7.77 (d, 1H); 8.13 (d, 1H); 8.25 (s, 1H); 9.26 (s, 1H); 12.78 (bs, 1H).

MS (m/z) ES+: 440 (MH+).

### EXAMPLE 44

### 2-[(E)-2-(3-Morpholin-4-ylmethyl-phenyl)-vinyl]-9,10,11,12-tetrahydro-8H-3,8,12-triaza-naphtho[2,1-a]azulen-7-one

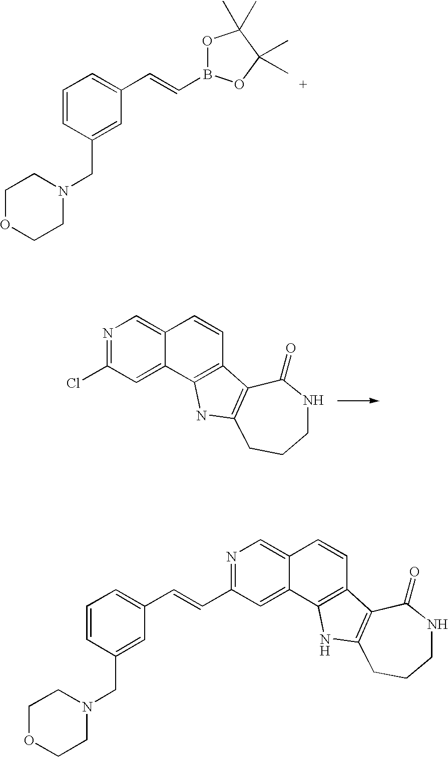

The reaction is performed in analogy to example 43. Purification via chromatography (SIO2; ethyl acetate/MeOH/NH₃conc 95:5:1) yields the title compound after recrystallisation from MeOH as light-yellow crystals.

1H-NMR (400 MHz; DMSO-d6): 2.12 (m, 2H); 2.42 (bs, 2H); 3.29 (bt, 6H); 3.54 (s, 2H); 3.63 (bt, 4H); 7.30 (d, 1H); 7.39 (d, 1H); 7.41 (s, 1H); 7.59-7.63 (m, 3H); 7.70 (d, 1H); 7.75 (d, 1H); 8.28 (s, 1H); 8.45 (d, 1H); 9.24 (s, 1H); 12.52 (s, 1H).

MS (m/z) ES+: 454 (MH+).

### EXAMPLE 45

### 2-{(E)-2-[3-(2-Morpholin-4-yl-ethyl)-phenyl]-vinyl}-8,9,10,11-tetrahydro-3,8,11-triaza-benzo[a]fluoren-7-one

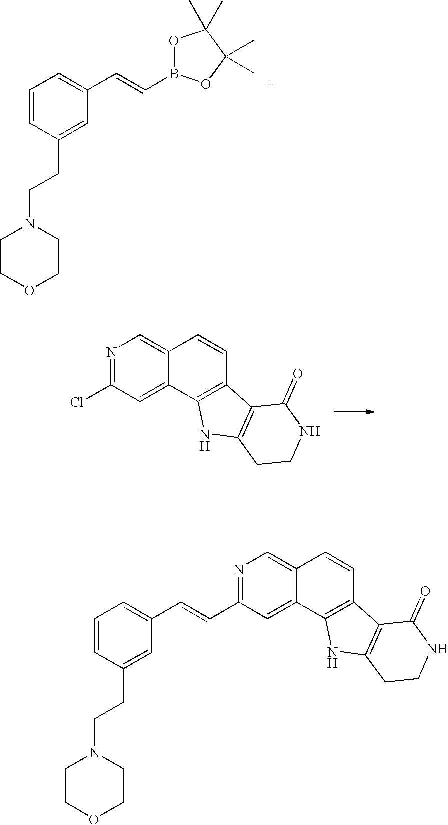

4-(2-{3-[(E)-2-(4,4,5,5-Tetramethyl-[1,3,2]dioxaborolan-2-yl)-vinyl]phenyl}ethyl)-morpholine (WO 2004058762) is reacted in analogy to example 43. Purification via chromatography SiO₂; TBME/MeOH/NH₃conc 90:10:1) followed by recrystallisation from CH₂Cl₂/acetone yields the title compound as light-yellow crystals.

1H-NMR (400 MHz; DMSO-d6): 2.46 (bs, 2H); 2.51 (bs, 2H); 2.58 (bt, 2H); 2.80 (bt, 2H); 3.12 (bt, 2H); 3.54 (bt, 2H); 3.60 (bs, 4H); 7.2 (m, 2H); 7.32 (t, 1H); 7.41 (d, 1H); 7.53 (d, 1H); 7.57 (s, 1H); 7.71 (s, 1H); 7.78 (d, 1H); 8.11 (d, 1H); 8.22 (s, 1H); 9.25 (s, 1H); 12.76 (s, 1H).

MS (m/z) ES+: 454 (MH+).

### EXAMPLE 46

### 2-{(E)-2-[3-(2-Morpholin-4-yl-ethyl)-phenyl]-vinyl}-9,10,11,12-tetrahydro-8H-3,8,12-triaza-naphtho[2,1-a]azulen-7-one

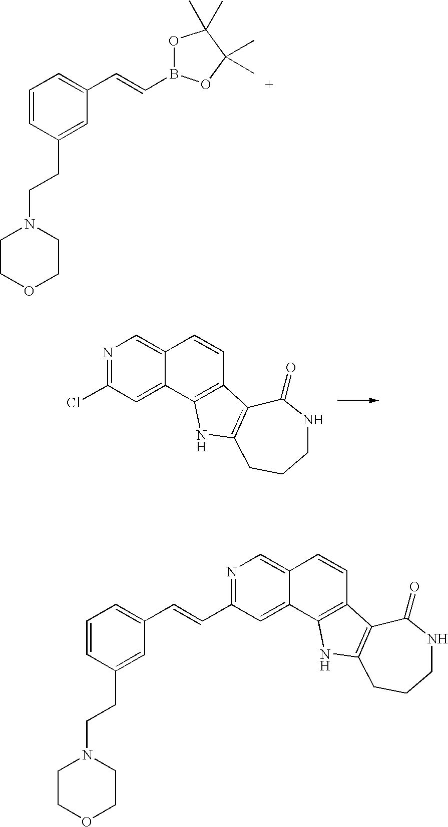

The reaction is performed in analogy to example 43. Purification via chromatography SiO2; ethyl acetate/MeOH/NH₃conc 95:5:0.5) yields the title compound after recrystallisation from MeOH as light-yellow crystals.

1H-NMR (400 MHz; DMSO-d6): 2.07 (bd, 2H); 2.46 (bs, 2H); 2.51 (bs, 2H); 2.57 (bt, 2H); 2.80 (bt, 2H); 3.27 (bt, 4H); 3.60 (bs, 4H); 7.20 (bd 1H); 7.32 (d, 1H); 7.39 (d, 1H); 7.55 (d, 1H); 7.56 (m, 2H); 7.64 (d, 1H); 7.72 (d, 1H); 8.25 (s, 10H); 8.46 (d, 1H); 9.22 (s, 1H); 12.50 (s, 1H).

MS (m/z) ES+: 468 (MH+).

### 1-Morpholin-4-yl-2-(3-trimethylsilanylethynyl-phenyl)-ethanone

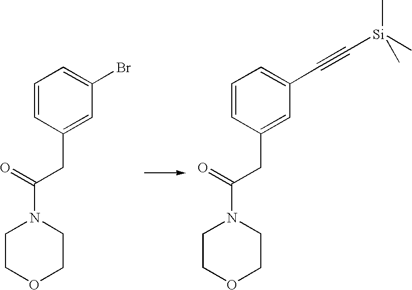

2-(3-Bromo-phenyl)-1-morpholin-4-yl-ethanone morpholine (WO 2004058762) (2.22 g; 7.81 mmol), ethinyltrimethylsilane (10.8 ml; 78.1 mmol), CuBr (199 mg; 1.56 mmol), Pd(PPh₃)₂Cl₂ (274 mg; 0.39 mmol), PPh₃ (2.46 g; 9.37 mmol), triethylamine (18 ml) and DMF (39 ml) are stirred at 150° C. for 2 hours. The reaction mixture is poured on water and extracted with ethyl acetate three times, the organic phases combined, dried over Na₂SO₄ and evaporated to dryness. Chromatography SiO2; Hexanes/acetone 85:15) yields the title compound as yellow crystals.

### 2-(3-Ethynyl-phenyl)-1-morpholin-4-yl-ethanone

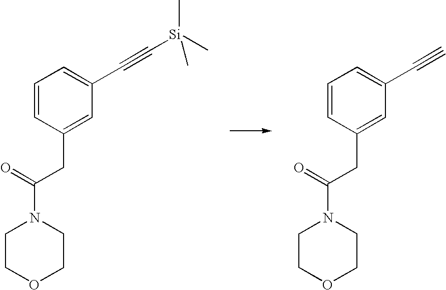

1-Morpholin-4-yl-2-(3-trimethylsilanylethynyl-phenyl)-ethanone (2.1 g; 6.93 mmol) dissolved in EtOH/2N NaOH (20 ml/20 ml) is heated to 55° C. for 30 minutes. The reaction mixture is poured on water and extracted with TBME three times, the organic phases combined, dried over Na₂SO₄ and evaporated to dryness. Chromatography SiO2; Hexanes/acetone 80:20) yields the title compound as yellow crystals.

1H-NMR (400 MHz; DMSO-d6): 3.40-3.55 (m, 8H); 3.75 (s, 2H); 4.15 (s, 1H); 7.24 (m, 1H); 7.35 (m, 3H).

MS (m/z) ES+: 230 (MH+).

### 1-Morpholin-4-yl-2-{3-[(E)-2-(4,4,5,5-tetramethyl-[1,3,2]dioxaborolan-2-yl)-vinyl]-phenyl}-ethanone

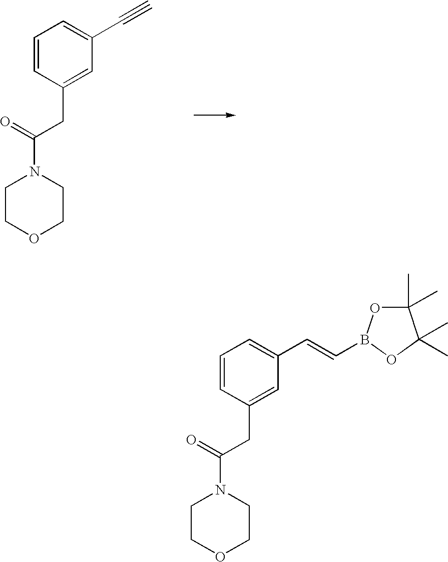

2-(3-Ethynyl-phenyl)-1-morpholin-4-yl-ethanone (906 mg; 3.9 mmol), 4,4,5,5-tetramethyl-[1,3,2]dioxaborolane 1.7 ml; 11.85 mmol) and Rh[P(Ph)₃]₃Cl 70 mg; 0.075 mmol) dissolved in CH₂Cl₂ (35 ml) are stirred over night at room temperature. Chromatography (SiO2; Hexanes/acetone 70:30) yields the title compound as light-yellow resin, which is used in the next step.

### EXAMPLE 47

### 2-{(E)-2-[3-(2-Morpholin-4-yl-2-oxo-ethyl)-phenyl]-vinyl}-9,10-dihydro-8H-3,8,10-triaza-pentaleno[2,1-a]naphthalen-7-one

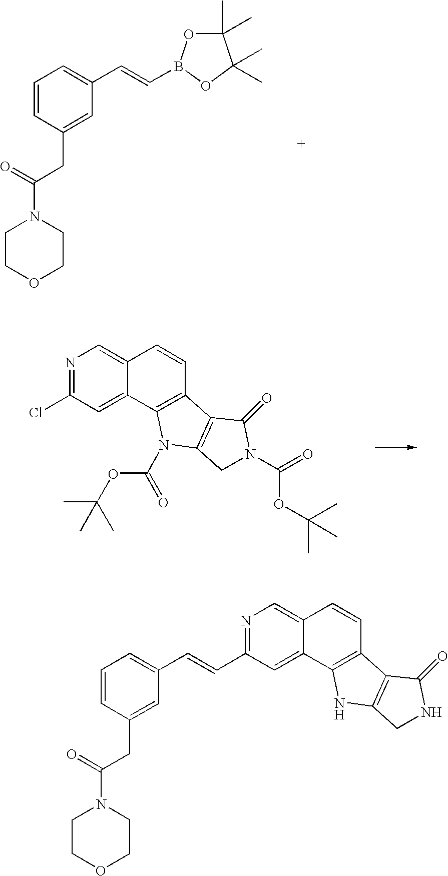

The reaction is performed in analogy to example 25. Purification via chromatography SiO2; TBME/MeOH/NH₃conc 95:5:1>80:20:4) yields the title compound as brown solid.

1H-NMR (400 MHz; DMSO-d6): 3.55 (m, 8H); 3.80 (s, 2H); 4.56 (s, 2H); 7.21 (d, 1H); 7.36 (m, 1H); 7.38 (d, 1H); 7.58 (m, 1H); 7.77 (m, 3H); 7.88 (d, 1H); 8.31 (s, 1H); 9.30 (s, 1H); 13.03 (bs, 1H).

MS (m/z) ES+: 453 (MH+).

### EXAMPLE 48

### 2-{(E)-2-[3-(2-Morpholin-4-yl-2-oxo-ethyl)-phenyl]-vinyl}-8,9,10,11-tetrahydro-3,8,11-triaza-benzo[a]fluoren-7-one

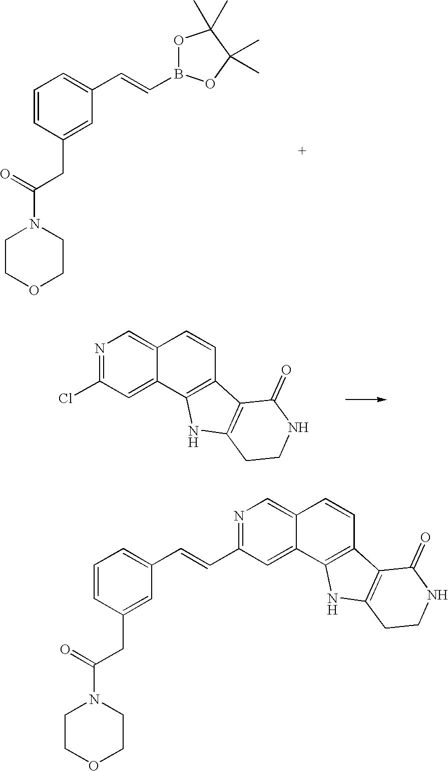

The reaction is performed in analogy to example 43. Purification via chromatography (SiO2; TBME/MeOH/NH₃conc 85:15:1.5) yields the title compound after recrystallisation from MeOH as light-yellow crystals.

1H-NMR (400 MHz; DMSO-d6): 3.14 (t, 2H); 3.51 (m, 2H); 3.56 (m, 8H); 3.80 (s, 2H); 7.19 (m, 2H); 7.38 (m, 2H); 7.57 (s, 1H); 7.60 (d, 1H); 7.73 (s, 1H); 7.79 (d, 1H); 8.13 (d, 1H); 8.25 (s, 1H); 9.26 (s, 1H); 12.76 (s, 1H).

MS (m/z) ES+: 468 (MH+).

### EXAMPLE 49

### 2-{(E)-2-[3-(2-Morpholin-4-yl-2-oxo-ethyl)-phenyl]-vinyl}-9,10,11,12-tetrahydro-8H-3,8,12-triaza-naphtho[2,1-a]azulen-7-one

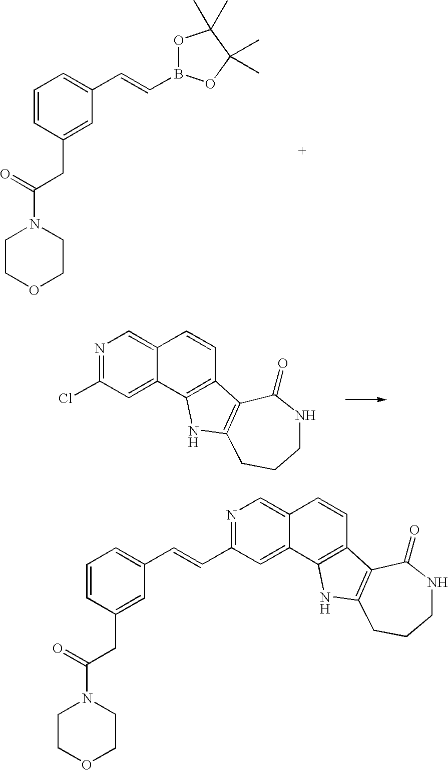

The reaction is performed in analogy to example 43 and purified via chromatography SiO2; CH₂Cl₂/MeOH/NH₃conc 95:5:0.5) followed by recrystallisation from MeOH to yield the title compound as yellow-brown crystals.

1H-NMR (400 MHz; DMSO-d6): 2.12 (m, 2H); 3.26 (m, 6H); 3.49-3.53 (m, 6H); 3.78 (s, 2H); 7.37 (m, 2H); 7.60 (m, 3H); 7.66 (d, 1H); 7.72 (d, 1H); 8.06 (m, 1H); 8.26 (s, 1H); 8.44 (d, 1H); 9.22 (s, 1H); 12.49 (bs, 1H).

MS (m/z) ES+: 482 (MH+).

### 2-(3-Bromo-phenyl)-1-(4-methyl-piperazin-1-yl-ethanone

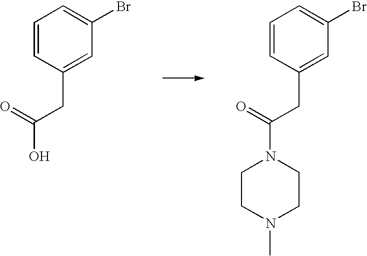

(3-Bromo-phenyl)-acetic acid (4.5 g; 20.9 mmol), EDCl (6.25 g; 32.6 mmol), HOBt (3.32 g; 21.7 mmol), DIPEA (3.5 ml; 20.4 mol) and N-methylpiperazine in CH₂Cl₂ (30 ml) are stirred at room temperature for 1.5 h. The reaction mixture is poured on water and extracted with CH₂Cl₂ three times, the organic phases combined, dried over Na₂SO₄, evaporated to dryness and purified via chromatography SiO₂; CH₂Cl₂/MeOH 95:4) yielding the desired product as brownish crystals.

1H-NMR (400 MHz; DMSO-d6): 2.15 (s, 3H); 2.25 (t, 4H); 3.45 (m, 4H); 3.72 (s, 2H); 7.20 (m, 2H); 7.41 (m, 2H).

MS (m/z) ES+: 298 (MH+).

### 1-(4-Methyl-piperazin-1-yl)-2-(3-trimethylsilanylethynyl-phenyl)-ethanone

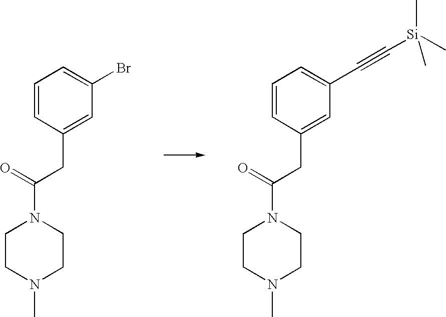

The title compound is prepared in analogy to 1-morpholin-4-y1-2-(3-trimethylsilanylethynyl-phenyl)-ethanone described above and purified via chromatography SiO2; TBME/MeOH/NH₃conc 90:10:2>85:15:3) followed by a second chromatography SiO2; CH₂Cl₂/MeOH/NH₃conc 96:4:0.4) yielding the title compound as light-brown resin.

1H-NMR (400 MHz; DMSO-d6): 0.23 (s, 9H); 2.15 (s, 3H); 2.22 (m, 4H); 3.45 (m, 4H); 3.70 (s, 2H); 7.22 (m, 1H); 7.30 (m, 3H).

MS (m/z) ES+: 316 (MH+).

### 2-(3-Ethynyl-phenyl)-1-(4-methyl-piperazin-1-yl)-ethanone

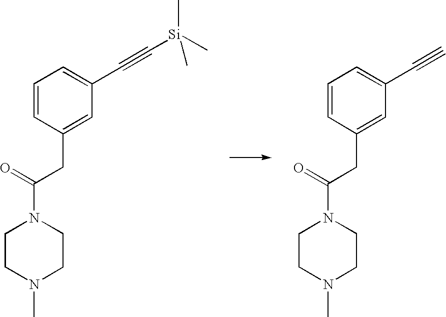

1-(4-Methyl-piperazin-1-yl)-2-(3-trimethylsilanylethynyl-phenyl)-ethanone (1.9 g; 6.07 mmol) in EtOH (20 ml) and 2N NaOH (20 ml) is heated to 50° C. for 30 minutes. The reaction mixture is poured on water and extracted with TBME three times. The organic phases are combined, dried over Na₂SO₄, evaporated to dryness and purified via chromatography SiO2; CH₂Cl₂/MeOH 93:7) to yield the title compound as light-brown crystals.

1H-NMR (400 MHz; DMSO-d6): 2.17 (s, 3H); 2.24 (m, 4H); 3.47 (m, 4H); 3.73 (s, 2H); 4.17 (s, 1H); 7.27 (m, 1H); 7.33 (m, 3H).

MS (m/z) ES+: 243 (MH+).

### 1-(4-Methyl-piperazin-1-yl)-2-{3-[(E)-2-(4,4,5,5-tetramethyl-[1,3,2]dioxaborolan-2-yl)-vinyl]-phenyl}-ethanone

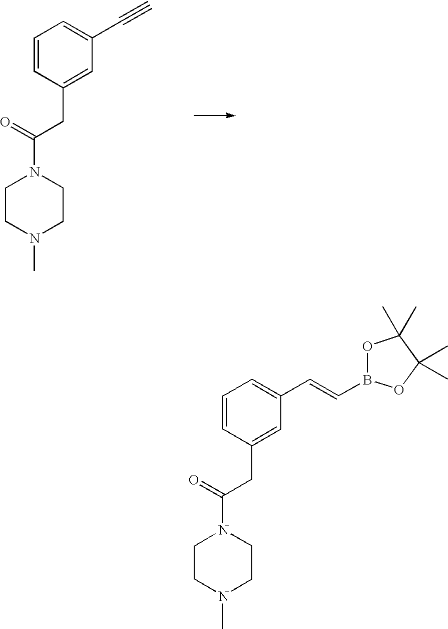

2-(3-Ethynyl-phenyl)-1-(4-methyl-piperazin-1-yl)-ethanone (1.25 g; 5.16 mmol), 4,4,5,5-tetramethyl-[1,3,2]dioxaborolane 2.2 ml; 15.48 mmol) and Rh[P(Ph)₃]₃Cl 70 mg; 0.075 mmol) dissolved in CH₂Cl₂ (50 ml) are stirred over night at room temperature. The reaction mixture is poured on 25% NH₄Cl and extracted with TBME three times. The organic phases are combined, dried over Na₂SO₄, evaporated to dryness and purified via chromatography SiO2; CH₂Cl₂/MeOH 93:7) to yield the title compound as light-brown resin.

### EXAMPLE 50

### 2-((E)-2-{3-[2-(4-Methyl-piperazin-1-yl)-2-oxo-ethyl]-phenyl}-vinyl)-9,10-dihydro-8H-3,8,10-triaza-pentaleno[2,1-a]naphthalen-7-one

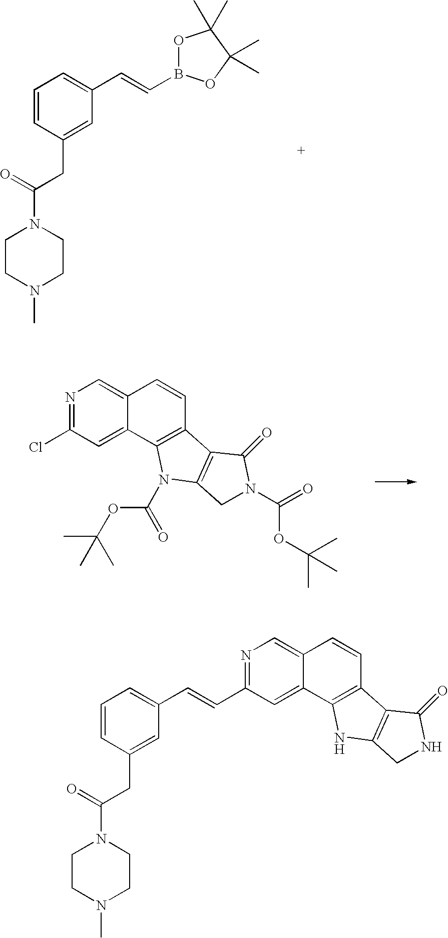

The reaction is performed in analogy to example 25. Purification via chromatography SiO2; CH₂Cl₂/MeOH/NH₃conc 93:7:07 followed by TBME/MeOH/NH₃conc 85:15:3) yielded the title compound as light-brown solid.

1H-NMR (400 MHz; DMSO-d6): 2.18 (s, 3H); 2.26 (m, 4H); 3.52 (m, 4H); 3.79 (s, 2H); 4.56 (s, 2H); 7.20 (d, 1H); 7.40 (m, 3H); 7.58 (s, 1H); 7.80 (m, 3H); 7.85 (d, 1H); 8.31 (s, 1H); 9.31 (s, 1H); 13.03 (bs, 1H).

MS (m/z) ES+: 467 (MH+).

### EXAMPLE 51

### 2-((E)-2-{3-[2-(4-Methyl-piperazin-1-yl)-2-oxo-ethyl]-phenyl}-vinyl)-8,9,10,11-tetrahydro-3,8,11-triaza-benzo[a]fluoren-7-one

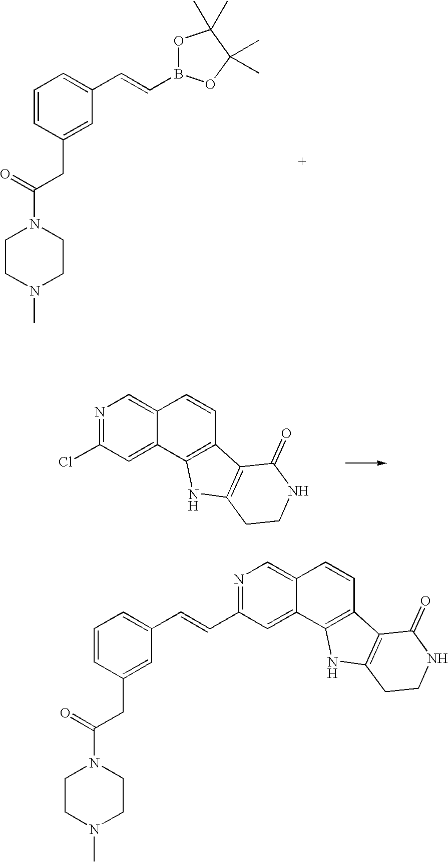

The reaction is performed in analogy to example 43 and purified via preparative HPLC-chromatography (Gilson; X-Terra column; acetonitrile/water 32:68 to 1:0). The title compound is obtained after recrystallisation from MeOH/acetone as yellow crystals.

1H-NMR (400 MHz; DMSO-d6): 2.17 (s, 3H); 2.25 (m, 4H); 3.14 (t, 2H); 3.54 (m, 6H); 3.79 (s, 2H); 7.20 (m, 3H); 7.36 (m, 2H); 7.57 (bs, 1H); 7.73 (d, 1H); 7.79 (s, 1H); 8.13 (d, 1H); 8.25 (s, 1H); 9.26 (s, 1H); 12.76 (bs, 1H).

MS (m/z) ES+: 481 (MH+).

### EXAMPLE 52

### 2-((E)-2-{3-[2-(4-Methyl-piperazin-1-yl)-2-oxo-ethyl]-phenyl}-vinyl)-9,10,11,12-tetrahydro-8H-3,8,12-triaza-naphtho[2,1-a]azulen-7-one

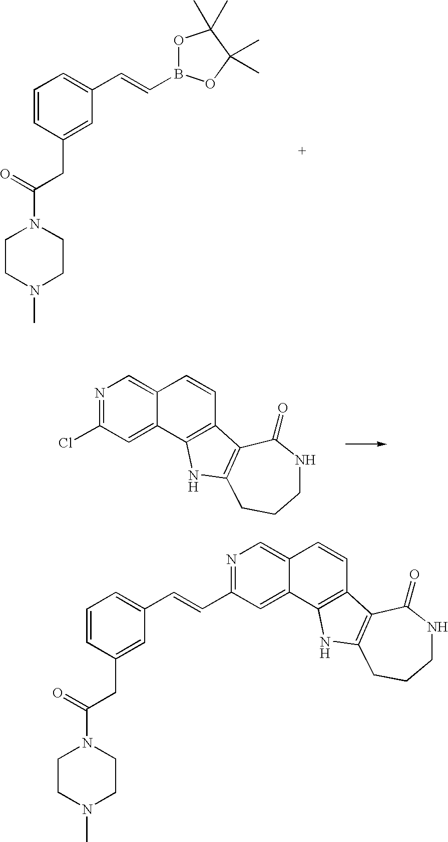

The reaction is performed in analogy to example 43 and purified via chromatography (SiO2; TBME/MeOH/NH₃conc 90:10:1 to 80:20:2) followed by recrystallisation from MeOH to yield the title compound as yellow-brown crystals.

1H-NMR (400 MHz; DMSO-d6): 2.12 (m, 2H); 2.84 (s, 3H); 3.05 (m, 4H); 3.25-3.40 (m, 4H); 3.87 (dd, 2H); 4.25 (bd, 2H); 4.48 (dd, 2H); 7.29 (d, 1H); 7.43 (d, 1H); 7.45 (s, 1H); 7.47 (s, 1H); 7.55 (s, 1H); 7.62 (d, 1H); 7.75 (bs, 1H); 7.83 (d, 1H); 7.93 (d, 1H); 8.65 (d, 1H); 9.55 (bs, 1H); 13.11 (bs, 1H).

MS (m/z) ES+: 495 (MH+).

### EXAMPLE 53

### 11-Methyl-2-[(E)-2-(3-morpholin-4-yl-phenyl)-vinyl]-8,9,10,11-tetrahydro-3,8,11-triaza-benzo[a]fluoren-7-one

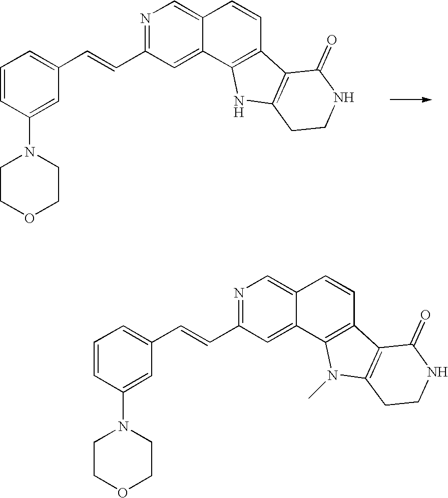

2-[(E)-2-(3-Morpholin-4-yl-phenyl)-vinyl]-8,9,10,11-tetrahydro-3,8,11-triaza-benzo[a]fluoren-7-one (28 mg; 0.067 mmol) dissolved in DMF (7 ml) is treated at 5° C. with a 0.83 N solution of KN(TMS)₂ (0.08 ml; 0.067 mmol) in toluene. After 5 minutes at 5° C. Mel (0.1 ml; 1.6 mmol) is added, the reaction mixture stirred for 20 minutes and purified via chromatography SiO2; CH₂Cl₂/MeOH/NH₃conc 95:5:0.5) followed by recrystallisation from MeOH to yield the title compound as yellow crystals.

1H-NMR (400 MHz; DMSO-d6): 3.16 (t, 2H); 3.21 (m, 4H); 3.57 (m, 2H); 3.80 (bt, 4H); 4.29 (s, 3H); 6.95 (d, 1H); 7.19 (d, 1H); 7.28-7.32 (m, 3H); 7.64 (d, 1H); 7.79 (d, 1H); 7.84 (d, 1H); 8.26 (d, 1H); 8.44 (s, 1H); 9.30 (s, 1H).

MS (m/z) ES+: 439 (MH+).

### Example 54

### 11-Methyl-2-{(E)-2-[3-(2-morpholin-4-yl-ethyl)-phenyl]-vinyl}-8,9,10,11-tetrahydro-3,8,11-triaza-benzo[a]fluoren-7-one

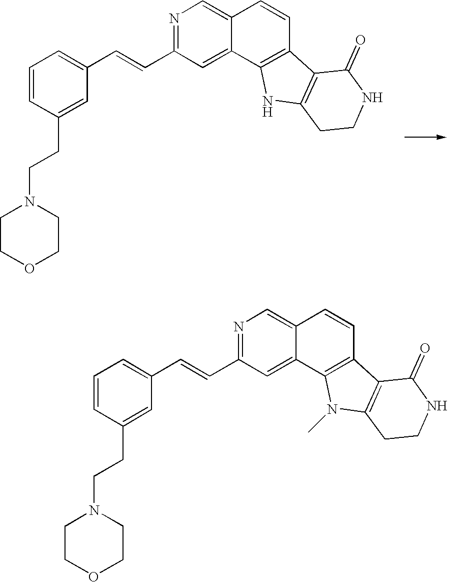

The reaction is performed in analogy to example 53 and purified via chromatography (SiO2; CH₂Cl₂/MeOH/NH₃conc 95:5:0.5)) followed by recrystallisation from MeOH to yield the title compound as colorless crystals.

1H-NMR (400 MHz; DMSO-d6): 2.52 (s, (4H); 2.78 (bt, 2H); 3.17 (t, 2H); 3.57 (bt, 2H); 3.62 (bs, 4H); 4.24 (bt, 2H); 4.36 (s, 3H); 7.05 (bd, 1H); 7.30 (s, 1H); 7.47 (t, 1H); 7.85 (m, 3H); 8.35 (d, 1H); 8.76 (s, 1H); 9.40 (s, 1H).

MS (m/z) ES+: 468 (MH+).

### EXAMPLE 55

### 1,1-Dimethyl-4-(2-{3-[(E)-2-(10-methyl-7-oxo-7,8,9,10-tetrahydro-3,8,10-triaza-pentaleno[2,1-a]naphthalen-2-yl)-vinyl]-phenyl}-acetyl)-piperazin-1-ium iodide

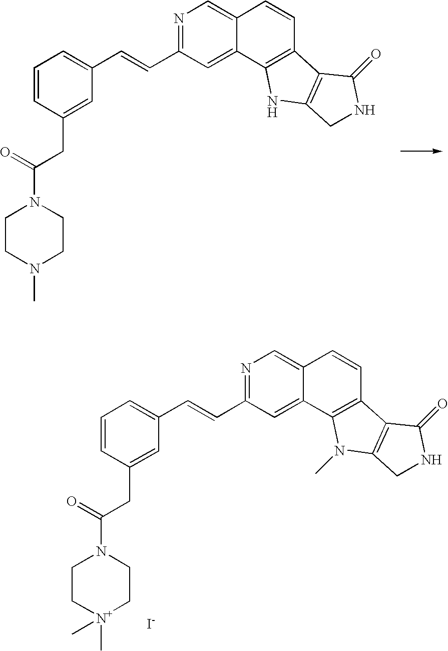

The reaction is performed in analogy to example 53. The reaction mixture is evaporated to dryness and recrystallised from CH₂Cl₂/MeOH to deliver the target compound as yellow solid.

1H-NMR (400 MHz; DMSO-d6): 3.20 (s, 6H); 3.46 (t, 4H); 3.83 (s, 2H); 3.90 (bs, 4H); 4.32 (s, 3H); 4.51 (s, 2H); 7.21 (d, 1H); 7.36 (m, 2H); 7.48-7.56 (m, 3H); 7.82 (m, 2H); 7.92 (d, 1H); 8.49 (s, 1H); 9.28 (s, 1H).

MS (m/z) ES+: 496 (MH+).

### EXAMPLE 56

### 2-[(E)-2-(4-Morpholin-4-ylmethyl-phenyl)-vinyl]-8,9,10,11-tetrahydro-3,8,11-triaza-benzo[a]fluoren-7-one

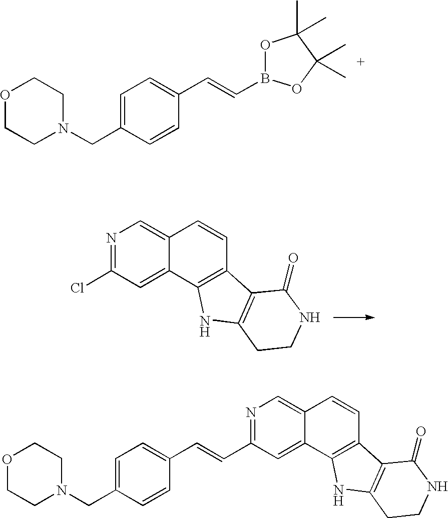

4-[4-[(E)-2-(4,4,5,5-Tetramethyl-[1,3,2]dioxaborolan-2-yl)ethenyl]benzyl]morpholine (WO 2007039285) and 2-chloro-5,6,8,9,10,11-hexahydro-3,8,11-triaza-benzo[a]fluoren-7-one are coupled in analogy to example 43 and purified via chromatography SiO2; TBME/MeOH/NH₃conc 90:10:1 followed by CH₂Cl₂/MeOH/NH₃conc 90:10:1) and recrystallisation from CH₂Cl₂/acetone to yield the title compound as yellow-brown crystals.

1H-NMR (400 MHz; DMSO-d6): 2.38 (bs, 4H); 3.12 (t, 2H); 3.50 (s, 2H); 3.55 (m, 2H); 3.59 (m, 4H); 7.20 (s, 1H); 7.36 (m, 2H); 7.39 (d, 1H); 7.65 (d, 2H); 7.71 (d, 1H); 7.79 (d, 1H); 8.10 (d, 1H); 8.20 (s, 1H); 9.24 (s, 1H); 12.76 (s, 1H).

MS (m/z) ES+: 440 (MH+).

### EXAMPLE 57

### 2-[3-(2-Morpholin-4-yl-ethoxy)-phenyl]-9,10-dihydro-8H-3,8,10-triaza-pentaleno[2,1-a]naphthalen-7-one

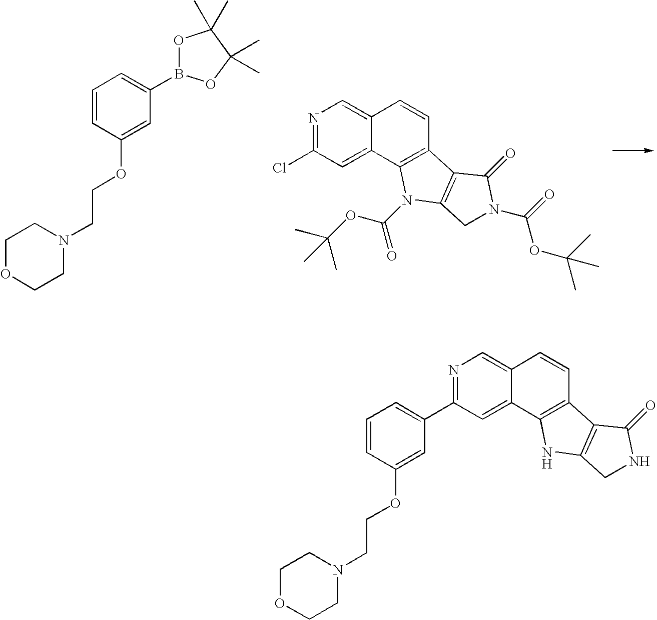

4-[2-[3-(4,4,5,5-Tetramethyl-[1,3,2]dioxaborolan-2-yl)phenoxy]ethyl]morpholine (WO 2004076412) and 2-chloro-7-oxo-7,9-dihydro-3,8,10-triaza-pentaleno[2,1-a]naphthalene-8,10-dicarboxylic acid di-tert-butyl are coupled in analogy to example 25. Purification via chromatography SiO2; TBME/MeOH/NH₃conc 90:10:1.5 followed by CH₂Cl₂/MeOH/NH₃conc 92:8:0.8) and recrystallisation from acetone yields the title compound as yellow crystals.

1H-NMR (400 MHz; DMSO-d6): 2.52 (m, 4H); 2.77 (t, 2H); 3.60 (m, 4H); 4.20 (t, 2H); 4.57 (s, 2H); 7.04 (dd, 1H); 7.46 (t, 1H); 7.75-7.80 (m, 4H); 7.89 (d, 1H); 8.90 (s, 1H); 9.36 (s, 1H); 13.00 (bs, 1H).

MS (m/z) ES+: 429 (MH+).

### EXAMPLE 58

### 2-[3-(2-Morpholin-4-yl-ethoxy)-phenyl]-8,9,10,11-tetrahydro-3,8,11-triaza-benzo[a]fluoren-7-one

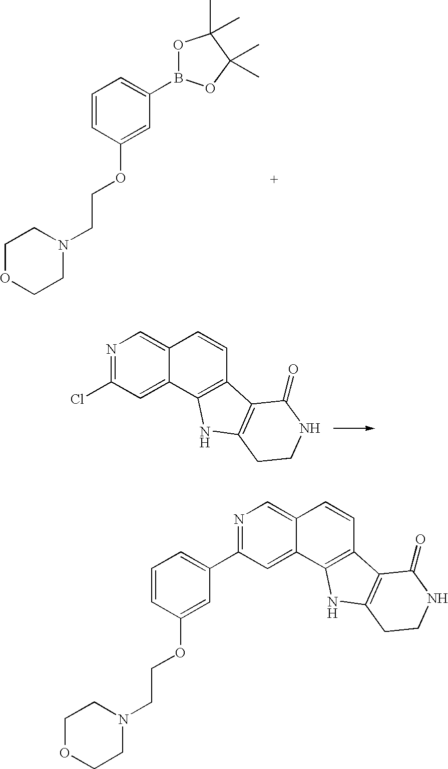

The reaction is performed in analogy to example 43 and purified via chromatography SiO2; TBME/MeOH/NH₃conc 90:10:0 to 85:15:1.5) followed by recrystallisation from MeOH/acetone to yield the title compound as yellow crystals.

1H-NMR (400 MHz; DMSO-d6): 2.55 (m, 4H); 2.78 (t, 2H); 3.16 (t, 2H); 3.57 (bt, 2H); 3.62 (m, 4H); 4.23 (t, 2H); 7.06 (bd, 1H); 7.22 (bs, 1H); 7.47 (t, 1H); 7.74 (d, 1H); 7.80 (bs, 1H); 7.84 (d, 1H); 8.15 (d, 1H); 8.85 (s, 1H); 9.33 (s, 1H); 12.79 (bs, 1H).

MS (m/z) ES+: 444 (MH+).

### EXAMPLE 59

### 2-[3-(2-Morpholin-4-yl-ethoxy)-phenyl]-9,10,11,12-tetrahydro-8H-3,8,12-triaza-naphtho[2,1-a]azulen-7-one

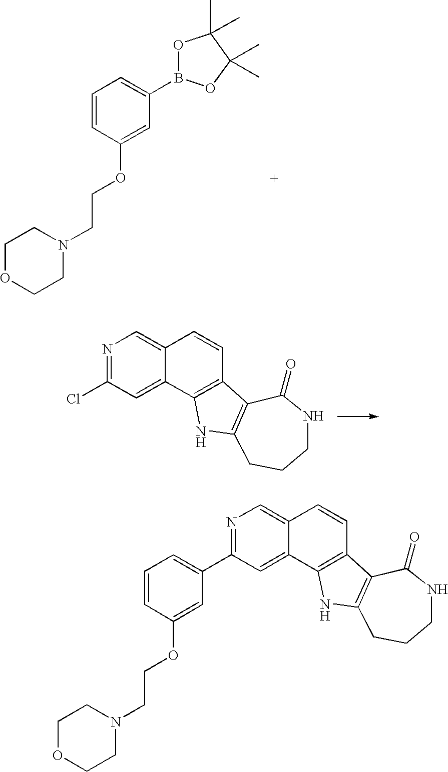

The reaction is performed in analogy to example 43 and purified via chromatography SiO2; TBME/MeOH/NH₃conc 90:10:0 to 85:15:1.5) followed by recrystallisation from MeOH to yield the title compound as yellow crystals.

1H-NMR (400 MHz; DMSO-d6): 2.10 (m, 2H); 2.50 (m, 6H); 2.80 (t, 2H); 3.30 (m, 2H); 3.60 (bt, 4H); 4.20 (t, 2H); 7.00 (dd, 1H); 7.50 (t, 1H); 7.60 (bt, 1H); 7.70 (d, 1H); 7.80 (m, 2H); 8.50 (d, 1H); 8.80 (s, 1H); 9.30 (s, 1H); 12.50 (bs, 1H).

MS (m/z) ES+: 458 (MH+).

### 1-(4-Methyl-piperazin-1-yl)-2-[4-(3,3,4,4-tetramethyl-borolan-1-yl)-phenyl]-ethanone

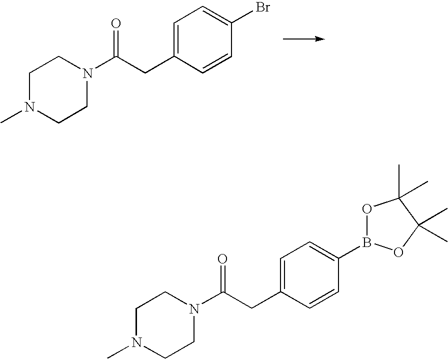

2-(4-Bromo-phenyl)-1-(4-methyl-piperazin-1-yl)-ethanone (100 mg; 0.336 mmol), bis(pinacolato)diboron (130 mg; 0.51 mmol), Pd(dppf)₂Cl (10 mg; 0.012 mmol) and KOAc (99 mg; 1 mmol) are dissolved in DMF (4 ml) and microwaved at 150° C. for 20 minutes. The reaction mixture is diluted with TBME, filtered and evaporated to dryness. The residue is taken up in hot hexane, cooled, filtered and evaporated to deliver the title compound as light-red resin, which is used in the next step.

### EXAMPLE 60

### 2-{4-[2-(4-Methyl-piperazin-1-yl)-2-oxo-ethyl]-phenyl}-8,9,10,11-tetrahydro-3,8,11-triaza-benzo[a]fluoren-7-one

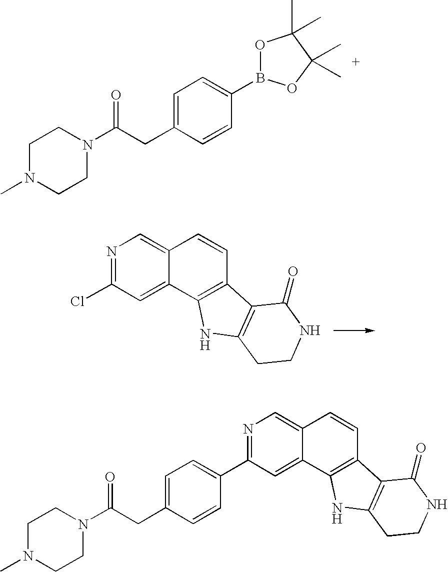

The reaction is performed in analogy to example 43 and purified via chromatography (SiO2; TBME/MeOH/NH₃conc 80:20:0 to 80:20:1.5) followed by recrystallisation from MeOH/acetone to yield the title compound as yellow crystals.

1H-NMR (400 MHz; DMSO-d6): 2.18 (s, 3H); 2.26 (m, 4H); 3.15 (m, 2H); 3.52 (bs, 4H); 3.57 (bt, 2H); 3.82 (s, 2H); 7.21 (bs, 1H); 7.41 (d, 2H); 7.76 (d, 1H); 8.17 (m, 3H); 8.81 (s, 1H); 9.33 (s, 1H); 12.75 (bs, 1H).

MS (m/z) ES+: 455 (MH+).

### 2-(3-Bromo-phenyl)-1-(4-methyl-piperazin-1-yl-ethanone

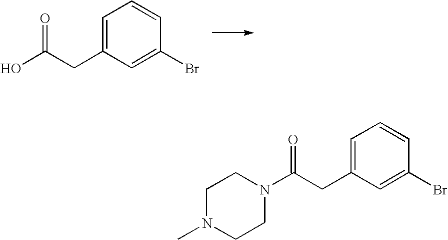

(3-Bromo-phenyl)-acetic acid (1 g; 4.6 mmol) is dissolved in toluene (4 ml), combined with DMF (one drop) and SOCl₂ (2 ml) and refluxed for 10 minutes. The reaction mixture is evaporated, taken up in CH₂Cl₂ (10 ml) and added dropwise to a solution of N-methyl piperazine (1 g; 10 mmol) in CH₂Cl₂. After 5 minutes of stirring at room temperature the reaction mixture The reaction mixture is poured on water and extracted with TBME three times. The organic phases are combined, dried over Na₂SO₄ , filtered and evaporated to dryness, yielding the desired product as light-yellow crystals.

1H-NMR (400 MHz; DMSO-d6): 2.16 (s, 3H); 2.23 (t, 4H); 3.46 (m, 4H); 3.72 (s, 2H); 7.21-7.25 (m, 2H); 7.41 (m, 2H).

MS (m/z) ES+: 298 (MH+).

### 1-(4-Methyl-piperazin-1-yl-2-[3-(4,4,5,5-tetramethyl-[1,3,2]dioxaborolan-2-yl)-phenyl]-ethanone

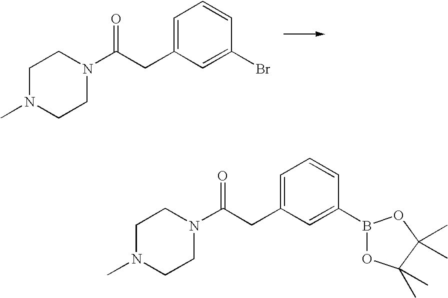

2-(3-Bromo-phenyl)-1-(4-methyl-piperazin-1-yl)ethanone (400 mg; 1.34 mmol), bis(pinacolato)diboron (376 mg; 1.49 mmol), Pd(dppf)₂Cl (40 mg; 0.056 mmol) and KOAc (396 mg; 4.04 mmol) are dissolved in DMF (30 ml) and heated at 150° C. for 20 minutes, then refluxed at 160° C. for 15 minutes. The reaction mixture is evaporated to dryness, taken up in TBME (300 ml) and washed with water (40 ml) twice. The organic phases are combined, dried over Na₂SO₄ , filtered and evaporated to dryness, yielding the desired product as light-brown resin.

### EXAMPLE 61

### 2-{3-[2-(4-Methyl-piperazin-1-yl)-2-oxo-ethyl]-phenyl}-8,9,10,11-tetrahydro-3,8,11-triaza-benzo[a]fluoren-7-one

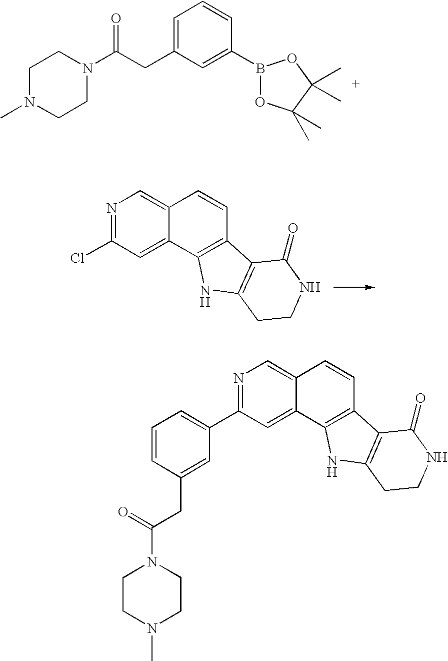

The reaction is performed in analogy to example 43 and purified via chromatography SiO2; TBME/MeOH/NH₃conc 80:20:0 to 80:20:2) followed by recrystallisation from MeOH/CH₂Cl₂ to yield the title compound as yellow crystals.

1H-NMR (400 MHz; DMSO-d6): 2.15 (s, 3H); 2.25 (bs, 4H); 3.13 (m, 2H); 3.53 (m, 6H); 3.83 (s, 2H); 7.21 (s, 1H); 7.29 (d, 1H); 7.49 (t, 1H); 7.75 (d, 1H); 8.04 (d, 1H); 8.10 (s, 1H); 8.13 (d, 1H); 8.78 (s, 1H); 9.32 (s, 1H); 12.80 (bs, 1H).

MS (m/z) ES+: 455 (MH+).

### EXAMPLE 62

### 2-{3-[2(4-Methyl-piperazin-1-yl)-2-oxo-ethyl]-phenyl}-9,10,11,12-tetrahydro-8H-3,8,12-triaza-naphtho[2,1-a]azulen-7-one

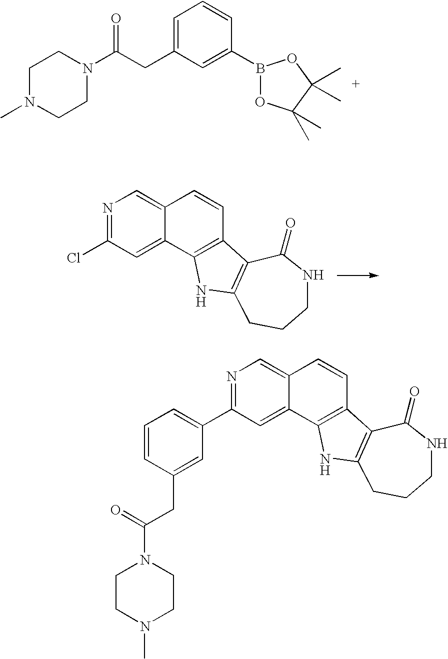

The reaction is performed in analogy to example 43 and purified via chromatography SiO2; TBME/MeOH/NH₃conc 80:20:0 to 80:20:2) followed by recrystallisation from MeOH/acetone to yield the title compound as yellow crystals.

1H-NMR (400 MHz; DMSO-d6): 2.10 (m, 2H); 2.20 (s, 3H); 2.27 (bs, 4H); 3.30 (m, 4H); 3.51 (bd, 4H); 3.87 (s, 2H); 7.30 (d, 1H); 7.50 (t, 1H); 7.61 (bt, 1H); 7.70 (d, 1H); 8.10 (d, 1H); 8.12 (s, 1H); 8.48 (d, 1H); 8.81 (s, 1H); 9.32 (s, 1H); 12.57 (s, 1H).

MS (m/z) ES+: 469 (MH+).

### 4-[2-(5-Bromo-pyridin-3-yloxy)-ethyl]-morpholine

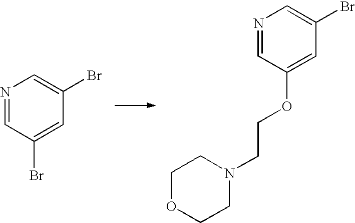

2-Morpholin-4-yl-ethanol (1.11 g; 8.44 mmol) is added at room temperature under stirring to a suspension of NaH (55% in mineral oil; 405 mg; 9.28 mmol) in DMF (20 ml). Stirring is continued for 30 minutes, then 3,5-dibromopyridine (1.0 g; 4.22 mmol) is introduced and the reaction mixture heated to 50° C. for 60 minutes. The reaction mixture is poured on water, extracted with ethyl acetate three times, the organic phases are combined, dried over Na₂SO₄ and evaporated to dryness. Purification via chromatography SiO₂; CH₂Cl₂/MeOH 96:4 to 92:8) yields the title compound as yellow oil.

1H-NMR (400 MHz; DMSO-d6): 2.46 (t, 4H); 2.69 (t, 2H); 3.56 (t, 4H); 4.19 (t, 2H); 7.73 (d, 1H); 8.26 (d, 1H); 8.28 (d, 1H).

MS (m/z) ES+: 288 (MH+).

### 4-{2-[5-(4,4,5,5-Tetramethyl-[1,3,2]dioxaborolan-2-yl)-pyridin-3-yloxy]-ethyl}-morpholine

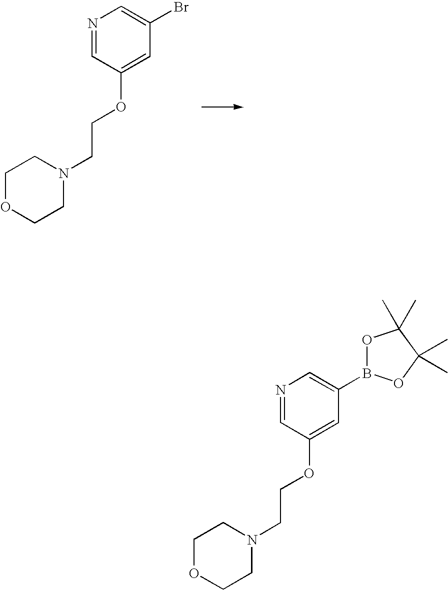

4-[2-(5-Bromo-pyridin-3-yloxy)-ethyl]-morpholine 4.9 g; 17.13 mmol), bis(pinacolato)diboron (8.8 g; 20.56 mmol), Pd(dpdf)₂Cl (391 mg; 0.48 mmol) and KOAc (5.04 g; 51.4 mmol) are dissolved in DMF (150 ml) and heated at 160° C. for 20 minutes. The reaction mixture is evaporated to dryness, taken up in TBME, filtered and evaporated. The title product obtained as red-brown semi-crystalline resin is used in the next step without further purification.

### EXAMPLE 63

### 2-[5-(2-Morpholin-4-yl-ethoxy)-Pyridin-3-yl]-9,10-dihydro-8H-3,8,10-triaza-pentaleno[2,1-a]naphthalen-7-one

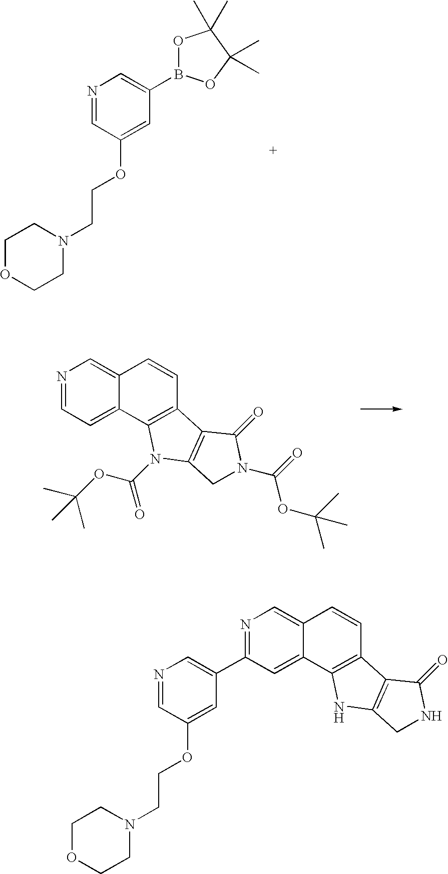

The reaction is performed in analogy to example 25. Purification via chromatography SiO2; CH₂Cl₂/MeOH/NH₃conc 95:5:0.5) yields the title compound as light-brown crystals.

1H-NMR (400 MHz; DMSO-d6): 2.55 (m, 4H); 2.81 (t, 2H); 3.62 (t, 4H); 4.34 (t, 2H); 4.60 (s, 2H); 7.86 (m, 2H); 7.95 (d, 1H); 8.12 (d, 1H); 8.39 (d, 1H); 9.02 (s, 2H); 9.43 (s, 1H); 13.01 (bs, 1H).

MS (m/z) ES+: 430 (MH+).

### EXAMPLE 64

### 2-[5-(2-Morpholin-4-yl-ethoxy)-pyridin-3-yl]-8,9,10,11-tetrahydro-3,8,11-triaza-benzo[a]fluoren-7-one

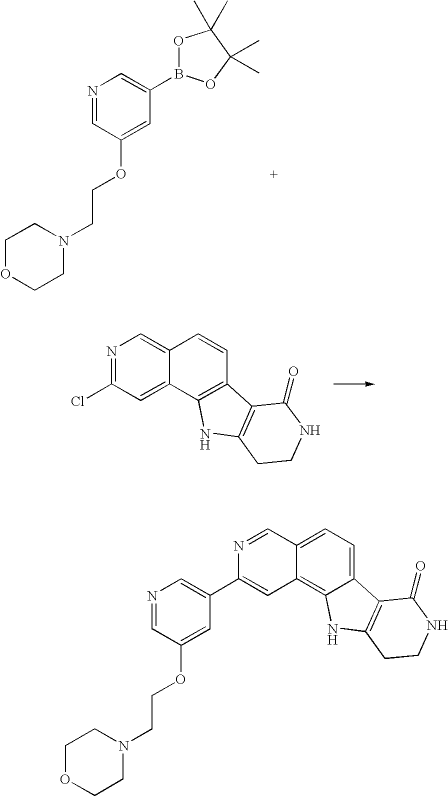

The reaction is performed in analogy to example 43. Purification via chromatography SiO2; TBME/MeOH/NH₃conc 90:10:1) yields the title compound as yellow crystals.

1H-NMR (400 MHz; DMSO-d6): 2.54 (m, 4H); 2.80 (t, 2H); 3.17 (t, 2H); 3.57 (t, 2H); 3.62 (m, 4H); 4.33 (t, 2H); 7.25 (s, 1H); 7.78 (d, 1H); 8.10 (t, 1H); 8.19 (d, 1H); 8.39 (d, 1H); 8.93 (s, 1H); 9.00 (s, 1H); 9.38 (s, 1H); 12.74 (bs, 1H).

MS (m/z) ES+: 444 (MH+).

### EXAMPLE 65

### 2-[5-(2-Morpholin-4-yl-ethoxy)-pyridin-3-yl]-9,10,11,12-tetrahydro-8H-3,8,12-triaza-naphtho[2,1-a]azulen-7-one

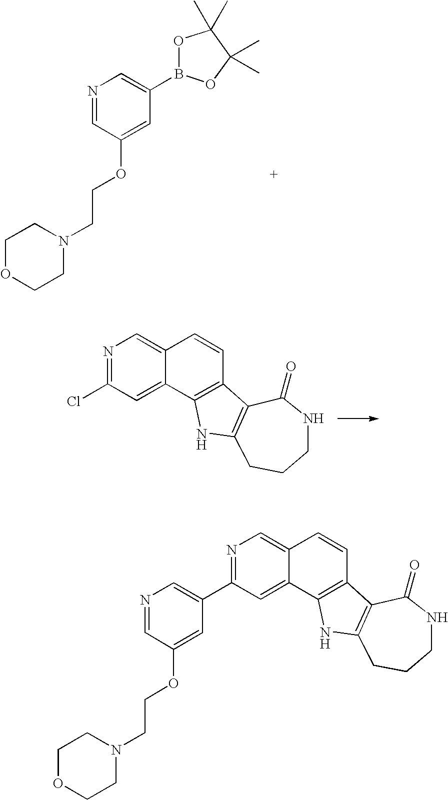

The reaction is performed in analogy to example 43. Purification via chromatography SiO2; TBME/MeOH 85:15) yields the title compound as yellow crystals.

1H-NMR (400 MHz; DMSO-d6): 2.12 (m, 2H); 2.55 (m, 4H); 2.80 (t, 2H); 3.31 (m, 4H); 3.62 (t, 4H); 4.33 (t, 2H); 7.63 (bs, 1H); 7.73 (d, 1H); 8.10 (d, 1H); 8.38 (d, 1H); 8.52 (d, 1H); 8.94 (s, 1H); 9.01 (s, 1H); 9.36 (s, 1H); 12.56 (bs, 1H).

MS (m/z) ES+: 459 (MH+).

### 5-Bromo-3-(2-methoxy-ethoxy)-pyridine

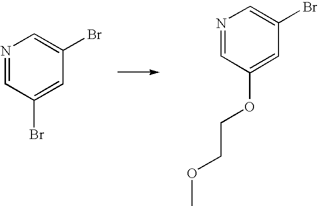

2-Methoxyethanol (2.7 ml; 33.8 mmol) is added dropwise to a suspension of NaH (55% suspension; 1.62 g; 37.14 mmol) in DMF (60 ml). After stirring for 30 minutes 3,5-dibromopyridine (4.0 g; 16.88 mmol) is introduced and the mixture heated to 50° C. for 1 hour. The reaction mixture is poured on water and extracted with ethyl acetate three times, the organic phases combined, dried over Na₂SO₄ and evaporated to dryness. Chromatography (SiO₂; Hexanes/acetone 85:15) yields the title compound as yellow solid.

1H-NMR (400 MHz; DMSO-d6): 8.31 (d, 1H); 8.28 (d, 1H); 7.73 (t, 1H); 4.23 (dd, 2H); 3.67 (dd, 2H); 3.32 (s, 3H).

MS (m/z) ES+: 232, 234 (MH+).

### 3-(2-Methoxy-ethoxy)-5-(4,4,5,5-tetramethyl-[1,3,2]dioxaborolan-2-yl)-pyridine

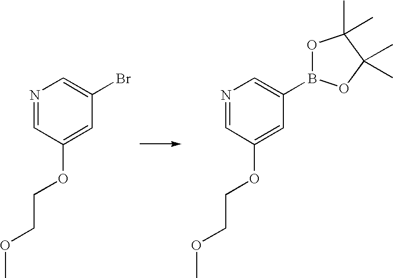

5-Bromo-3-(2-methoxy-ethoxy)-pyridine (6.7 g; 28.9 mmol), bis(pinacolato)diboron (8.8 g; 34.7 mmol), Pd(dppf)₂Cl (660 mg; 0.81 mmol) and KOAc (8.5 g; 86.7 mmol) in DMF (240 ml) are heated to 160° C. for 20 minutes. The reaction mixture is evaporated, dissolved in TBME, filtered and evaporated again to deliver the target compound as a semi-crystalline red-brown solid, which is used in the next step without further purification.

### EXAMPLE 66

### 2-[5-(2-Methoxy-ethoxy)-pyridin-3-yl]-9,10,11,12-tetrahydro-8H-3,8,12-triaza-naphtho[2,1-a]azulen-7-one

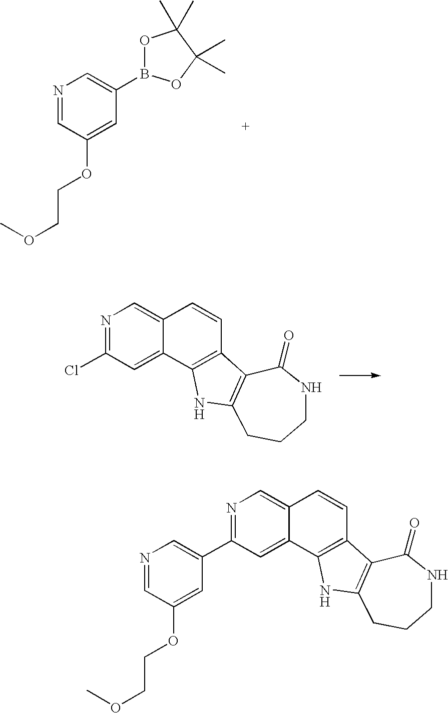

The reaction is performed in analogy to example 43. Purification via chromatography SiO2; TBME/MeOH 80:10 to TBME/MeOH/NH₃conc 85:15:1.5) yields the title compound as yellow crystals.

1H-NMR (400 MHz; DMSO-d6): 2.11 (m, 2H); 3.36 (s, 3H); 3.30 (m, 4H); 3.76 (m, 2H); 4.34 (m, 2H); 7.64 (t, 1H); 7.73 (d, 1H); 8.10 (d, 1H); 8.38 (d, 1H); 8.52 (d, 1H); 8.94 (s, 1H); 9.01 (s, 1H); 9.36 (s, 1H); 12.50 (bs, 1H).

MS (m/z) ES+: 403 (MH+).

### EXAMPLE 67

### 2-[5-(2-Methoxy-ethoxy)-pyridin-3-yl]-9,10-dihydro-8H-3,8,10-triaza-pentalen[2,1-a]naphthalen-7-one

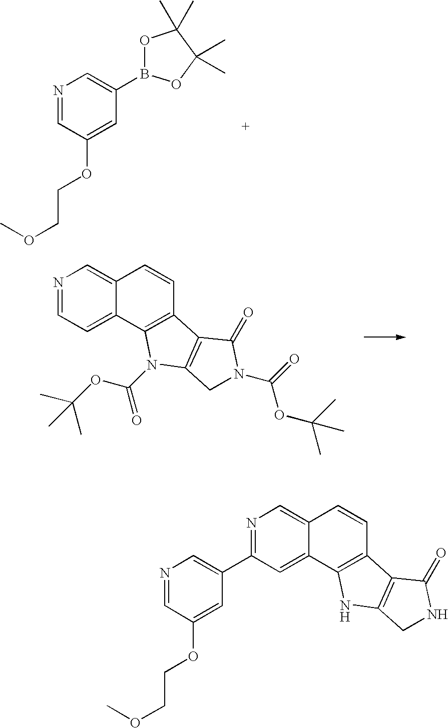

The reaction is performed in analogy to example 25. Purification via chromatography (SiO2; TBME/MeOH 80:10 to TBME/MeOH/NH₃conc 85:15:1) yields the title compound as yellow crystals.

1H-NMR (400 MHz; DMSO-d6): 3.36 (s, 3H); 3.75 (m, 2H); 4.34 (m, 2H); 4.58 (s, 2H); 7.81 (d, 1H); 7.83 (s, 1H); 7.93 (d, 1H); 8.09 (m, 1H); 8.40 (d, 1H); 8.99 (d, 2H); 9.41 (s, 1H); 12.93 (bs, 1H).

MS (m/z) ES+: 375 (MH+).

### EXAMPLE 68

### 2-[5-(2-Methoxy-ethoxy)-pryidin-3-yl]-8,9,10,11-tetrahydro-3,8,11-triaza-benzo[a]fluoren-7-one

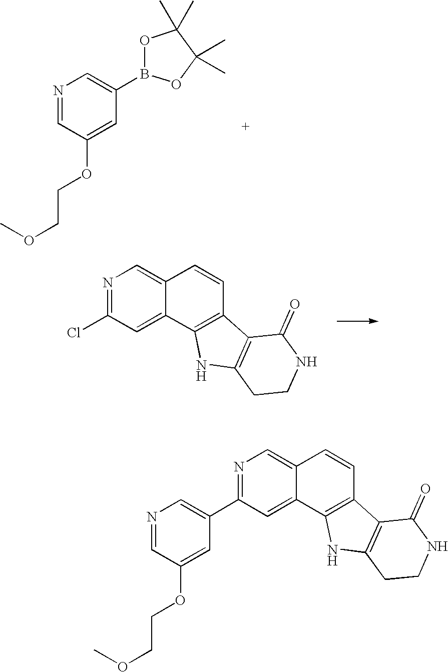

The reaction is performed in analogy to example 43. Purification via chromatography SiO2; TBME/MeOH 80:10 to TBME/MeOH/NH₃conc 90:10:1) yields the title compound as yellow crystals.

1H-NMR (400 MHz; DMSO-d6): 3.13 (m, 2H); 3.32 (s, 3H); 3.53 (m, 2H); 3.72 (m, 2H); 4.32 (m, 2H); 7.22 (bs, 1H); 7.78 (d, 1H); 8.00 (s, 1H); 8.19 (d, 1H); 8.35 (s, 1H); 8.93 (s, 1H); 8.98 (s, 1H); 9.40 (s, 1H); 12.8 (s, 1H).

MS (m/z) ES+: 389 (MH+).

### EXAMPLE 69

### 2-Pyridin-3-yl-9,10,11,12-tetrahydro-8H-3,8,12-triaza-naphtho[2,1-a]azulen-7-one

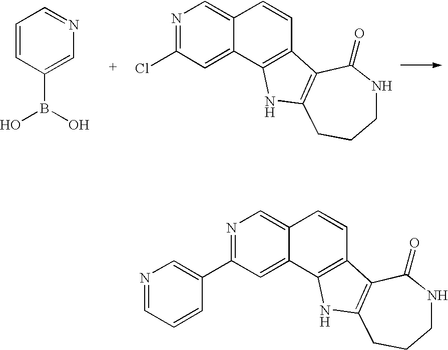

The reaction is performed in analogy to example 23. Purification via chromatography SiO2; acetone/hexane 80:20 to acetone/MeOH 95:5) followed by recrystallisation from MeOH/CH₂Cl₂ yields the title compound as yellow crystals.

1H-NMR (400 MHz; DMSO-d6): 2.08 (m, 2H); 3.29 (m, 4H); 7.57 (m, 2H); 7.72 (d, 1H); 8.50 (m, 2H); 8.62 (d, 1H); 8.94 (s, 1H); 9.35 (s, 1H); 9.38 (d, 1H); 12.60 (bs, 1H).

MS (m/z) ES+: 329 (MH+).

### EXAMPLE 70

### 2-(5-Methoxy-pyridin-3-yl)-9,10-dihydro-8H-3,8,10-triaza-pentaleno[2,1-a]naphthalen-7-one

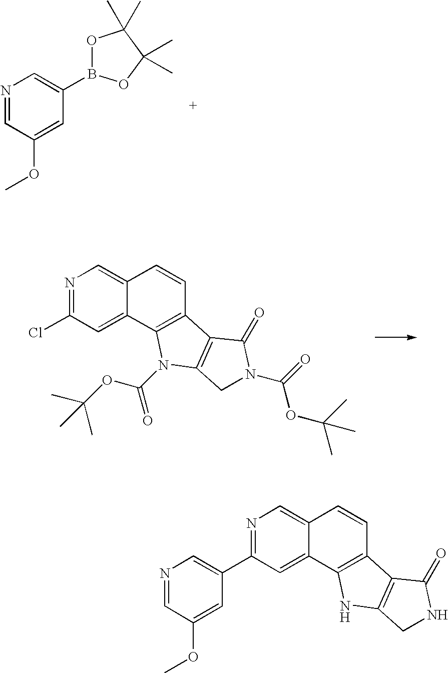

The reaction is performed in analogy to example 23. Purification via chromatography SiO2; ethyl acetate/hexane 6:4 to ethyl acetate/MeOH/NH₃conc 92:8:0.8) followed by recrystallisation from MeOH/CH₂Cl₂ yields the title compound as light-brown crystals.

1H-NMR (400 MHz; DMSO-d6): 3.99 (s, 3H); 4.60 (s, 2H); 7.85 (d, 1H); 7.88 (s, 1H); 7.95 (d, 1H); 8.11 (s, 1H); 8.38 (s, 1H); 9.00 (s, 2H); 9.43 (s, 1H); 13.00 (bs, 1H).

MS (m/z) ES+: 331 (MH+).

### EXAMPLE 71

### 2-(6-Methoxy-pyridin-3-yl)-9,10-dihydro-8H-3,8,10-triaza-pentaleno[2,1-a]naphthalen-7-one

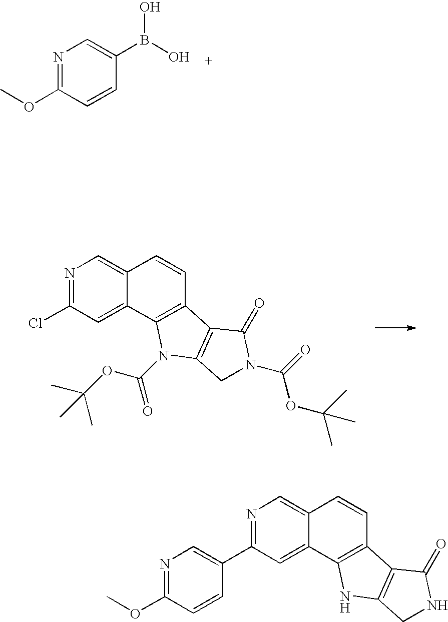

The reaction is performed in analogy to example 23. Purification via chromatography SiO2; ethyl acetate/hexane 6:4 to ethyl acetate/MeOH/NH₃conc 92:8:0.8) followed by recrystallisation from MeOH/CH₂Cl₂ yields the title compound as light-brown crystals.

1H-NMR (400 MHz; DMSO-d6): 3.97 (s, 3H); 4.48 (s, 2H); 7.03 (d, 1H); 7.56 (bs, 1H); 7.69 (d, 1H); 7.87 (d, 1H); 8.44 (bd, 1H); 8.81 (s, 1H); 8.98 (s, 1H); 9.31 (s, 1H); 13.5 (s, 1H).

MS (m/z) ES+: 331 (MH+).

### EXAMPLE 72

### 2-(6-Dimethylamino-pyridin-3-yl)-9,10-dihydro-8H-3,8,10-triaza-pentaleno[2,1-a]naphthalen-7-one

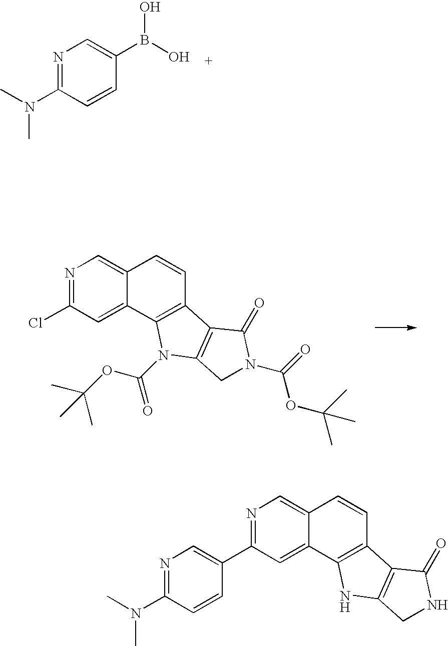

The reaction is performed in analogy to example 23. The reaction mixture is diluted with CH₂Cl₂/MeOH (2:1) and the precipitated product filtered, washed successively with water, MeOH and TBME to yield the title product as light-brown crystals.

1H-NMR (400 MHz; DMSO-d6): 3.14 (s, 6H); 4.58 (s, 2H); 6.85 (d, 1H); 7.76 (d, 1H); 7.82 (bs, 1H); 7.86 (d, 1H); 8.27 (d, 1H); 8.72 (s, 1H); 8.96 (s, 1H); 9.31 (s, 1H); 12.92 (bs, 1H).

MS (m/z) ES+: 344 (MH+).

### EXAMPLE 73

### 2-{(E)-2-[4-(2-Hydroxy-2-methyl-propoxy)-phenyl]-vinyl}-9,10,11,12-tetrahydro-8H-3,8,12-triaza-naphtho[2,1-a]azulen-7-one

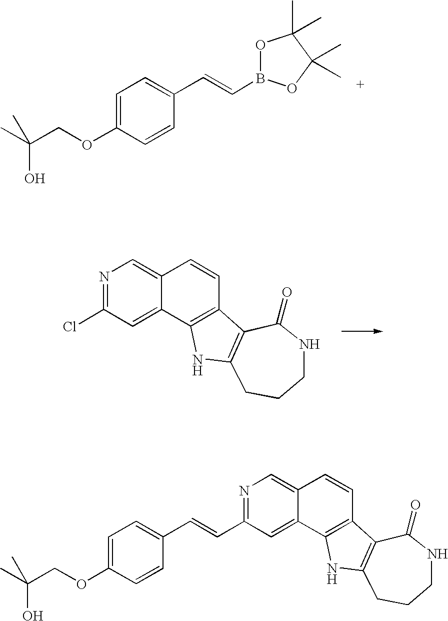

2-Methyl-1-[4-[(E)-2-(4,4,5,5-tetramethyl-[1,3,2]dioxaborolan-2-yl)ethenyl]phenoxy]propan-2-ol (WO 2007039285) is reacted in analogy to example 43. Purification via chromatography SiO2; EtOAc/MeOH/NH₃conc 90:10:1) and crystallization from CH₂Cl₂/MeOH yields the title compound as light-yellow crystals.

1H-NMR (400 MHz; DMSO-d6): 1.24 (s, 6H); 2.10 (m, 2H); 3.29 (m, 4H); 3.78 (s, 2H); 4.66 (s, 1H); 7.01 (d, 2H); 7.26 (d, 1H); 7.59 (m, 1H); 7.64 (m, 3H); 7.72 (d, 1H); 8.20 (s, 1H); 8.43 (d, 1H); 9.21 (s, 1H); 12.5 (bs, 1H).

MS (m/z) ES+: 443 (MH+).

### EXAMPLE 74

### 2-(3-Fluoro-4-methoxy-phenyl)-8,9,10,11-tetrahydro-3,8,11-triaza-benzo[a]fluoren-7-one

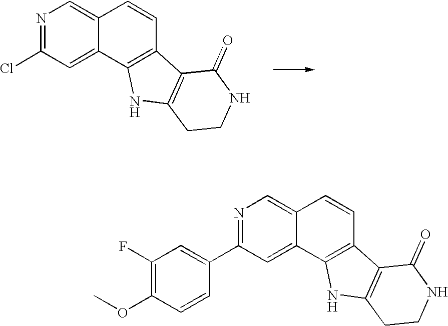

2-Chloro-8,9,10,11-tetrahydro-3,8,11-triaza-benzo[a]fluoren-7-one (Example 37; 140 mg; 0.55 mmol), 3-fluoro-4-methoxyphenylboronic acid (140 mg; 0.83 mmol), Pd(PPh₃)₂Cl₂ (39 mg; 0.055 mmol), in DMF/2N Na₂CO₃ solution (2 ml/0.5 ml) are heated to 160° C. in a microwave oven for 0.5 h. The reaction mixture is purified via reversed phase HPLC (Waters X-Terra; acetonitrile/water) to deliver the title compound.

1H-NMR (400 MHz; DMSO-d6): 3.15 (t, 2H), 3.57 (dt, 2H), 3.95 (s, 3H), 7.24 (s, 1H), 7.39 (t, 1H), 7.75 (d, 1H), 8.00-8.15 (m, 2H), 8.16 (d, 1H), 8.80 (s, 1H), 9.33 (s, 1H), 12. 75 (bs, 1H).

MS (m/z) ES+: 362 (MH+)

### EXAMPLE 75

### 2-(3-Chloro-4-propoxy-phenyl)-8,9,10,11-tetrahydro-3,8,11-triaza-benzo[a]fluoren-7-one

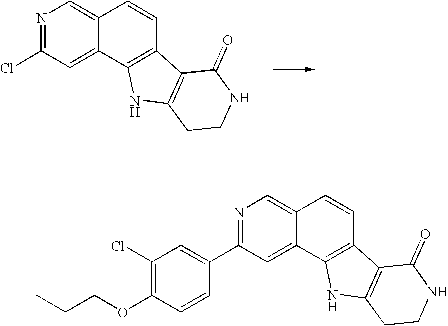

Using 2-Chloro-8,9,10,11-tetrahydro-3,8,11-triaza-benzo[a]fluoren-7-one (Example 37; 150 mg; 0.55 mmol) and 3-chloro-4-propoxyphenylboronic acid (178 mg; 0.83 mmol) the title compound is prepared in analogy to example 74.

1H-NMR (400 MHz; DMSO-d6): 1.06 (t, 3H), 1.83 (sext, 2H), 3.15 (t, 2H), 3.56 (td, 2H), 4.14 (t, 2H), 7.23 (s, 1H), 7.37 (d, 1H), 7.75 (d, 1H), 8.10-8.20 (m, 2H), 8.26 (s, 1H), 8.80 (s, 1H), 9.33 (s, 1H), 12.76 (bs, 1H).

MS (m/z) ES+: 406 (MH+)

### EXAMPLE 76

### 2-(3-Fluoro-4-methoxy-phenyl)-9,10-dihydro-8H-cyclopenta[4,5]pyrrolo[2,3-f]isoquinolin-7-one

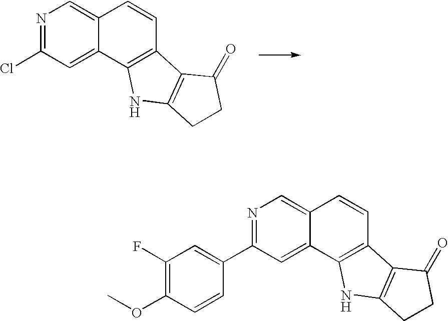

Following the procedure described in example 74 the title compound is obtained by reacting 2-chloro-9,10-dihydro-8H-cyclopenta[4,5]pyrrolo[2,3-f]isoquinolin-7-one (example 37) and 3-fluoro-4-methoxyphenylboronic acid.

1H-NMR (400 MHz; DMSO-d6): 2.93 (dd, 2H), 3.26 (dd, 2H), 3.96 (s, 3H), 7.41 (t, 1H). 7.84 (d, 1H), 7.94 (d, 1H), 8.00-8.15 (m, 20H), 8.85 (s, 1H), 9.39 (s, 1H), 13.11 (bs, 1H).

MS (m/z) ES+: 345 (M−H)⁻

### EXAMPLE 77

### 2-(3-Chloro-4-propoxy-phenyl)-9,10-dihydro-8H-cyclopenta[4,5]pyrrolo[2,3-f]isoquinolin-7-one

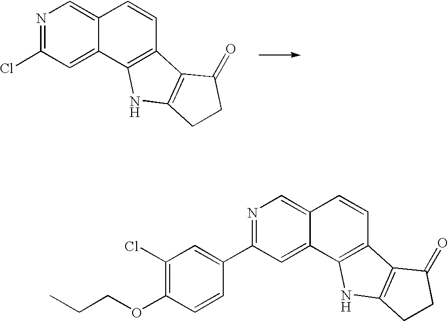

Following the procedure described in example 74 the title compound is obtained by reacting 2-chloro-9,10-dihydro-8H-cyclopenta[4,5]pyrrolo[2,3-f]isoquinolin-7-one (example 37) and 3-chloro-4-propoxyphenylboronic acid.

1H-NMR (400 MHz; DMSO-d6): 1.06 (t, 3H), 1.83 (sext, 2H), 2.93-2.97 (m, 2H), 3.23-3.28 (m, 2H), 4.15 (t, 2H), 7.38 (d, 1H), 7.84 (d, 1H), 7.93 (d, 1H), 8.18 (dd, 1H), 8.27 (d, 1H), 8.85 (s, 1H), 9.39 (s, 1H), 13.1 (bs, 1H).

MS (m/z) ES+: 391 (MH⁺)

Agents of the Invention possess MAPKAPK2 (MAP Kinase Activated Protein Kinase) inhibiting activity. Thus the Agents of the Invention act to inhibit production of inflammatory cytokines, such as TNF-α, and also to potentially block the effects of these cytokines on their target cells. These and other pharmacological activities of the Agents of the Invention as may be demonstrated in standard test methods for example as described below:

MAPKAPK2 Kinase Assay

MAPAPK2 is pre-activated in kinase buffer (25 mM TRIS-HCL, pH 7.5, 25 mM beta-glycerophosphate, 0.1 mM sodium orthovanadate, 25 mM MgCl₂, 20 μM DTT) containing 5 μM ATP, 150 μg/ml human MK2 (HPLC purified in house), 30 μg/ml active human p38α (HPLC purified in house) for 30 min at 22 ° C. For the measurement of compound inhibition on activated MAPAPK2, each reaction contained test compound (10 μl; 0.5% DMSO final) or vehicle control, 250 nM Hsp27 peptide biotinyl-AYSRALSRQLSSGVSEIR—COOH as substrate (10 μl) and pre-activated MAPKAP2 kinase mix (10 μl) containing ATP (5 μM final). To define non-specific, reactions are performed in the absence of substrate. Following incubation at 22° C. for 45 min, kinase reactions are terminated with 125 μM EDTA (10 μl). Samples (10 μl) are transferred to black low volume 384-well plates (Greiner) prior to the detection of phosphorylated substrate by time-resolved fluorescence resonance energy transfer (TR-FRET). Phosphorylated Hsp27 is measured using an antibody mix (10 μl) containing a rabbit anti-phospho-Hsp27 (Ser⁸²) antibody (2.5 nM, Upstate) in conjunction with an anti-rabbit europium-labeled secondary antibody LANCE Eu-W1024 (2.5 nM; Perkin Elmer) as fluorescence donor along with streptavidin SureLight-APC (6.25 nM; Perkin Elmer) as a fluorescence acceptor. Following incubation at 22 ° C. for 90 min, plates are measured at 615 and 665 nm using a PHERAstar (BMG Labtech). The 615/665 nm ratio is determined following subtraction of background. Values are expressed as % inhibition using control values. Individual IC₅₀ values of compounds are determined by nonlinear regression after fitting of curves to the experimental data using Excel XL fit 4.0 (Microsoft).

Assay for Inhibition of TNF-α Release from hPBMCs

Human peripheral blood mononuclear cells (hPBMCs) are prepared from the peripheral blood of healthy volunteers using Ficoll-Plaque Plus (Amersham) density separation according to the method described within. Cells are seeded at a 1×10⁵ cells/well in 96-well plates in RPMI 1640 medium (Invitrogen) containing 10% (v/v) fetal calf serum (FCS). Cells are pre-incubated with serial dilutions of test compound (0.25% v/v DMSO final) for 30 min at 37° C. Cells are stimulated with the addition of IFNγ (10 ng/ml) and lipopolysaccharide (LPS) (5 μg/ml) per well and incubated for 3 h at 37° C. Following a brief centrifugation (250×g for 2 min), supematant (10 μl) samples are taken from each well and measured against a TNFα calibration curve using a HTRF TNFα kit (CisBio) as described within. Individual IC₅₀ values of compounds are determined by nonlinear regression after fitting of curves to the experimental data using Excel XL fit 4.0 (Microsoft).

Exemplified Agents of the Invention typically suppress TNF release in this assay with an IC₅₀ of from about 1000 nM to about 10 nM or less when tested in this assay.

Agents of the invention are useful for the prevention and/or treatment of diseases, conditions and disorders that are mediated by TNF alpha and/or by MK2, including autoimmune diseases, inflammation and arthritis. The agents of the invention may also be used for example for the treatment of pain, headaches, or as an antipyretic for the treatment of fever.

In preferred uses, the agents of the invention may be used for the treatment of any of one or more of the following disorders: connective tissue and joint disorders, neoplasia disorders, cardiovascular disorders, ophthalmic disorders, respiratory disorders, gastrointestinal disorders, angiogenesis-related disorders, autoimmune and immunological disorders, allergic disorders, infectious diseases and disorders, endocrine disorders, metabolic disorders, neurological and neurodegenerative disorders, pain, hepatic and biliary disorders, musculoskeletal disorders, genitourinary disorders, gynaecological and obstetric disorders, injury and trauma disorders, muscle disorders, surgical disorders, dental and oral disorders, sexual dysfunction orders, dermatological disorders, hematological disorders, and poisoning disorders.

In other preferred embodiments, agents of the invention may be used for the prevention and treatment of autoimmune and inflammatory disorders such as arthritis (e.g. rheumatoid arthritis, psoriatic arthritis, juvenile chronic arthritis, reactive arthritis, arthritis deformans, gouty arthritis, osteoarthritis, Lyme disease), acute synovitis, autoimmune haematological disorders (e.g. hemolytic anaemia, aplastic anaemia, pure red cell anaemia and idiopathic thrombocytopenia), enterogenic spondyloarthropathies, ankylosing spondylitis, inflammatory bowel disease, ulcerative colitis, gastritis, pancreatitis, Crohn's disease, multiple sclerosis, lumbar spondylarthrosis, carpal tunnel syndrome, canine hip dysplasia, systemic lupus erythematosus, lupus nephritis, glomerulonephritis, polychondritis, scleroderma, Wegener granulamatosis, Steven-Johnson syndrome, giant cell arteritis, mixed connective tissue disease (Sharp syndrome), Reiter syndrome, rheumatic fever, dermatomyositis, polymyositis, gout, tendonitis and bursitis, organ or transplant rejection (e.g for the treatment of recipients of heart, lung, combined heart lung, liver, kidney, pancreatic, skin or corneal transplants), graft-versus-host disease, bacterial induced inflammation, sepsis, septic shock, Behcet's disease, uveitis (anterior and posterior), Muckle-Wells syndrome, psoriasis, cutaneous lupus erythematosus, dermatitis, atopic dermatitis, contact dermatitis, acne vulgaris, eczema, xerosis, type I diabetes, Graves disease, Hashimoto thyroiditis, Sjogrens syndrome, blistering disorders (e.g. pemphigus vulgaris).

In other preferred embodiments be agents of the invention may be used for the prevention and treatment of neoplasia disorders such as acral lentiginous melanoma, actinic keratoses, adenocarcinoma, adenomas, familial adenomatous polyposis, familial polyps, colon polyps, polyps, adenosarcoma, adenosquamous carcinoma, adrenocortical carcinoma, AIDS-related lymphoma, anal cancer, astrocytic tumours, batholin gland carcinoma, basal cell carcinoma, bile duct cancer, bladder cancer, brainstem glioma, brain tumours, breast cancer, bronchial gland carcinomas, capillary carcinoma, carcinoids, carcinoma, carcinomasarcoma, cavernous, central nervous system lymphoma, cerebral astrocytoma, cholangiocarcinoma, chondrosarcoma, choroid plexus papilloma/carcinoma, clear cell carcinoma, skin cancer, brain cancer, colon cancer, colorectal cancer, cutaneous T-cell lymphoma, cystadenoma, endodermal sinus tumour, endometrial hyperplasia, endometrial stromal sarcoma, endometrioid adenocarcinoma, ependymal, epitheloid, esophagal cancer, Ewing's sarcoma, extragonal germ cell tumour, fibrolamellar, focal nodular hyperplasia, gallbladder cancer, gastrinoma, germ cell tumours, gestation trophoblastic tumour, glioblastoma, hemangioblastomas, hemangiomas, hepatic adenomas, hepatic adenomatosis, hepatocellular carcinoma, Hodgkin's lymphoma, hypopharyngeal cancer, hypothalamic and visual pathway glioma, insulinoma, intraepithelial neoplasia, interepithelial cell carcinoma, Kaposi's sarcoma, kidney cancer, laryngeal cancer, leiomyosarcoma, lentigo maligna melanomas, leukaemia-related disorders, lip and oral cavity cancer liver cancer, lung cancer, lymphoma, malignant mesothelial tumours, malignant thymoma, medulloblastoma, medulloepithelioma, melanoma, meningeal, merkel cell carcinoma, mesothelial, metastatic carcinoma, mucoepidermoid carcinoma, multiple myeloma/plasma cell neoplasm, mycosis fungoides, myelodysplastic syndrome, myeloproliferative disorders, nasal cavity and paranasal sinus cancer, nasopharyngeal cancer, neuroblastoma, and neuroepithelial adenocarcinoma nodular melanoma, non-Hodgkin's lymphoma, oat cell carcinoma, oligodendroglial, oral cancer, oropharyngeal cancer, osteosarcoma, pancreatic polypeptide, ovarian cancer, ovarian germ cell cancer, pancreatic cancer, papillary serous adenocarcinoma, pineal cell, pituitary tumours, plasmacytoma, pseudosarcoma, pulmonary blastoma, parathyroid cancer, penile cancer, pheochromcytoma, dermal tumours, pituitary tumour, plasma cell neoplasm, pleuropulmonay blastoma, prostate cancer, rectal cancer, renal cell carcinoma, retinoblastoma, rhabdomysarcoma, sarcoma, serous carcinoma, small cell carcinoma, small intestine cancer, soft tissue carcinomas, somatostatin secreting tumour, squamous carcinoma, squamous cell carcinoma, submesothelial, superficial spreading carcinoma, supratentorial primitive neurectodermal tumours, thyroid cancer, undifferentiated carcinoma, urethral cancer, uterine sarcoma, uveal melanoma, verrucous carcinoma, vaginal cancer, vipoma, vulvar cancer, Waldonstrom's macroglobulinemia, well differentiated carcinoma, and Wilm's tumour.

Agents of the invention may further be used to treat or prevent cardiovascular disorders, for example myocardial ischaemia, hypertension, hypotension, heart arrhythmias, pulmonary hypertension, hypokalaemia, cardiac ischaemia, myocardial infarction, cardiac remodelling, cardiac fibrosis, myocardial necrosis, aneurysm, arterial fibrosis, embolism, vascular plaque inflammation, vascular plaque rupture, bacterial induced inflammation and viral induced inflammation, oedema, swelling, fluid accumulation, cirrhosis of the liver, Bartter's syndrome, myocarditis, arteriosclerosis, at atherosclerosis, calcification (such as vascular calcification and valvar calcification), coronary artery disease, acute coronary syndrome, heart failure, congestive heart failure, shock, arrhythmia, left ventricular hypertrophy, angina, diabetic nephropathy, kidney failure, eye damage, vascular diseases, migraine headaches, aplastic anaemia, cardiac damage, diabetic cardiac myopathy, renal insufficiency, renal injury, renal arteriography, peripheral vascular disease, left ventricular hypertrophy, cognitive dysfunction, stroke and headache.

In other preferred embodiments, agents of the invention may be used for the prevention and treatment of bone and muscle disorders such as sarcopenia, muscular dystrophy, cachexia or wasting syndrome associated with morbid TNF release (e.g. consequent to infection, cancer or organ dysfunction, especially AIDS-related cachexia), and osteoporosis.

In further preferred embodiments agents of the invention may be used for the prevention and treatment of respiratory disorders such as asthma and bronchitis, chronic obstructive pulmonary disease (COPD), cystic fibrosis, pulmonary oedema, pulmonary embolism, pneumonia, pulmonary sarcoisis, silicosis, pulmonary fibrosis, respiratory failure, acute respiratory distress syndrome, primary pulmonary hypertension and emphysema.

In further preferred embodiments, agents of the invention may be used for the prevention and treatment of the angiogenesis-related disorders selected from: angiofibroma, neovascular glaucoma, arteriovenous malformations, arthritis, Osler-Weber syndrome, atherosclerotic plaques, psoriasis, corneal graft neovascularisation, pyogenic granuloma, delayed wound healing, retrolental fibroplasias, diabetic retinopathy, stroke, cancer, AIDS complications, ulcers and infertility.

In further preferred embodiments, agents of the invention may be used for the prevention or treatment of infectious diseases and disorders such as viral infections, bacterial infections, prion infections, spiroketes infections, mycobacterial infections, rickettsial infections, chlamydial infections, parasitic infections and fungal infections.

In yet other preferred embodiments, agents of the invention may be used to the prevention and treatment of neurological and neurodegenerative disorders such as headaches, migraine, pain, dental pain, neuropathic and inflammatory pain, Alzheimer's disease, Parkinson's disease, dementia, memory loss, senility, amyotrophy, ALS, amnesia, seizures, multiple sclerosis, muscular dystrophy use, epilepsy, schizophrenia, depression, anxiety, attention deficit disorder, hyperactivity, spongiform encephalopathy, Creutzfeld-Jacob disease, Huntington's Chorea, ischaemia.

For all the above uses, an indicated daily dosage is in the range from about 0.03 to about 300 mg preferably 0.03 to 30, more preferably 0.1 to 10 mg of a compound of the invention. Agents of the Invention may be administered twice a day or up to twice a week.

The Agents of the Invention may be administered in free form or in pharmaceutically acceptable salt form. Such salts may be prepared in conventional manner and exhibit the same order of activity as the free compounds. The present invention also provides a pharmaceutical composition comprising an Agent of the Invention in free base form or in pharmaceutically acceptable salt form in association with a pharmaceutically acceptable diluent or carrier. Such compositions may be formulated in conventional manner. The Agents of the Invention may be administered by any conventional route, for example parenterally e.g. in form of injectable solutions, microemulsions or suspensions, enterally, e.g. orally, for example in the form of tablets, capsules or drinking solutions; sub-lingual, topically or transdermally, e.g. in form of a dermal cream or gel or for the purpose of administration to the eye in the form of an ocular cream, gel or eye-drop preparation, or it may be administered by inhalation.

The compounds of the invention may also be administered simultaneously, separately or sequentially in combination with one or more other suitable active agents selected from the following classes of agents: Anti IL-1 agents, e.g: Anakinra; anti cytokine and anti-cytokine receptor agents, e.g. anti IL-6 R Ab, anti IL-15 Ab, anti IL-17 Ab, anti 1L-12 Ab; B-cell and T-cell modulating drugs, e.g. anti CD20 Ab; CTL4-Ig, disease-modifying anti-rheumatic agents (DMARDs), e.g. methotrexate, leflunamide, sulfasalazine; gold salts, penicillamine, hydroxychloroquine and chloroquine, azathioprine, glucocorticoids and non-steroidal anti-inflammatories (NSAIDs), e.g. cyclooxygenase inhibitors, selective COX-2 inhibitors, agents which modulate migration of immune cells, e.g. chemokine receptor antagonists, modulators of adhesion molecules, e.g. inhibitors of LFA-1, VLA-4.

1. A compound of formula (I) or a pharmaceutically acceptable salt thereof: 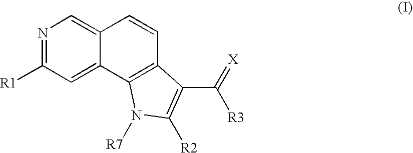
wherein
R1 is halo, cyano, hydroxyl, mercapto, or aryl, aryl-C₁-C₆ alkyl, aryl-C₂-C₆ alkenyl, monocyclic heteroaryl, heteroaryl-C₁-C₆ alkyl, heteroaryl-C₂-C₆ alkenyl, arylamino, heteroarylamino, aryloxy, heteroaryloxy, C₁-C₆ alkyl, C₃-C₇ cycloalkyl, C₁-C₆ alkoxy, C₁-C₆ alkylamino, C₂-C₆ alkenyl, C₂-C₆ alkynyl, heterocycloalkyl, heterocycloalkyl-C₁-C₆ alkyl, heterocycloalkyl-C₂-C₆ alkenyl, heterocycloalkylamino, heterocycloalkyloxy, amino, which are optionally substituted with substituents independently selected from the group consisting of:
halo, cyano, hydroxyl, mercapto, sulfonyl, amino, C₁-C₆ alkylamino, di-C₁-C₆ alkylamino, aryl, monocyclic heteroaryl, C₁-C₆ alkyl, C₁-C₆ alkoxy, C₃-C₇ cycloalkyl, C₃-C₇ heterocycloalkyl, carboxyl, carbonyl C₁-C₇ alkyl, each of which, where applicable, may be optionally substituted by C₁-C₆ alkyl, C₃-C₇ cycloalkyl, C₃-C₇ cycloalkyl, C₃-C₇ heterocycloalkyl, C₁-C₆ alkoxy, C₁-C₆ alkenyl, C₁-C₆ alkynyl, halo, hydroxyl, mercapto, cyano, amino, C3-C7 heterocycloalkyl carbonyl; each of which, where applicable, may be optionally substituted by C₁-C₆ alkyl, C₃-C₇ cycloalkyl, C₃-C₇ cycloalkyl, C₃-C₇ heterocycloalkyl, C₁-C₆ alkoxy, C₁-C₆ alkenyl, C₁-C₆ alkynyl, halo, hydroxyl, mercapto, cyano, amino and C3-C7 heterocycloalkyl carbonyl;
X is O, S or NOH;
R2 represents the group —C(A)(Q)-Y
wherein Q is H or C₁-C₆ alkyl;
A is H or C₁-C₆ alkyl;
Y is amino, aminooxy, hydroxyl, C₁-C₆ alkoxy, C₁-C₆ alkylamino or hydrazine which in each case may be optionally substituted with C₁-C₆ alkyl, halo or hydroxyl;
R3 is —OH, —OR4 or —NHR4,
wherein R4 is H or C₁-C₆ alkyl;
or R2 and R3 are joined to denote collectively the group —R2-R3- to form a 5-, 6 or 7-membered ring, the collective group —R2-R3- is
—(CH₂)ₙNR5-, —CH₂ONH—, —(CH₂)ₙ—, —CH═N—NH—, or —(CH₂)ₙ—(CH₂)ₙ—NR6-NH—
wherein R5 is H, C₁-C₆ alkyl, aryl-C₁-C₆ alkyl, heteroaryl-C₁-C₆ alkyl, C₃-C₇ cylcloakyl-C₁-C₆ alkyl, or C₃-C₇ heterocylcloalkyl-C₁-C₆ alkyl, which are optionally substituted with one or more groups independently selected from the group consisting of halo, C₁-C₆ alkyl, C₁-C₆ alkoxy, amino, trifluoromethyl, sulfonyl and hydroxyl;
and R6 is H or C₁-C₆ alkyl, carbonyl, sulfonyl; which are optionally substituted with one or more groups independently selected from the group consisting of C₁-C₆ alkyl, lower alkoxy, amino, alkylamino and hydroxyl;
wherein n is 1, 2 or 3;
and R7 is H or C₁-C₆ alkyl, which is optionally substituted with amino, hydroxyl, halo or carboxy.

2. The compound according to claim 1 wherein R1 is halo or aryl, monocyclic heteroaryl, aryl-C₂-C₆ alkenyl, aryloxy, C₁-C₆ alkylamino), which in each case may be optionally substituted.

3. The compound according to claim 1 wherein R1 is halo or; phenyl, pyridyl or styryl which in each case may be optionally substituted.

4. The compound according to claim 1 wherein X is O.

5. The compound according to claim 1 wherein R2 represents the group —C(A)(Q)-Y wherein A and Q are H or C₁-C₆ alkyl; and Y is amino, aminooxy, C₁-C₆ alkylamino or hydrazine which in each case may be optionally substitute with C₁-C₆ alkyl, halo or hydroxyl.

6. The compound according to claim 1 wherein R2 and R3 are joined to denote collectively the group —R2-R3- to form a 5-, 6 or 7-membered ring, wherein —R2-R3- is;
—(CH₂)ₙNR5-, —CH₂ONH—, —(CH₂)ₙ—, —CH═N—NH—or —(CH₂)ₙ—NH—NH—
wherein R5 is H or C₁-C₆ alkyl, aryl-C₁-C₆ alkyl, heteroaryl-C₁-C₆ alkyl, C₃-C₇ cylcloakyl-C₁-C₆ alkyl, C₃-C₇heterocylcloalkyl-C₁-C₆ alkyl, each of which are optionally substituted with one or more groups independently selected from the group consisting of halo, C₁-C₆ alkyl, C₁-C₆ alkoxy, amino, trifluoromethyl, sulfonyl and hydroxyl,
and wherein n is 1, 2 or 3.

7. The compound according to claim 1, wherein the compound is:
2-Aminomethyl-8-((E)-styryl)-1H-pyrrolo[2,3-f]isoquinoline-3-carboxylic acid hydrochloride,
2-((E)-Styryl)-9,10-dihydro-8H-3,8,10-triaza-pentaleno[2,1-a]naphthalen-7-one,
2-Aminomethyl-8-chloro-1H-pyrrolo[2,3-f]isoquinoline-3-carboxylic acid hydrochloride,
2-Chloro-9,10-dihydro-8H-3,8,10-triaza-pentaleno[2,1-a]naphthalen-7-one,
2-Aminooxymethyl-8-((E)-styryl)-1H-pyrrolo[2,3-f]isoquinoline-3-carboxylic acid hydrochloride,
2-((E)-Styryl)-10,11-dihydro-9-oxa-3,8,11-triaza-benzo[a]fluoren-7-one,
2-Chloro-8,9,10,11-tetrahydro-pyrido[4,3-a]carbazol-7-one,
2-Chloro-8,9,10,11-tetrahydro-pyrido[4,3-a]carbazol-7-one oxime,
2-Chloro-9,10,11,12-tetrahydro-8H-3,8,12-triaza-naphtho[2,1-a]azulen-7-one,
2-((E)-Styryl)-9,10,11,12-tetrahydro-8H-3,8,12-triaza-naphtho[2,1-a]azulen-7-one,
2-(4-Fluoro-phenyl)-9,10,11,12-tetrahydro-8H-3,8,12-triaza-naphtho[2,1-a]azulen-7-one,
2-Aminooxymethyl-8-chloro-1H-pyrrolo[2,3-f]isoquinoline-3-carboxylic acid hydrochloride,
2-Chloro-10,11-dihydro-9-oxa-3,8,11-triaza-benzo[a]fluoren-7-one,
8-Chloro-2-hydrazinomethyl-1H-pyrrolo[2,3-f]isoquinoline-3-carboxylic acid hydrochloride,
2-Chloro-8,11-dihydro-3,8,9,11-tetraaza-benzo[a]fluoren-7-one,
2-Chloro-8,9,10,11-tetrahydro-3,8,9,11-tetraaza-benzo[a]fluoren-7-one,
2-(4-Methoxy-phenyl)-9-methyl-8,9,10,11-tetrahydro-3,8,9,11-tetraaza-benzo[a]fluoren-7-one,
2-(4-Methoxyphenyl)-9,10,11,12-tetrahydro-8H-3,8,12-triaza-naphtho[2,1-a]azulen-7-one,
2-Methoxymethyl-8-((E)-styryl)-1H-pyrrolo[2,3-f]isoquinoline-3-carboxylic acid amide, 2-Methylaminomethyl-8-((E)-styryl)-1H-pyrrolo[2,3-f]isoquinoline-3-carboxylic acid hydrochloride,
8-Methyl-2-((E)-styryl)-9,10-dihydro-8H-3,8,10-triaza-pentaleno[2,1-a]naphthalen-7-one,
2-(4-Hydroxy-phenyl)-9,10-dihydro-8H-3,8,10-triaza-pentaleno[2,1-a]naphthalen-7-one,
2-[(E)-2-(4-Methoxy-phenyl)-vinyl]-9,10-dihydro-8H-3,8,10-triaza-pentaleno[2,1-a]naphthalen-7-one,
2-[(E)-2-(4-Morpholin-4-ylmethyl-phenyl)-vinyl]-9,10-dihydro-8H-3,8,10-triaza-pentaleno[2,1-a]naphthalen-7-one,
2-{(E)-2-[3-(2-Morpholin-4-yl-ethyl)-phenyl]-vinyl}-9,10-dihydro-8H-3,8,10-triaza-pentaleno[2,1-a]naphthalen-7-one,
8-Benzyl-2-((E)-styryl)-9,10-dihydro-8H-3,8,10-triaza-pentaleno[2,1-a]naphthalen-7-one,
2-(3-Methoxy-phenyl)-9,10-dihydro-8H-3,8,10-triaza-pentaleno[2,1-a]naphthalen-7-one,
2-[3-(3-Methoxy-propoxy)-phenyl]-9,10-dihydro-8H-3,8,10-triaza-pentaleno[2,1-a]naphthalen-7-one,
2-Pyridin-3-yl-9,10-dihydro-8H-3,8,10-triaza-pentaleno[2,1-a]naphthalen-7-one,
2-(4-Methoxy-phenyl)-9,10-dihydro-8H-3,8,10-triaza-pentaleno[2,1-a]naphthalen-7-one,
2-(2-Fluoro-phenyl)-9,10-dihydro-8H-3,8,10-triaza-pentaleno[2,1-a]naphthalen-7-one,
2-(3-Methanesulfonyl-phenyl)-9,10-dihydro-8H-3,8,10-triaza-pentaleno[2,1-a]naphthalen-7-one,
2-(2-Trifluoromethyl-phenyl)-9,10-dihydro-8H-3,8,10-triaza-pentaleno[2,1-a]naphthalen-7-one,
2-(3-Fluoro-phenylamino)-9,10-dihydro-8H-3,8,10-triaza-pentaleno[2,1-a]naphthalen-7-one,
2-[(E)-2-(4-Morpholin-4-ylmethyl-phenyl)-vinyl]-9,10,11,12-tetrahydro-8H-3,8,12-triaza-naphtho[2,1-a]azulen-7-one,
2-Pyridin-3-yl-9,10,11,12-tetrahydro-8H-3,8,12-triaza-naphtho[2,1-a]azulen-7-one,
2-Chloro-8,9,10,11-tetrahydro-3,8,11-triaza-benzo[a]fluoren-7-one,
2-{(E)-2-[4-(2-Hydroxy-2-methyl-propoxy)-phenyl]-vinyl}-8,9,10,11-tetrahydro-3,8,11-triaza-benzo[a]fluoren-7-one,
2-[(E)-2-(3-Morpholin-4-yl-phenyl)-vinyl]-8,9,10,11-tetrahydro-3,8,11-triaza-benzo[a]fluoren-7-one,
2-[(E)-2-(3-Morpholin-4-yl-phenyl)-vinyl]-9,10-dihydro-8H-3,8,10-triaza-pentaleno[2,1-a]naphthalen-7-one,
2-[(E)-2-(3-Morpholin-4-yl-phenyl)-vinyl]-9,10,11,12-tetrahydro-8H-3,8,12-triaza-naphtho[2,1-a]azulen-7-one,
2-[(E)-2-(3-Morpholin-4-ylmethyl-phenyl)-vinyl]-9,10-dihydro-8H-3,8,10-triaza-pentaleno[2,1-a]naphthalen-7-one,
2-[(E)-2-(3-Morpholin-4-ylmethyl-phenyl)-vinyl]-8,9,10,11-tetrahydro-3,8,11-triaza-benzo[a]fluoren-7-one,
2-[(E)-2-(3-Morpholin-4-ylmethyl-phenyl)-vinyl]-9,10,11,12-tetrahydro-8H-3,8,12-triaza-naphtho[2,1-a]azulen-7-one,
2-{(E)-2-[3-(2-Morpholin-4-yl-ethyl)-phenyl]-vinyl}-8,9,10,11-tetrahydro-3,8,11-triaza-benzo[a]fluoren-7-one,
2-{(E)-2-[3-(2-Morpholin-4-yl-ethyl)-phenyl]-vinyl}-9,10,11,12-tetrahydro-8H-3,8,12-triaza-naphtho[2,1-a]azulen-7-one,
2-{(E)-2-[3-(2-Morpholin-4-yl-2-oxo-ethyl)-phenyl]vinyl}-9,10-dihydro-8H-3,8,10-triaza-pentaleno[2,1-a]naphthalen-7-one,
2-{(E)-2-[3-(2-Morpholin-4-yl-2-oxo-ethyl)-phenyl]-vinyl}-8,9,10,11-tetrahydro-3,8,11-triaza-benzo[a]fluoren-7-one,
2-{(E)-2-[3-(2-Morpholin-4-yl-2-oxo-ethyl)-phenyl]-vinyl}-9,10,11,12-tetrahydro-8H-3,8,12-triaza-naphtho[2,1-a]azulen-7-one,
2-((E)-2-{3-[2-(4-Methyl-piperazin-1-yl)-2-oxo-ethyl]-phenyl}-vinyl)-9,10-dihydro-8H-3,8,10-triaza-pentaleno[2,1-a]naphthalen-7-one,
2-((E)-2-{3-[2-(4-Methyl-piperazin-1-yl)-2-oxo-ethyl]-phenyl}-vinyl)-8,9,10,11-tetrahydro-3,8,11-triaza-benzo[a]fluoren-7-one,
2-((E)-2-{3-[2-(4-Methyl-piperazin-1-yl)-2-oxo-ethyl]-phenyl}-vinyl)-9,10,11,12-tetrahydro-8H-3,8,12-triaza-naphtho[2,1-a]azulen-7-one,
11-Methyl-2-[(E)-2-(3-morpholin-4-yl-phenyl)-vinyl]-8,9,10,11-tetrahydro-3,8,11-triaza-benzo[a]fluoren-7-one,
11-Methyl-2-{(E)-2-[3-(2-morpholin-4-yl-ethyl)-phenyl]-vinyl}-8,9,10,11-tetrahydro-3,8,11-triaza-benzo[a]fluoren-7-one,
1,1-Dimethyl-4-(2-{3-[(E)-2-(10-methyl-7-oxo-7,8,9,10-tetrahydro-3,8,10-triaza-pentaleno[2,1-a]naphthalen-2-yl)-vinyl]-phenyl}-acetyl)-piperazin-1-ium iodide,
2-[(E)-2-(4-Morpholin-4-ylmethyl-phenyl)-vinyl]-8,9,10,11-tetrahydro-3,8,11-triaza-benzo[a]fluoren-7-one,
2-[3-(2-Morpholin-4-yl-ethoxy)-phenyl]-9,10-dihydro-8H-3,8,10-triaza-pentaleno[2,1-a]naphthalen-7-one,
2-[3-(2-Morpholin-4-yl-ethoxy)-phenyl]-8,9,10,11-tetrahydro-3,8,11-triaza-benzo[a]fluoren-7-one,
2-[3-(2-Morpholin-4-yl-ethoxy)-phenyl]-9,10,11,12-tetrahydro-8H-3,8,12-triaza-naphtho[2,1-a]azulen-7-one,
2-{4-[2-(4-Methyl-piperazin-1-yl)-2-oxo-ethyl]-phenyl}-8,9,10,11-tetrahydro-3,8,11-triaza-benzo[a]fluoren-7-one,
2-{3-[2-(4-Methyl-piperazin-1-yl)-2-oxo-ethyl]-phenyl}-8,9,10,11-tetrahydro-3,8,11-triaza-benzo[a]fluoren-7-one,
2-{3-[2-(4-Methyl-piperazin-1-yl)-2-oxo-ethyl]-phenyl}-9,10,11,12-tetrahydro-8H-3,8,12-triaza-naphtho[2,1-a]azulen-7-one,
2-[5-(2-Morpholin-4-yl-ethoxy)-pyridin-3-yl]-9,10-dihydro-8H-3,8,10-triaza-pentaleno[2,1-a]naphthalen-7-one,
2-[5-(2-Morpholin-4-yl-ethoxy)-pyridin-3-yl]-8,9,10,11-tetrahydro-3,8,11-triaza-benzo[a]fluoren-7-one,
2-[5-(2-Morpholin-4-yl-ethoxy)-pyridin-3-yl]-9,10,11,12-tetrahydro-8H-3,8,12-triaza-naphtho[2,1-a]azulen-7-one,
2-[5-(2-Methoxy-ethoxy)-pyridin-3-yl]-9,10,11,12-tetrahydro-8H-3,8,12-triaza-naphtho[2,1-a]azulen-7-one,
2-[5-(2-Methoxy-ethoxy)-pyridin-3-yl]-9,10-dihydro-8H-3,8,10-triaza-pentaleno[2,1-a]naphthalen-7-one,
2-[5-(2-Methoxy-ethoxy)-pyridin-3-yl]-8,9,10,11-tetrahydro-3,8,11-triaza-benzo[a]fluoren-7-one,
2-Pyridin-3-yl-9,10,11,12-tetrahydro-8H-3,8,12-triaza-naphtho[2,1-a]azulen-7-one,
2-(5-Methoxy-pyridin-3-yl)-9,10-dihydro-8H-3,8,10-triaza-pentaleno[2,1-a]naphthalen-7-one,
2-(6-Methoxy-pyridin-3-yl)-9,10-dihydro-8H-3,8,10-triaza-pentaleno[2,1-a]naphthalen-7-one,
2-(6-Dimethylamino-pyridin-3-yl)-9,10-dihydro-8H-3,8,10-triaza-pentaleno[2,1-a]naphthalen-7-one,
2-{(E)-2-[4-(2-Hydroxy-2-methyl-propoxy)-phenyl]-vinyl}-9,10,11,12-tetrahydro-8H-3,8,12-triaza-naphtho[2,1-a]azulen-7-one,
2-(3-Fluoro-4-methoxy-phenyl)-8,9,10,11-tetrahydro-3,8,11-triaza-benzo[a]fluoren-7-one,
2-(3-Chloro-4-propoxy-phenyl)-8,9,10,11-tetrahydro-3,8,11-triaza-benzo[a]fluoren-7-one,
2-(3-Fluoro-4-methoxy-phenyl)-9,10-dihydro-8H-cyclopenta[4,5]pyrrolo[2,3-f]isoquinolin-7-one,
2-(3-Chloro-4-propoxy-phenyl)-9,10-dihydro-8H-cyclopenta[4,5]pyrrolo[2,3-f]isoquinolin-7-one,

8-10. (canceled)

11. A method of treatment of cytokine mediated conditions comprising:
administering to a patient in need thereof, an effective amount of a the compound of formula (I) according to claim 1, or a pharmaceutically-acceptable salt thereof.

12. A pharmaceutical composition, comprising:
a the compound of formula (I) according to claim 1, or a pharmaceutically-acceptable salt thereof, and
a pharmaceutically acceptable excipient, diluent or carrier.

13. (canceled)

14. A combination, comprising:
a the compound according to claim 1 in combination with one or more active agents selected from the following: Anti IL-1 agents, anti cytokine and anti-cytokine, receptor agents, B-cell and T-cell modulating drugs, disease-modifying anti-rheumatic agents (DMARDs), gold salts, penicillamine, hydroxychloroquine and chloroquine, azathioprine, glucocorticoids, non-steroidal anti-inflammatories (NSAIDs), selective COX-2 inhibitors, agents which modulate migration of immune cells, chemokine receptor antagonists, modulators of adhesion molecules, for simultaneous, separate or sequential administration.

15. The method of claim 11, wherein said condition is an autoimmune disease, inflammation or arthritis.
